(19) United States
(12) Patent Application Publication
(10) Pub. No.: US 2026/0177547 A1
(43) Pub. Date: Jun. 25, 2026

(54) CYCLOOCTYNE-MODIFIED N-HETEROCYCLIC CARBENE COMPOUNDS USEFUL FOR BIOMOLECULE IMMOBILIZATION ON METAL SURFACES

(71) Applicants: University of Tennessee Research Foundation, Knoxville, TN (US); University of Notre Dame du Lac, South Bend, IN (US); The Johns Hopkins University, Baltimore, MD (US)

(72) Inventors: David Jenkins, Knoxville, TN (US); Phattananawee Nalaoh, Knoxville, TN (US); Jon Camden, South Bend, IN (US); Netzahualcoyotl Arroyo-Curras, Baltimore, MD (US); Vincent Clark, Baltimore, MD (US)

(73) Assignees: University of Tennessee Research Foundation, Knoxville, TN (US); University of Notre Dame du Lac, South Bend, IN (US); The Johns Hopkins University, Baltimore, MD (US)

(21) Appl. No.: 19/427,538

(22) Filed: Dec. 19, 2025

Related U.S. Application Data
(60) Provisional application No. 63/737,245, filed on Dec. 20, 2024.

(51) Int. Cl. G01N 33/543; C07D 235/06; C07D 403/12; C12N 15/115; G01N 33/53

(52) U.S. Cl. CPC G01N 33/5438; C07D 235/06; C07D 403/12; C12N 15/115; G01N 33/5308; C12N 2310/16

(57) ABSTRACT

The present disclosure relates to cyclooctyne-functionalized N-heterocyclic carbene (NHC) compounds and compositions, methods of preparing these NHC compounds and compositions, and electrochemical aptamer-based biosensors (E-ABs) that incorporate these NHC compounds and methods of their use.

### CROSS REFERENCE TO RELATED APPLICATIONS

This application claims priority benefit to U.S. Provisional Application No. 63/737,245, filed Dec. 20, 2024, which is hereby incorporated by reference herein.

### GOVERNMENT LICENSE RIGHTS

This invention was made with government support under contracts CHE2108328 and CHE2108330 awarded by National Science Foundation and contract GM140143 awarded by the National Institutes of Health. The government has certain rights in the invention.

### FIELD

The present disclosure relates to cyclooctyne-functionalized N-heterocyclic carbene (NHC) compounds and compositions, methods of preparing these NHC compounds and compositions, and electrochemical aptamer-based biosensors (E-ABs) that incorporate these NHC compounds and methods of their use.

### BACKGROUND

Continuous, real-time, and in vivo monitoring of molecular targets in healthy and diseased states is a holy grail in bioanalytical chemistry. Among the many approaches under development, those employing electrochemistry have proved to be highly successful and commercially viable platforms for real-time molecular monitoring. For example, continuous glucose sensors, use oxidase-reductase enzymes to catalytically convert glucose to electrons for electrochemical detection. Similarly, direct voltametric oxidation of a target species can provide a real-time electrochemical probe. For example, measurement of the catecholamines or hydrogen peroxide in brain tissue. These direct or biocatalysis-mediated electrochemical methods, however, are not applicable to many clinically critical biomarkers and the methods with the ability to probe the complex chemical dynamics of human physiology and disease are limited.

Electrochemical aptamer-based sensors (E-ABs) utilize reversible affinity interactions, instead of reactivity, to achieve continuous molecular monitoring in vivo. Utilizing aptamers as the sensor recognition element yields a highly modular biosensing platform, since substituting a new target-specific aptamer does not require a reengineering of the sensor architecture. Thus, E-ABs can potentially provide a platform that vastly expands the scope of continuous, real-time, in vivo electrochemical monitoring.

Current E-AB technology relies on gold-thiol bonds (Au—S) to create a mixed self-assembled monolayer (SAM) combining an electrode-blocking hydrocarbon chain and a redox-reporter-modified aptamer on a gold electrode (see e.g., FIG. 1A). The thiol monolayer consists of a hydrophobic blocking portion, capable of reducing background electrochemical processes, and a hydrophilic terminal group to reduce non-specific binding and biofouling. The aptamers, also anchored via thiols, are engineered to undergo a reversible conformational change upon target binding; thereby modulating the rate of electron transfer between a reporter (e.g., methylene blue) and the electrode. The sensor response time is determined by the dynamic equilibrium between the targets, aptamers, and target-aptamer complexes and is established in milliseconds. E-ABs are interrogated by measuring redox currents that are calibrated to a target via titration. Reversible sensing is achieved by serial interrogation of the E-ABs, for example, using square-wave voltammetry (see e.g., FIG. 1B) every 10 seconds where the oxidation state of the reporter is regenerated with each voltametric scan. The voltametric peak currents can then be converted to concentrations in real time using open-source software.

E-ABs have been used to continuously monitor systemic concentrations of over half a dozen molecular targets in live-rodent veins, illustrating the value of the approach for continuous in vivo molecular measurements (see e.g., 1-13). Redox-reporter-modified aptamers have been shown to be capable of binding with high affinity to specific analytes, including ions (14) small molecules (15,16), proteins (17,18), and even whole entities such as viruses (19). FIG. 1A depicts a schematic representation of an E-AB constructed with SAMs on an electrode and how it can detect a target molecule via electrochemical changes in the redox-reporter modified aptamer. Target binding to the aptamer modulates electron transfer (20) from the aptamer-bound redox-reporter moiety to the electrode in proportion to the concentration of the target. This process is reversible and equilibrates with time constants of milliseconds (21,22), allowing for real-time monitoring of changing target concentrations. FIG. 1B shows the typical square wave voltammagrams from the E-AB electrode showing how aptamer binding induces changes in the electron transfer kinetics to yield a local target concentration which allows for continuous molecular monitoring in vivo.

E-ABs are typically constructed via thiol self-assembled monolayers (SAMs) on gold electrodes consisting of redox-reporter-modified aptamers (such as oligonucleotides) diluted within the alkylthiol monolayer (23-26). Despite the demonstrated real-time in vivo monitoring potential of E-ABs, device lifetimes have been limited by the stability of the thiol monolayers on gold, as the rate of thiol desorption greatly exceeds the rate of enzymatic cleavage of tethered aptamers. The thiol SAMs have been observed to suffer from low stability in air (27-29) are temperature-sensitive (30-32) and degrade in biological media (33,34) due to the poor stability of the thiol-gold bond. It has been demonstrated that thiols are a poor fit for long-term E-AB operation, since they can suffer from competitive displacement in biofluids (e.g., ligand exchange with cysteine) and voltage induced desorption during sensing (35-41). Modified aptamers exist that can fully resist nuclease hydrolysis in biofluids for well over seven days, yet the susceptibility of thiol monolayers to desorption and biofouling renders the sensors inoperable within hours. Moreover, the repeated electrochemical interrogation required for continuous monitoring accelerates the loss of thiols from the electrode surface, further shortening E-AB operational life. This limited lifespan of thiol monolayers represents a major obstacle to in vivo monitoring using E-ABs.

N-heterocyclic carbene (NHC) compounds have been incorporated in a number of technologies that rely on monolayer self-assembly (42-48). NHCs form strong σ-bonds to gold, especially on gold surfaces (49-51) which provides NHCs monolayers with incredible stability as compared to alkylthiol monolayers (52,53). Previous research has demonstrated that NHCs are stable in biofluids over long periods (54,55) and stable to long-term electrochemical cycling (56,57). An electrochemical sensor for pathogen detection employing NHCs has been reported but this construction of this sensor required a multistep post-synthetic modification of an amine-tailed NHC on the gold electrode surface (58).

Accordingly, there remains a need for new compositions and methods that can be used to construct E-ABs with increased stability during exposure to biological environments under continuous voltage cycling.

### SUMMARY

The present disclosure generally relates to cyclooctyne-functionalized N-heterocyclic carbene (NHC) compounds, and biosensors, including electrochemical aptamer-based biosensors (E-ABs), that are prepared with and incorporate these compounds. NHCs are monolayer-forming ligands that offer improved stability versus thiols when attached to surfaces, such as metal electrodes, for use in biological applications. The modification of NHCs with cyclooctyne groups, such as bicyclo[6.1.0]nonyne (“BCN”) and dibenzoazacyclooctyne (“DBCO”), allow the NHCs to undergo strain-promoted azide-alkyne cycloaddition (SPAAC) reactions with a broad array of azide-modified biomolecules, such as aptamers, in the construction of stable E-AB biosensors. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides an N-heterocyclic carbene (NHC) compound of structural formula I

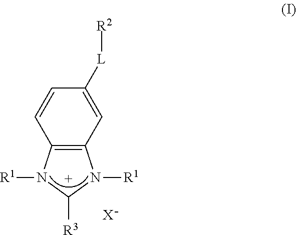

wherein, R¹ is selected from —H, —CH₃, —CH₂—(CH₃)₂, —(C₁-C₆) linear or branched alkyl; R² is a cyclooctyne group; L is a linker comprising a covalently bonded chain of 2 to 100 atoms; R³ is selected from —H and —COO⁻; and X⁻ is an optional anion present when R³ is —H.

In at least one embodiment of the NHC compounds of the present disclosure, R¹ is selected from —CH₃ and —CH₂—(CH₃)₂.

In at least one embodiment of the NHC compounds of the present disclosure, the linker L comprises one or more of the following chemical groups: linear (C₁-C₅) alkyl, linear (C₁-C₅) alkenyl, linear (C₁-C₅) alkynyl, ester, ether, amine, amide, imide, phosphodiester, and/or polyethylene glycol (PEG). In at least one embodiment, L comprises a linker of structural formula L_{A}, L_{B}, or L_{C}

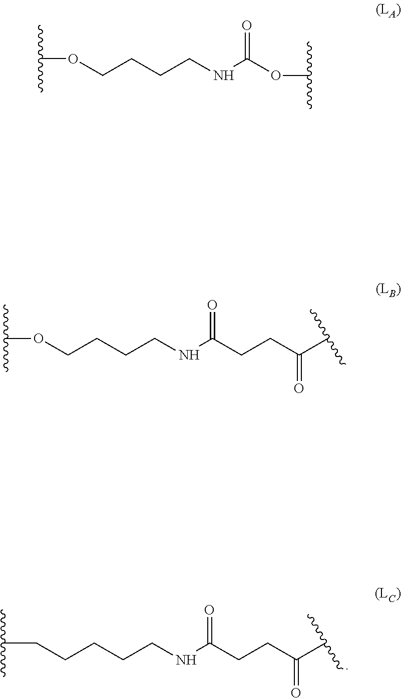 [SMILES: *OCCCCNC(=O)O* ; *OCCCCNC(=O)CCC(*)=O ; *CCCCCNC(=O)CCC(*)=O]

In at least one embodiment of the NHC compounds of the present disclosure, R² is a cyclooctyne group selected from IIa and IIb:

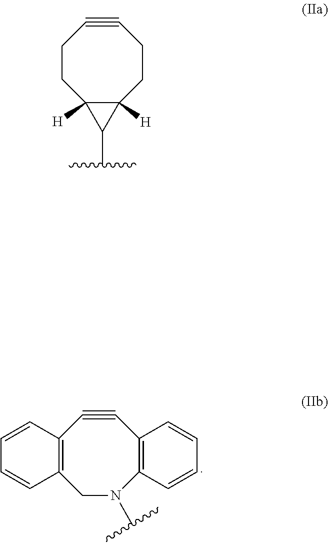 [SMILES: *C1[C@H]2CCC#CCC[C@@H]12 ; *N1Cc2ccccc2C#Cc2ccccc21]

In at least one embodiment of the NHC compounds of the present disclosure, R³ is —H and the X⁻ is an anion selected from a halide and HCO₃⁻. In at least one embodiment, R³ is —H and X⁻ is a halide selected from F⁻, Cl⁻, Br⁻, and I⁻.

In at least one embodiment of the NHC compounds of the present disclosure, R³ is —COO⁻.

In at least one embodiment of the NHC compounds of the present disclosure, the NHC compound of structural formula I is selected from compounds Ia, Ib, Ic, Id, Ie, If, Ig, and Ih:

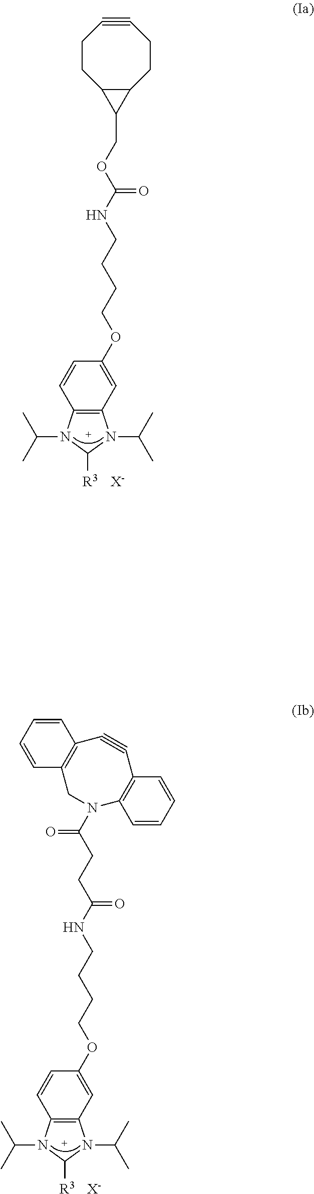

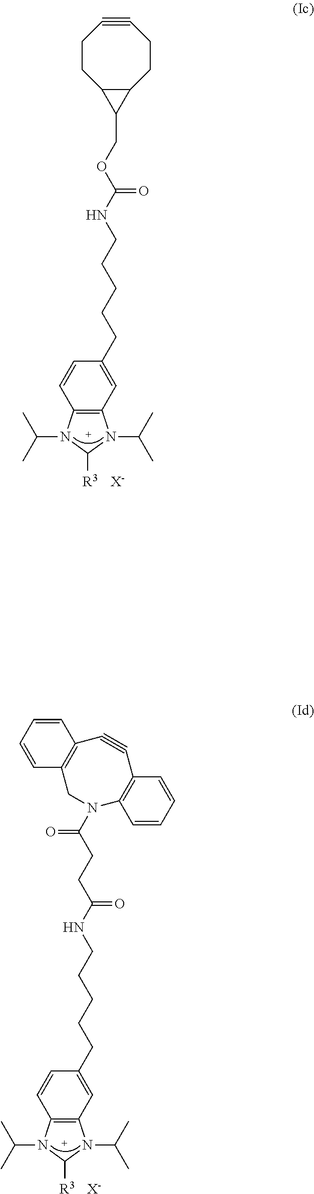

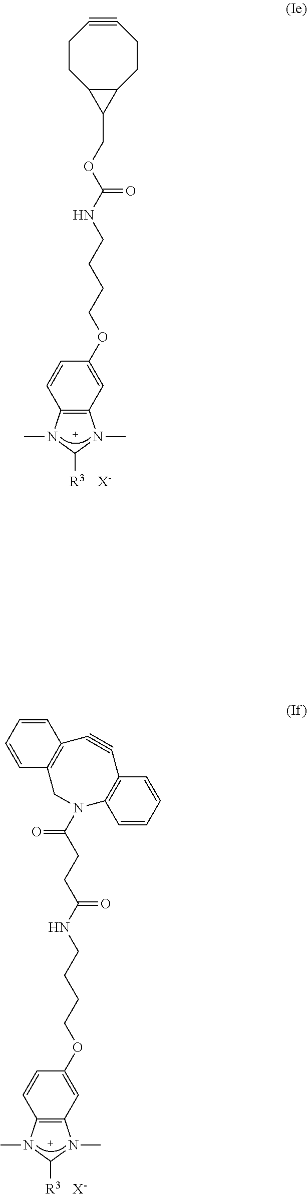

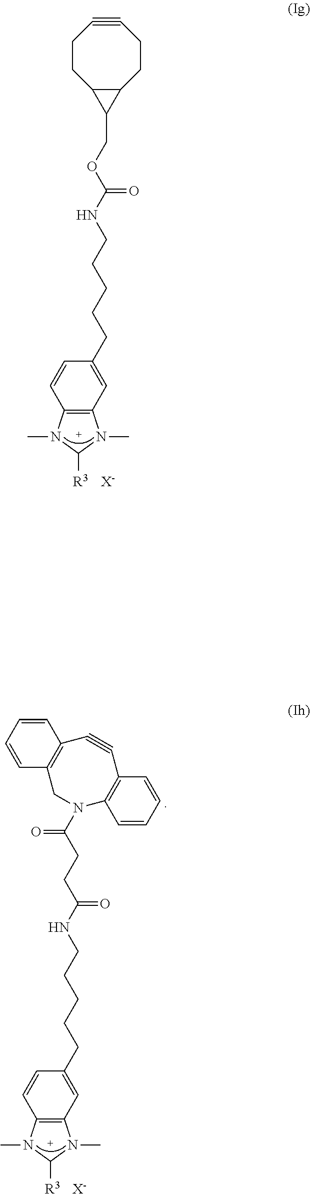

In at least one embodiment of the NHC compounds of the present disclosure, the compound is selected from compounds 5, 6, 7, 8, 9, and 10:

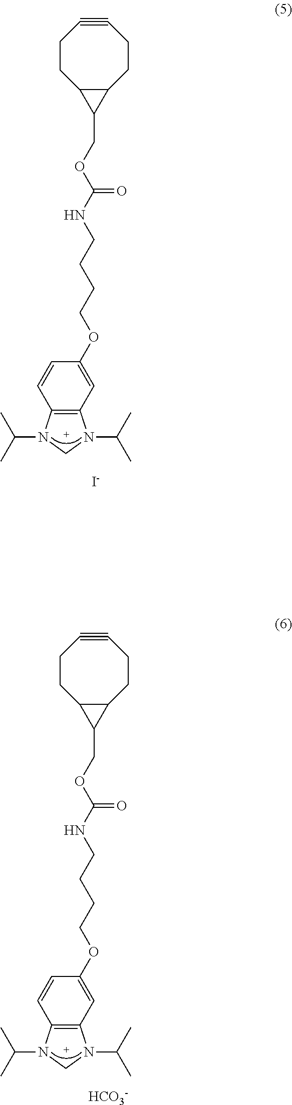

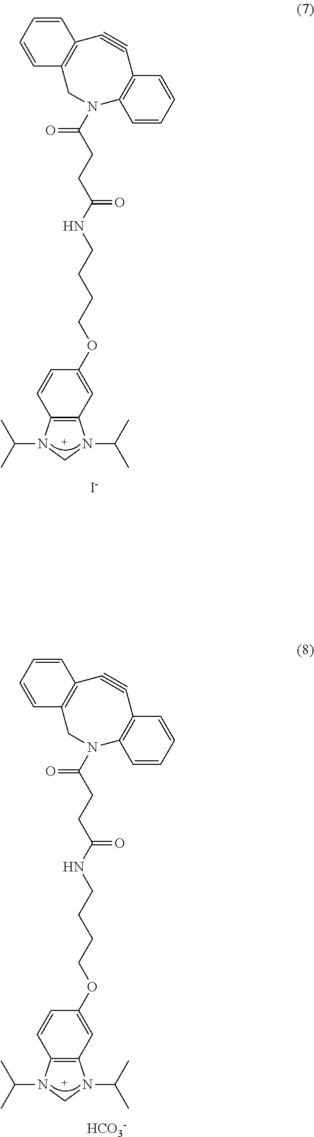

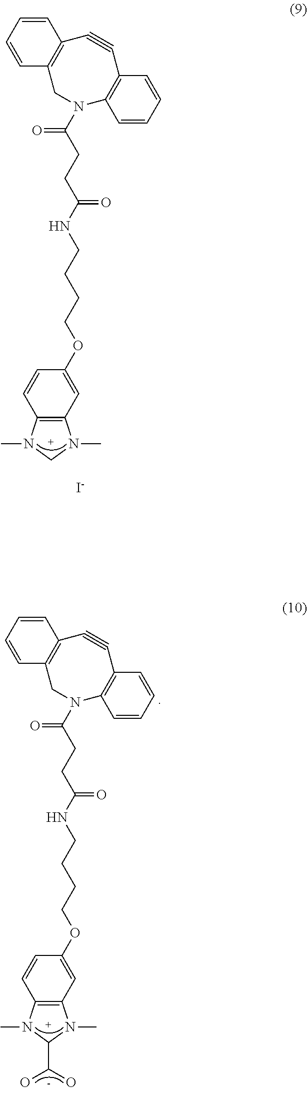

In another aspect, the present disclosure provides a method of preparing an N-heterocyclic carbene (NHC) compound of structural formula I, wherein, R¹ is selected from —CH₃, and —CH₂—(CH₃)₂; R² is a cyclooctyne group selected from IIa and IIb; L comprises a linker of structural formula L_{A} or L_{B}; R³ is selected from —H and —COO⁻; and X⁻ is an optional anion that is present when R³ is —H; the method comprising:
(a) preparing a Boc-protected precursor compound 3 or 4 according to the synthesis steps of Scheme 1:

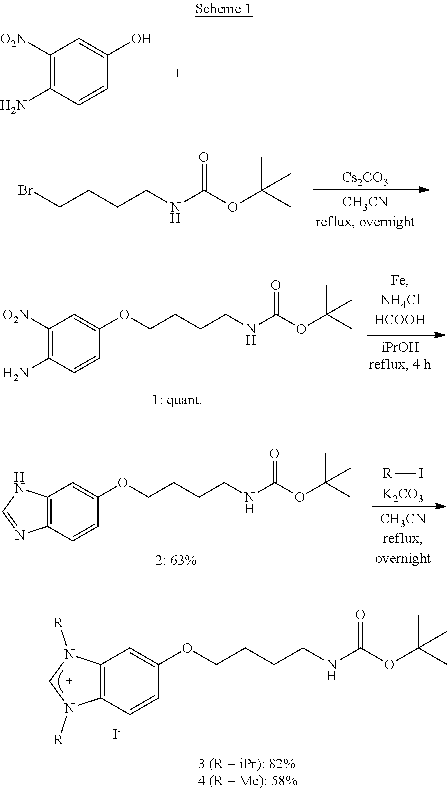

(b) reacting the precursor compound 3 of step (a) with BCN—NHS to prepare a BCN-functionalized NHC iodide salt of compound 5 or bicarbonate salt of compound 6, or with DBCO—NHS to prepare a DBCO-functionalized NHC iodide salt of compound 7 or bicarbonate salt of compound 8 according to the synthesis steps of Scheme 2A:

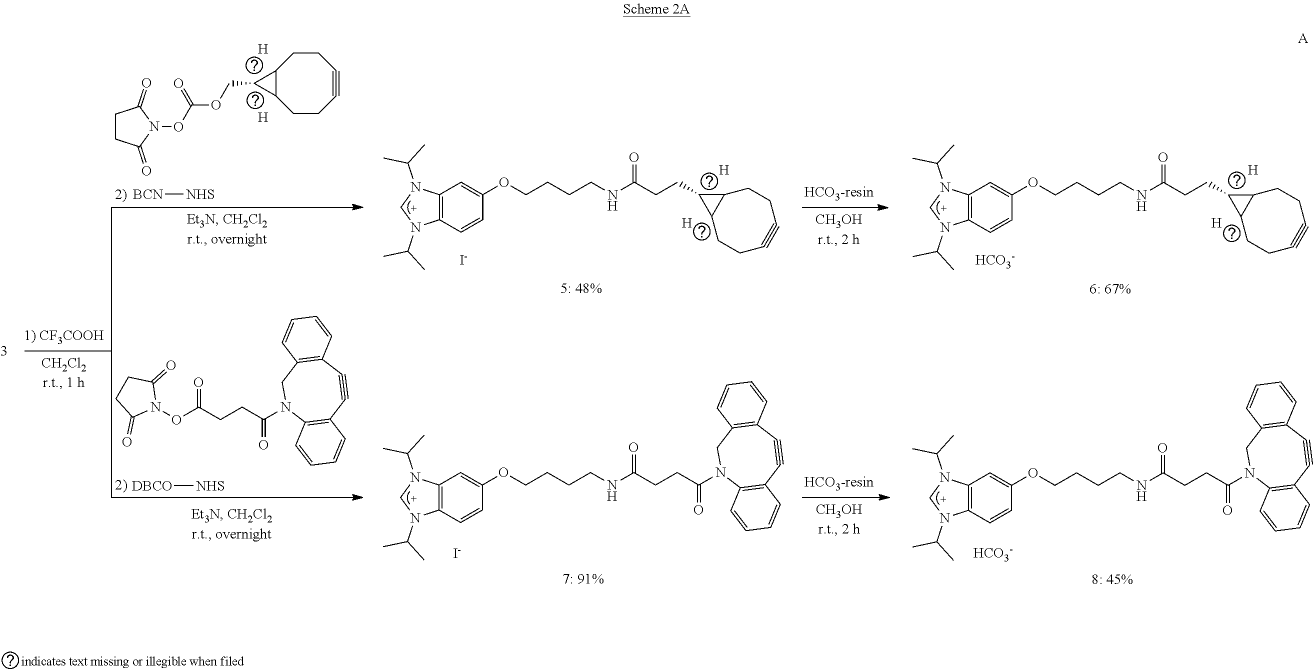

or
reacting the precursor compound 4 of step (a) with DBCO—NHS to prepare a DBCO-functionalized NHC iodide salt of compound 9 or a DBCO-functionalized NHC carboxylate adduct of compound 10, according to the synthesis steps of Scheme 2B:

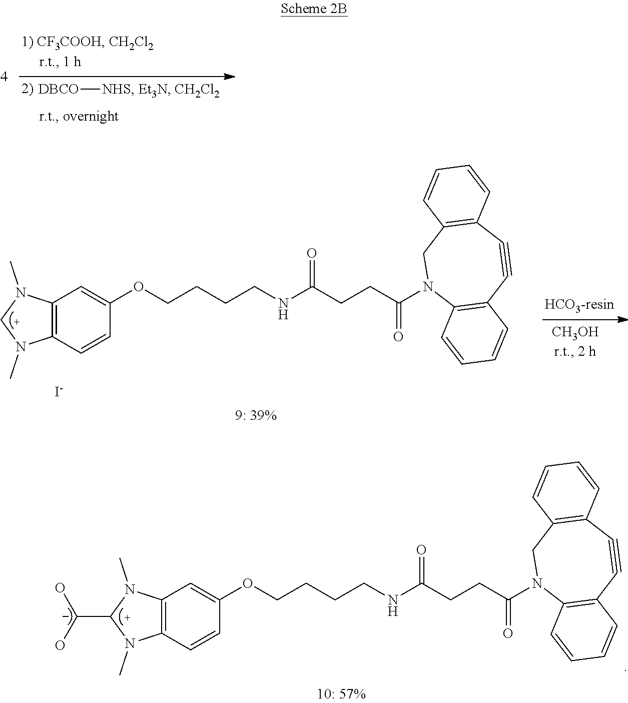

In another aspect, the present disclosure provides a composition comprising an N-heterocyclic carbene (NHC) metal adduct of structural formula III

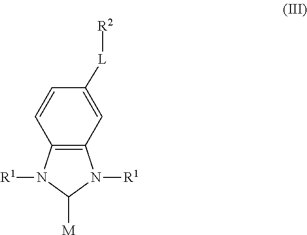

wherein, R¹ is selected from —H, —CH₃, —CH₂—(CH₃)₂, —(C₁-C₆) linear or branched alkyl; R² is a cyclooctyne group; L is a linker comprising a covalently bonded chain of 2 to 100 atoms; and M is an atom of a metal surface.

In at least one embodiment of the NHC metal adduct composition, R¹ is selected from —CH₃, and —CH₂—(CH₃)₂.

In at least one embodiment of the NHC metal adduct composition, the linker L comprises one or more of the following chemical groups: linear (C₁-C₅) alkyl, linear (C₁-C₅) alkenyl, linear (C₁-C₅) alkynyl, ester, ether, amine, amide, imide, phosphodiester, and/or polyethylene glycol (PEG). In at least one embodiment, the linker L comprises a linker of structural formula L_{A}, L_{B}, or L_{C}.

In at least one embodiment of the NHC metal adduct composition, R² is a cyclooctyne group selected from IIa and IIb.

In at least one embodiment of the NHC metal adduct composition, structural formula III is selected from structural formulae IIIa, IIIb, IIIc, IIId, IIIe, IIIf, IIIg, and IIIh:

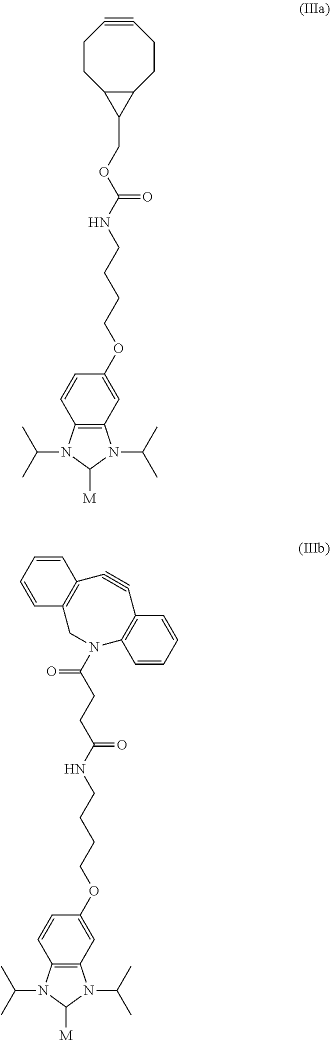

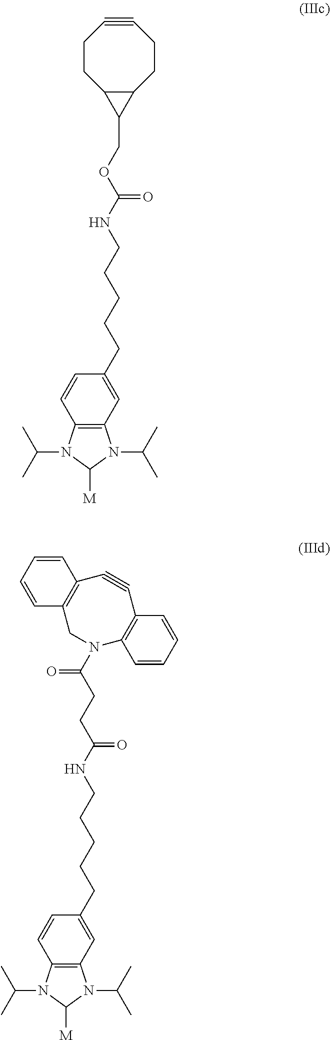

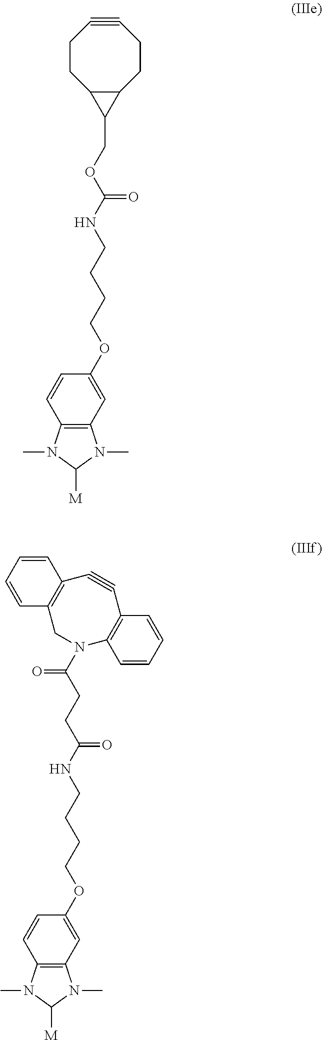

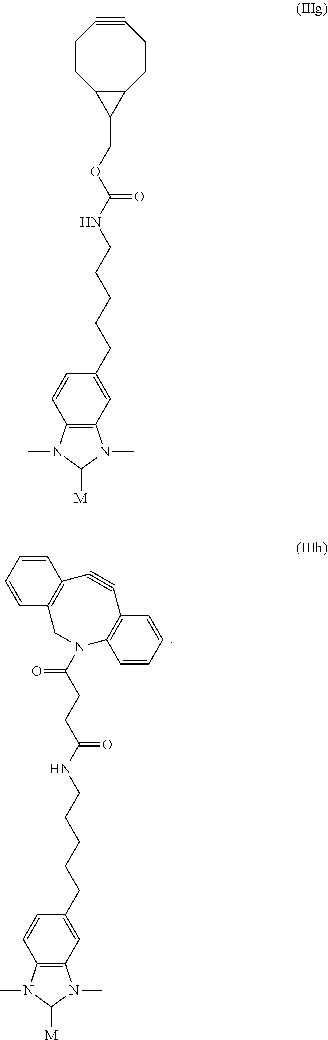

In at least one embodiment of the NHC metal adduct composition, the linkage attaching the N-heterocyclic carbene to the metal surface does not comprise a thiol group.

In at least one embodiment of the NHC metal adduct composition, M is selected from Au, Ag, Pd, Pt, Zn, Cd, and Se.

In at least one embodiment of the NHC metal adduct composition, the metal surface is on a nanoparticle.

In at least one embodiment of the NHC metal adduct composition, metal surface is on an electrode.

In at least one embodiment of the NHC metal adduct composition, the metal surface further comprises a blocking N-heterocyclic carbene metal adduct of structural formula VI

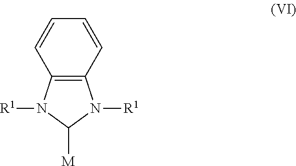

wherein, R¹ is selected from —H, —CH₃, —CH₂—(CH₃)₂, —(C₁-C₆) linear or branched alkyl; and M is an atom of the metal surface. In at least one embodiment, the blocking N-heterocyclic carbene metal adduct of structural formula VI is selected from compounds VIa and VIb

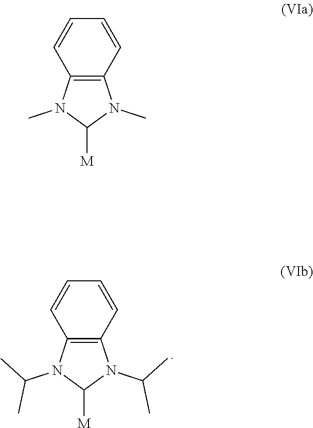

In at least one embodiment of the NHC metal adduct composition, the mole ratio on the surface of the N-heterocyclic carbene metal adduct of structural formula III to the blocking N-heterocyclic carbene metal adduct of structural formula VI is at least about 1:100, at least about 1:250, at least about 1:500, at least about 1:1250, at least about 1:2500, or at least about 1:5000. In at least one embodiment, the mole ratio on the surface of the N-heterocyclic carbene metal adduct of structural formula III to the blocking N-heterocyclic carbene metal adduct of structural formula VI is between about 1:100 and 1:5000, between about 1:250 and 1:2500, or between about 1:500 and 1:1500.

In another aspect, the present disclosure provides a method for preparing a composition comprising an N-heterocyclic carbene metal adduct of structural formula III wherein, R¹ is selected from —H, —CH₃, —CH₂—(CH₃)₂, —(C₁-C₆) linear or branched alkyl; R² is a cyclooctyne group; L is a linker comprising a covalently bonded chain of 2 to 100 atoms; M is an atom of a metal surface; the method comprising: (a) preparing a NHC compound of structural formula I of the present disclosure; and (b) depositing the NHC compound of structural formula I on the metal surface comprising an atom M under vacuum deposition conditions.

In at least one embodiment of the method for preparing a composition comprising an N-heterocyclic carbene metal adduct, M is selected from Au, Ag, Pd, Pt, Zn, Cd, and Se. In at least one embodiment, the metal surface comprising the atom M is on a nanoparticle. In at least one embodiment, the metal surface comprising the atom M is on an electrode.

In another aspect, the present disclosure provides a composition comprising an N-heterocyclic carbene (NHC) metal adduct of structural formula IV

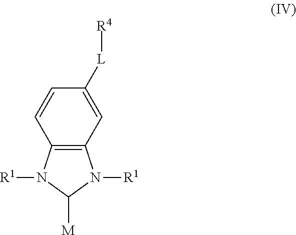

wherein, R¹ is selected from —H, —CH₃, —CH₂—(CH₃)₂, —(C₁-C₆) linear or branched alkyl; L is a linker comprising a covalently bonded chain of 2 to 100 atoms; M is an atom of a metal surface; and R⁴ is a cyclooctyl-triazole adduct comprising structural formula Va or Vb

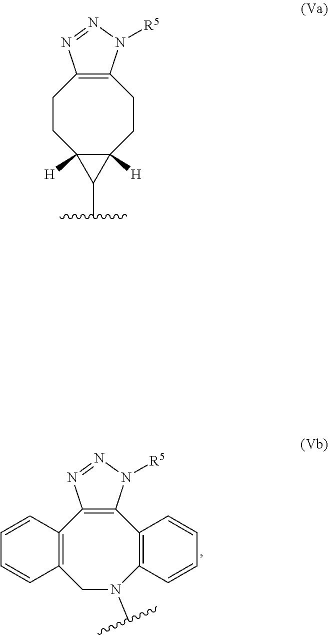

wherein R⁵ comprises a linker moiety covalently attached to a redox-reporter modified aptamer.

In at least one embodiment of the composition comprising an NHC metal adduct of structural formula IV, the aptamer comprises an oligonucleotide. In at least one embodiment, the oligonucleotide is attached to the linker through its 5′-end and attached to the redox-reporter through its 3′-end. In at least one embodiment, the oligonucleotide is attached to the linker through its 3′-end and attached to the redox-reporter through its 5′-end.

In at least one embodiment of the composition comprising an NHC metal adduct of structural formula IV, the redox-reporter comprises a compound selected from methylene blue, thionine, anthraquinone, anthraquinone-C5, Nile blue, neutral red, gallocyanine, dabcyl, 2,6-dichlorophenal-indophenol, ROX, ferrocene, pentamethyl ferrocene, ferrocene-C5, viologen, and Atto MB2.

In at least one embodiment of the composition comprising an NHC metal adduct of structural formula IV, R₁ is selected from —CH₃ and —CH₂—(CH₃)₂.

In at least one embodiment of the composition comprising an NHC metal adduct of structural formula IV, the linker L comprises one or more of the following chemical groups: linear (C₁-C₅) alkyl, linear (C₁-C₅) alkenyl, linear (C₁-C₅) alkynyl, ester, ether, amine, amide, imide, phosphodiester, and/or polyethylene glycol (PEG). In at least one embodiment, L comprises a linker of structural formula L_{A}, L_{B}, or L_{C}.

In at least one embodiment of the composition comprising an NHC metal adduct of structural formula IV, the metal atom M is selected from Au, Ag, Pd, Pt, Zn, Cd, and Se. In at least one embodiment, the metal surface comprising the atom M is on a nanoparticle. In at least one embodiment, the metal surface comprising the atom M is on an electrode. In at least one embodiment, wherein M is Au, the metal surface is on an electrode, and the redox-reporter modified aptamer is an oligonucleotide modified at its 3′-end with methylene blue.

In at least one embodiment of the composition comprising an NHC metal adduct of structural formula IV, formula IV is selected from IVa, IVb, IVc, IVd, IVe, IVf, IVg, and IVh:

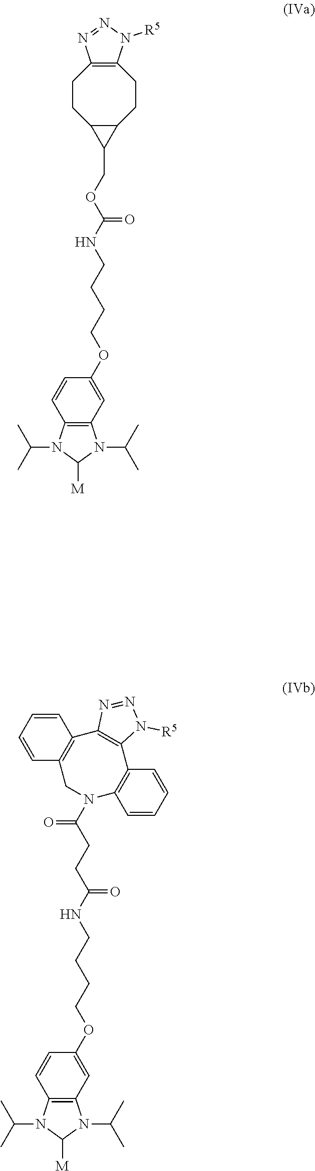

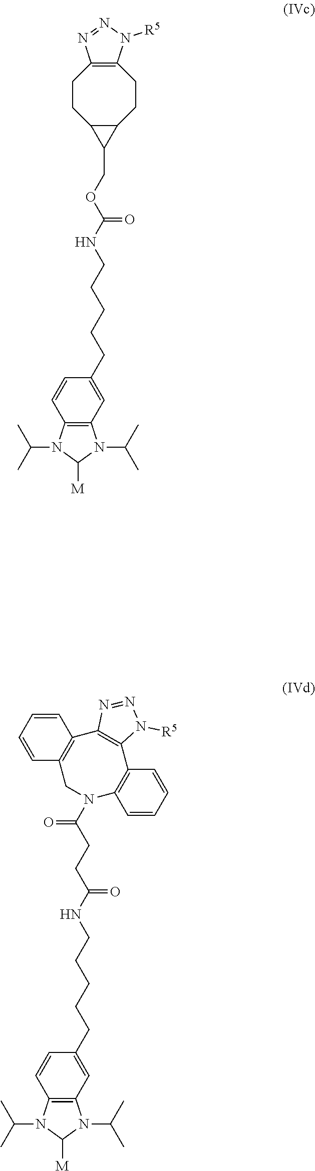

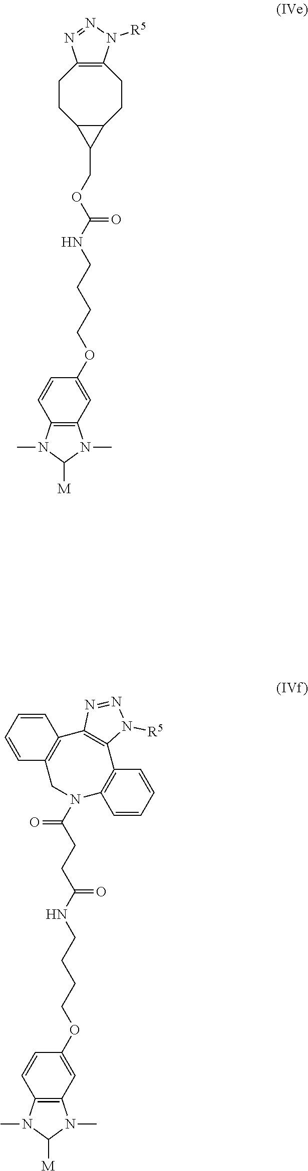

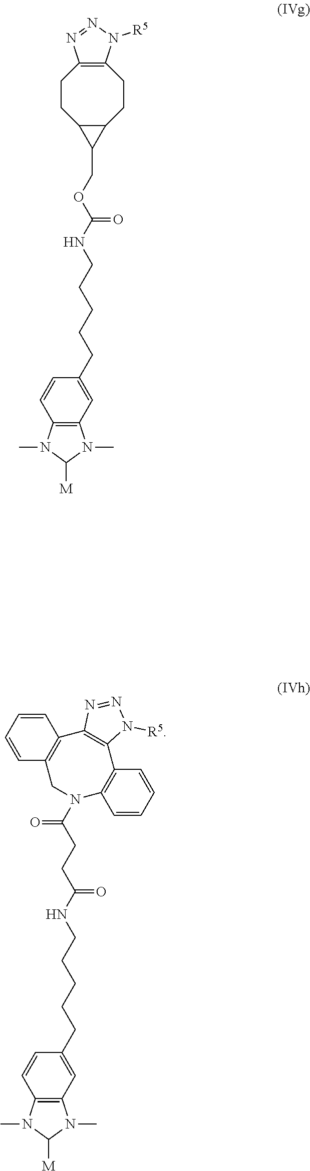

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A and FIG. 1B illustrate how electrochemical aptamer-based sensors (E-ABs) can provide continuous, real-time molecular monitoring in vivo. FIG. 1A: depicts a generalized schematic of a typical thiol monolayer E-AB showing how an aptamer immobilized to the surface can undergo binding-induced changes that affect their electron transfer kinetics. FIG. 1B: depicts a plot of square wave voltammetry measurements from an E-AB sensor showing how aptamer binding induces changes in the electron transfer kinetics to yield a local target concentration which allows for continuous molecular monitoring in vivo.

FIG. 2 depicts a schematic illustration of the rational design of an E-AB sensor that uses a modular N-heterocyclic carbene (NHC) compound as a ligand to attach an aptamer for detection on a metal surface. The left panel illustrates the modular NHC compound ligand chemical properties are tunable via the substituents at the “wing-tip” positions, R, the position R′, and the heterocyclic carbon between the two nitrogen can form strong s-bonds with a variety of surface materials. The right panel illustrates how the NHC modified with a redox-reporter modified aptamer.

FIG. 3 depicts three exemplary cyclooctyne-functionalized NHC compounds 6, 8, and 10 of the present disclosure attached to a gold surface in one exemplary E-AB sensor design of the present disclosure. The cyclooctyne moieties (BCN or DBCO) of the immobilized NHC compounds 6, 8, and 10 can undergo Cu-free strain-promoted azide-alkyne cycloadditions (SPAAC) that release substantial potential energy to facilitate the [3+2]cycloaddition reaction with an azide-modified aptamer compound without a catalyst.

FIG. 4A depicts a process for attaching an exemplary cyclooctyne-functionalized NHC compound of the present disclosure to a gold surface of an electrode and modifying it with an azide-modified aptamer via a Cu-free SPAAC click reaction while attached to the surface.

FIG. 4A depicts vacuum deposition of the BCN-functionalized diisopropyl NHC bicarbonate salt of compound 6 on the surface of a gold electrode followed by catalyst click coupling of the azide-modified aptamer to the BCN group of the surface immobilized NHC compound.

FIG. 4B depicts an alternative process for attaching an exemplary cyclooctyne-functionalized NHC compound of the present disclosure to a gold surface of a nanoparticle.

FIG. 4B depicts a process in which a BCN-functionalized diisopropyl NHC iodide salt of compound 5 is first attached to a gold atom via reaction with AuCl, and then is mixed with gold nanoparticle solution, thereby resulting in a BCN-functionalized NHC compound attached to a gold nanoparticle. The modified nanoparticle can then undergo a further click coupling reaction with an azide-modified aptamer resulting in an aptamer-modified nanoparticle.

FIG. 5A depicts the Cu-free SPAAC click-reaction of the BCN-functionalized diisopropyl NHC bicarbonate salt of compound 6 with 1-hexylazide in solution to form the product compound 11 as described in Example 4A. The numbered spots depicted over certain atoms in the chemical structures of the reacting and product compounds indicate the protons providing the 1H-NMR signals used to monitor the reaction kinetics.

FIG. 5B depicts the 1H-NMR spectra obtained at different time points in the reaction of FIG. 5A and used to measure the reaction kinetics as described in Example 4A. The numbered spots with dashed lines to peaks indicate the 1H-NMR signals corresponding to the protons of the structures of FIG. 5A that were used to monitor the decrease in reactants and increase in the product compound.

FIG. 6A depicts the reaction scheme used in depositing a methanolic solution of the DBCO-functionalized NHC carboxylate adduct of compound 10 and a “blocking” NHC compound 15 in a mole ratio of 1:1250 on the surface of a gold electrode resulting in the metal adduct as described in Example 6B.

FIG. 6B depicts a plot of a square wave voltammogram obtained from the gold electrode prepared with deposited DBCO-functionalized NHC of compound 10 as depicted in FIG. 6A.

FIG. 7A depicts the reaction scheme used in a positive control study described in Example 4B, wherein the “pre-clicked” DBCO-functionalized NHC of compound 14 (a compound that has already undergone the SPAAC click reaction with an azide-modified aptamer to form) and the “blocking” NHC of compound 15 are deposited a 1:1250 mole ratio on a gold electrode.

FIG. 7B depicts a plot of a square wave voltammogram obtained from the gold electrode prepared with deposited “pre-clicked” DBCO-functionalized NHC of compound 14 as depicted in FIG. 7A.

FIG. 8A depicts the reaction scheme used in a negative control study described in Example 4B, wherein only the blocking NHC of compound 15 was deposited on a gold electrode and then a solution of an azide-modified redox-reporter modified aptamer was deposited on the electrode surface overnight at room temperature.

FIG. 8B depicts a plot of a square wave voltammogram obtained from the “negative control” gold electrode prepared with only the blocking NHC of compound 15 and an azide-modified redox-reporter modified aptamer as depicted in FIG. 8A.

FIG. 9A depicts the reaction scheme used to prepare gold electrode surface with co-deposited DBCO-functionalized NHC compound 10 (and blocking compound 15) and then modifying the DBCO-functionalized NHC attached to the electrode with a redox-reporter modified DNA aptamer via SPAAC click reaction as described in Example 4B.

FIG. 9B depicts a plot of a square wave voltammogram obtained from the NHC deposited electrode and modified with an aptamer via a SPAAC click reaction as depicted in FIG. 9A.

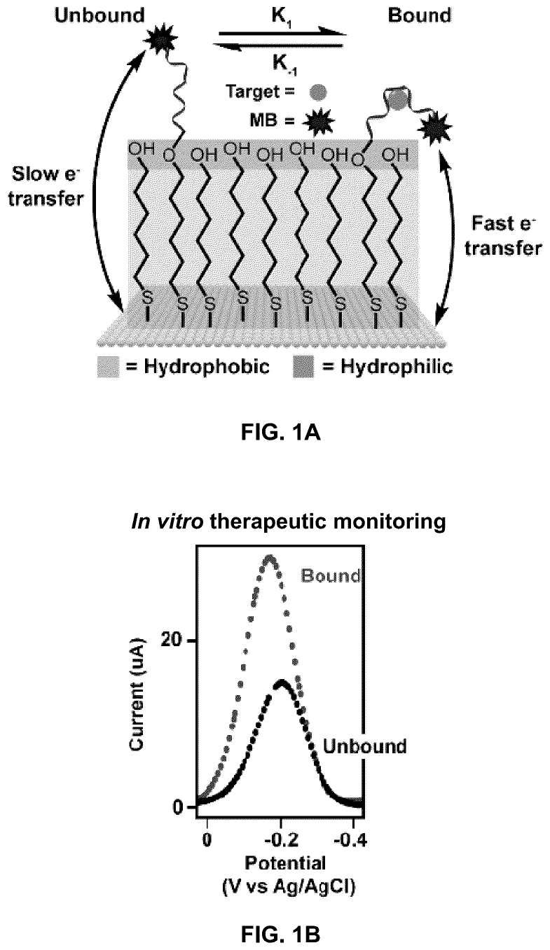

### DETAILED DESCRIPTION

Design of E-AB Sensors with NHC Compounds Monolayers

As noted elsewhere herein, NHC compounds form very strong s-bonds to transition metals, as demonstrated by their ability to form robust self-assembled monolayers on gold surfaces that exhibit greater resilience relative to thiol monolayers when exposed to a wide range of conditions. NHC monolayers, for example, can resist chemical degradation, enable post-synthetic modification of the NHC monolayer, support electrochemical detection, and tolerate exposure under competitive displacement conditions compared to physiologically thiol molecules. Thus, NHC compounds are capable of overcoming two failure mechanisms common to thiol-monolayers: passive desorption and competitive displacement.

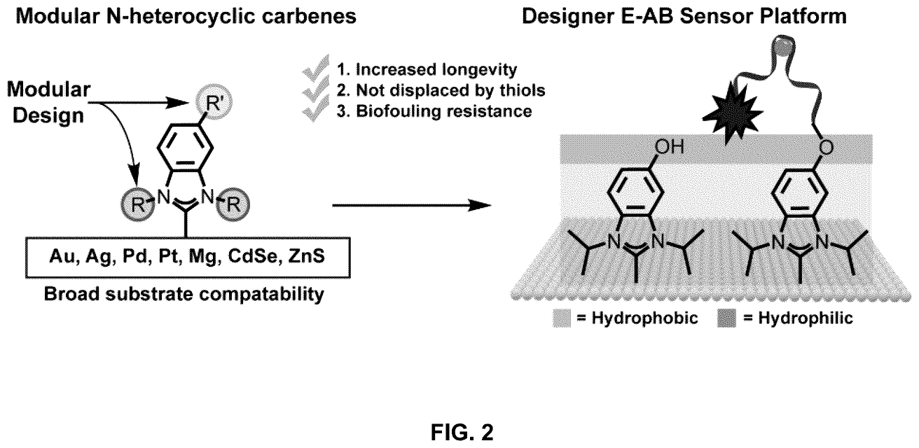

FIG. 2 depicts a schematic illustration of an exemplary E-AB sensor design of the present disclosure that incorporates an NHC compound monolayer. The NHC compound is modular and comprises positions allowing covalent attachment to the metal surface, covalent attachment to an aptamer, and positions that allow for tuning of the packing of the NHC compounds on the surface. The left panel of FIG. 2 illustrates the modular structure of the NHC compound including tunable via “wing-tip” group, R, and a backbone group, R′, that allows attachment of an aptamer ligand. The carbon located between the nitrogen atoms of the heterocyclic ring can form strong s-bonds with the metal atoms in a variety of surface materials, including but not limited to Au, Ag, Pd, Pt, Mg, CdSe, and ZnS. The modularity of the NHC compound monolayer illustrated in FIG. 2 allows for a rational E-AB design that can overcome the failure mechanisms found in typical thiol monolayer E-AB designs. It is contemplated that rational design of an NHC compound to incorporate substituents known to prevent protein binding is possible and could lead to mitigation of E-AB biofouling.

### Design and Synthesis of Cyclooctyne-Functionalized NHC Compounds

Bioorthogonal click chemistry reactions have revolutionized biochemistry due to their high yield, fast kinetics, and activity at room temperature in biofluids (59-61). Multiple researchers have prepared NHCs with an azide functional group on the backbone or wingtips (52, 62, 63) which have led to successful copper(I)-catalyzed azide-alkyne click (CuAAC) reactions. Copper catalysts, however, can result in cytotoxicity for living cells, which restricts in vivo applications (60,64). Unlike conventional CuAAC click reactions, strain-promoted azide-alkyne cycloadditions (SPAAC) employ the reaction of azides with strained-ring alkynes, such as bicyclo[6.1.0]nonyne (BCN) and dibenzoazacyclooctyne (DBCO), that release substantial potential energy to facilitate the [3+2]cycloaddition reaction without a Cu catalyst (60, 61, 65).

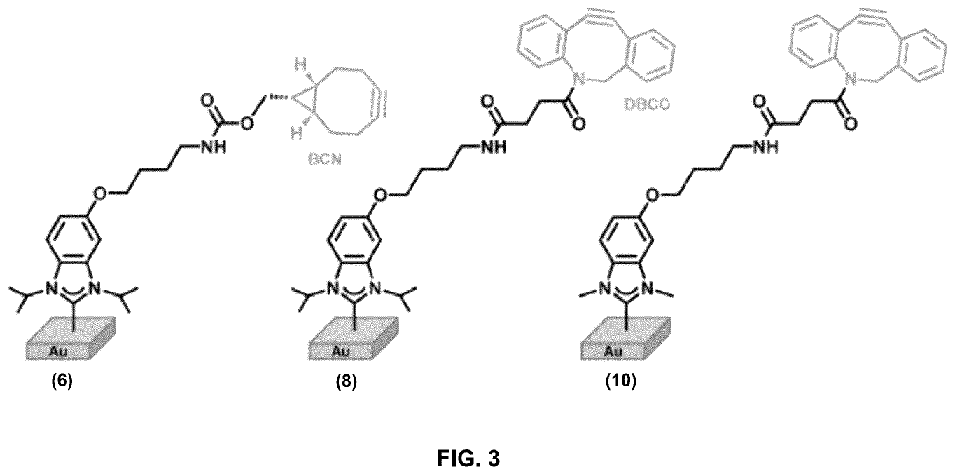

The present disclosure provides cyclooctyne-functionalized NHC compounds that can be used as monolayer-forming ligands in the construction of E-AB and allow attachment to the E-AB surface via a SPAAC click reaction of a wide array of azide-modified redox-reporter modified aptamers. The NHC are functionalized with cyclooctyne groups known undergo facile Cu-free SPAAC click reactions, including but not limited to, bicyclo[6.1.0]nonyne (“BCN”) and dibenzoazacyclooctyne (“DBCO”). FIG. 3 shows three exemplary cyclooctyne-functionalized NHC compounds of the present disclosure denoted compounds 6, 8, and 10, in a schematic illustration also showing their attachment to a gold surface, such as an electrode. The surface attached NHC group is attached to the cyclooctyne groups (BCN or DBCO) through an exemplary 10-atom linker group.

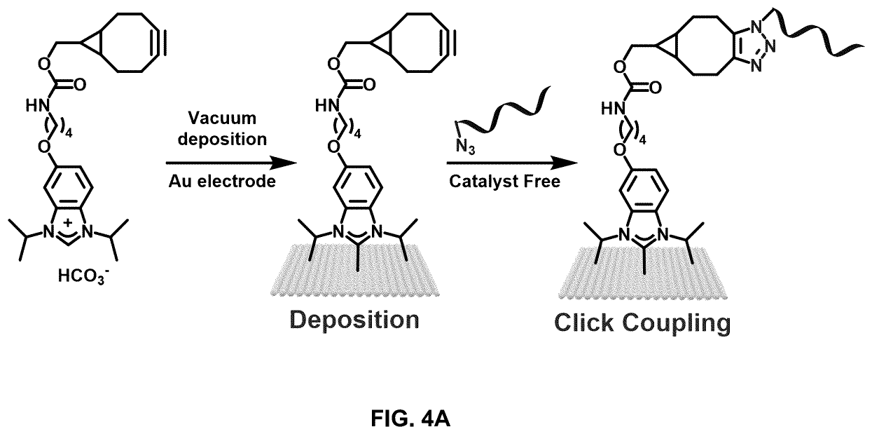

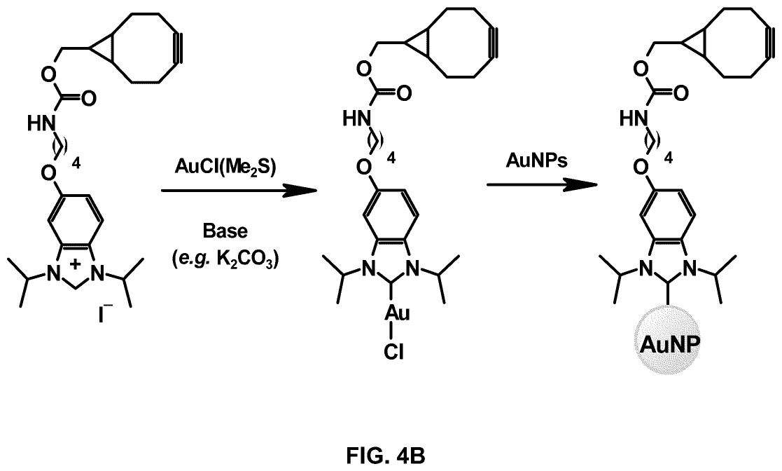

The BCN and DBCO groups of these surface-attached NHC compounds are capable of undergoing Cu-free SPAAC reaction with an azide-modified aptamer compound. FIG. 4A depicts a general process of attaching the exemplary BCN-functionalized diisopropyl “wing-tip” NHC design of compound 6 on a surface of an Au electrode via standard vacuum deposition techniques. The resulting covalently surface-attached NHC is then reacted with an azide-modified aptamer via a Cu-free SPAAC click reaction to form the aptamer-modified NHC metal-adduct with the metal of the electrode surface. An extensive library of azide-functionalized aptamers (e.g., DNA or RNA oligonucleotides) are known and commercially available. Besides metal surfaces of electrodes, as depicted in FIG. 4B, it is also contemplated that the cyclooctyne-functionalized NHCs of the present disclosure can be attached to the metal surfaces of nanoparticles and undergo SPAAC reaction on the nanoparticle to attach any azide-modified biomolecule to the nanoparticle. Accordingly, it is contemplated that the NHC compounds of the present disclosure can be used in applications that employ biomolecule-modified nanoparticles including but not limited to spectroscopic techniques, mass spectrometry, and drug delivery.

Generally, the present disclosure provides cyclooctyne-functionalized NHC compounds of structural formula I

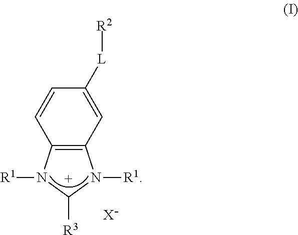

In this structure, the “wing-tip” positions of the NHC heterocycle denoted as R¹ are groups that include —H, —CH₃, —CH₂—(CH₃)₂, and —(C₁-C₆) linear or branched alkyl. The R² group is the cyclooctyne group. The linker group, L can be any covalently bonded chain of 2 to 100 atoms. The R³ group is attached to the carbon between the two nitrogen atoms of the NHC heterocycle and can be —H and —COO⁻. In some embodiments, the presence of the carboxylate (—COO⁻) at the R³ position can be used to facilitate formation of a metal adduct. Alternatively, when R³ is —H, the presence of an anion denoted X⁻ in the structure offsets the positively charged NHC heterocycle.

A range of cyclooctyne-functionalized NHC compounds of structural formula I are contemplated within the scope of the present disclosure, including, but not limited to embodiments having the following structural features alone or in combination:

The group at position R¹ is selected from —CH₃ and —CH₂—(CH₃)₂.

The linker L comprises one or more of the following chemical groups: linear (C₁-C₅) alkyl, linear (C₁-C₅) alkenyl, linear (C₁-C₅) alkynyl, ester, ether, amine, amide, imide, phosphodiester, and/or polyethylene glycol (PEG); or the linker L comprises a linker of structural formula L_{A}, L_{B}, or L_{C}

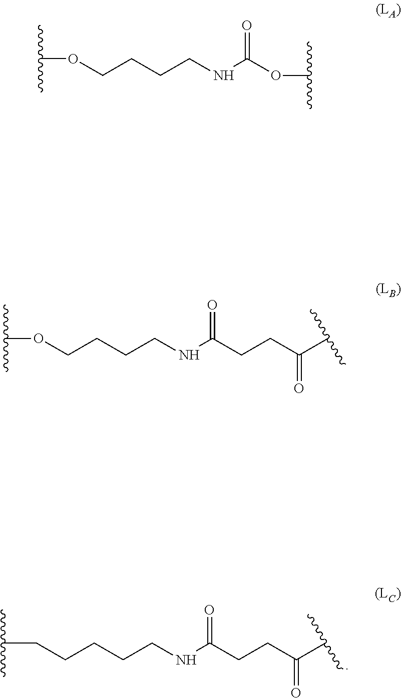 [SMILES: *OCCCCNC(=O)O* ; *OCCCCNC(=O)CCC(*)=O ; *CCCCCNC(=O)CCC(*)=O]

The group R² is a cyclooctyne moiety selected from IIa (BCN) and IIb (DBCO):

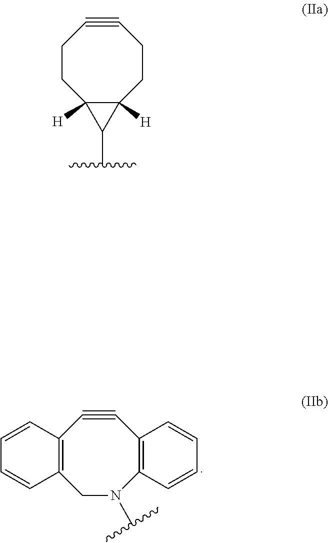 [SMILES: *C1[C@H]2CCC#CCC[C@@H]12 ; *N1Cc2ccccc2C#Cc2ccccc21]

The group R³ is and —H and the anion, X⁻ is a halide or HCO₃⁻; optionally, when X⁻ is a halide, the halide can be F⁻, Cl⁻, Br⁻, or I⁻; or the group R³ can —COO⁻ without an anion X⁻ present.

Accordingly, among the exemplary cyclooctyne-functionalized NHC compounds of structural formula I contemplated by the present disclosure are the compounds of structural formulae Ia, Ib, Ic, Id, Ie, If, Ig, and Ih shown below.

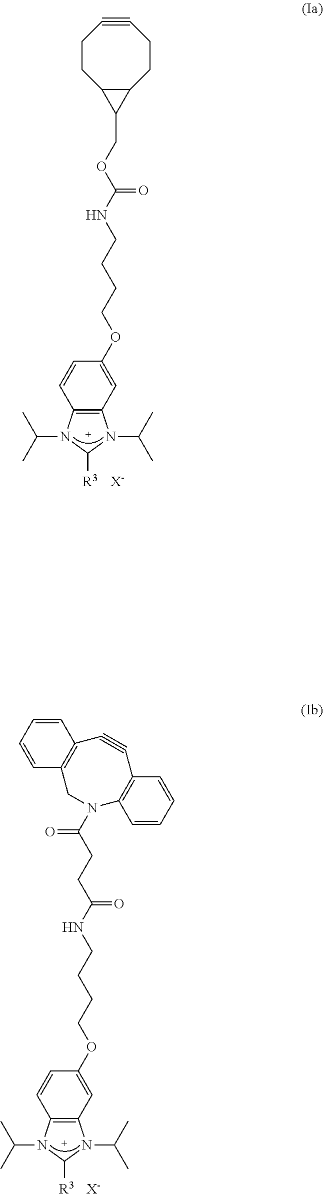

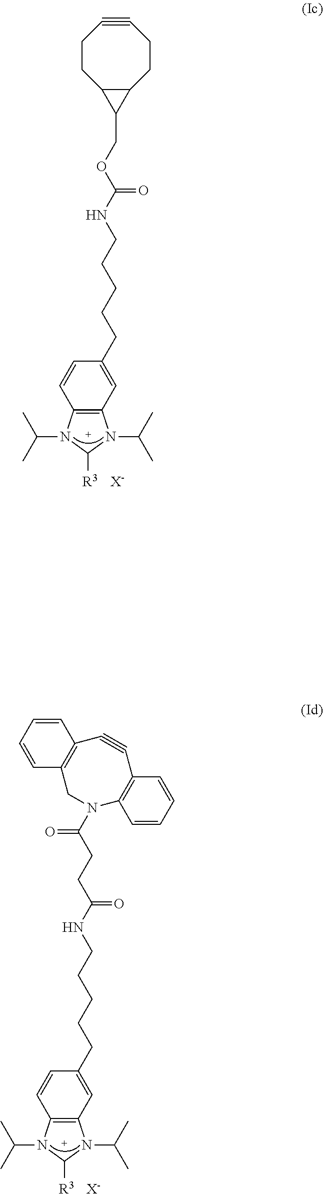

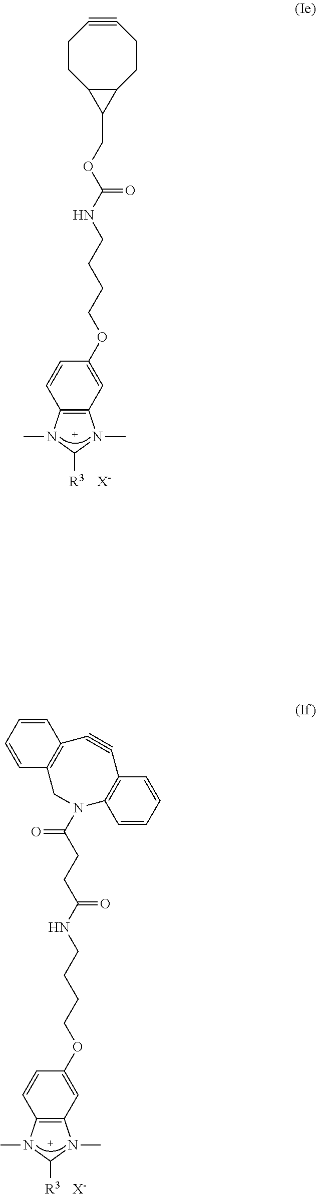

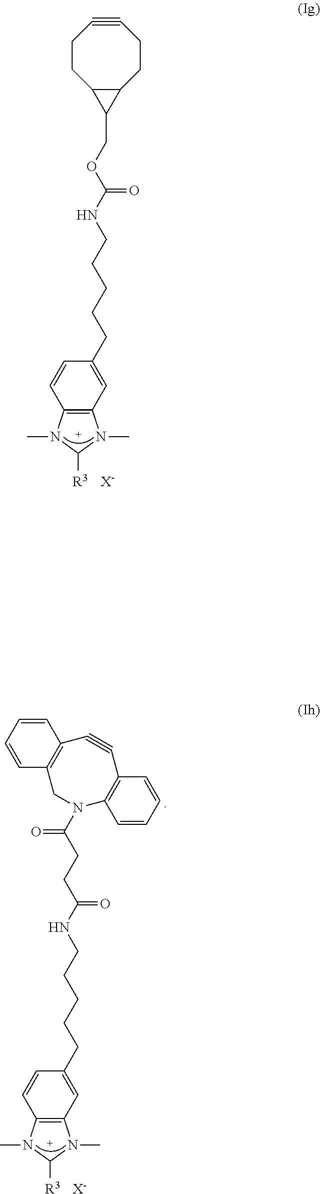

Specific exemplary cyclooctyne-functionalized NHC compounds of structural formula I contemplated by the present disclosure are the compounds 5, 6, 7, 8, 9, and 10 shown below.

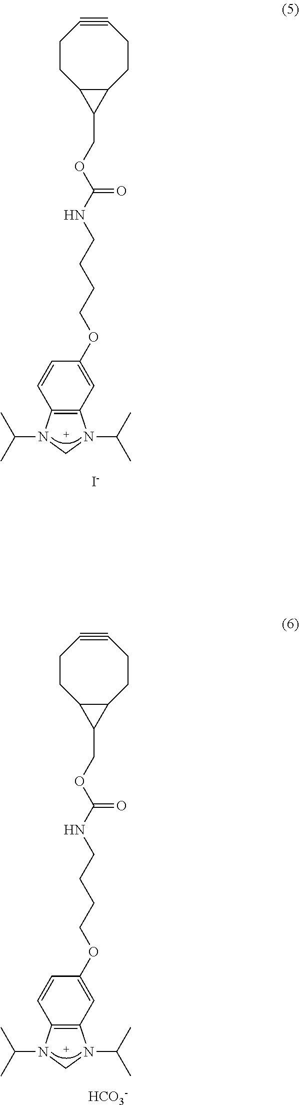

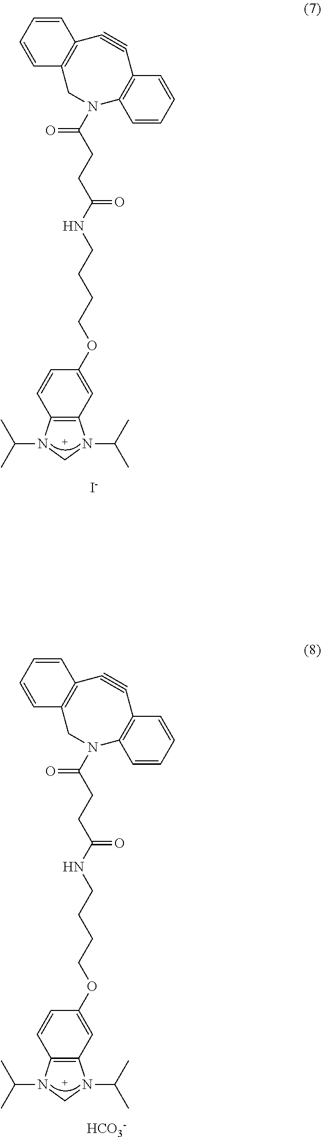

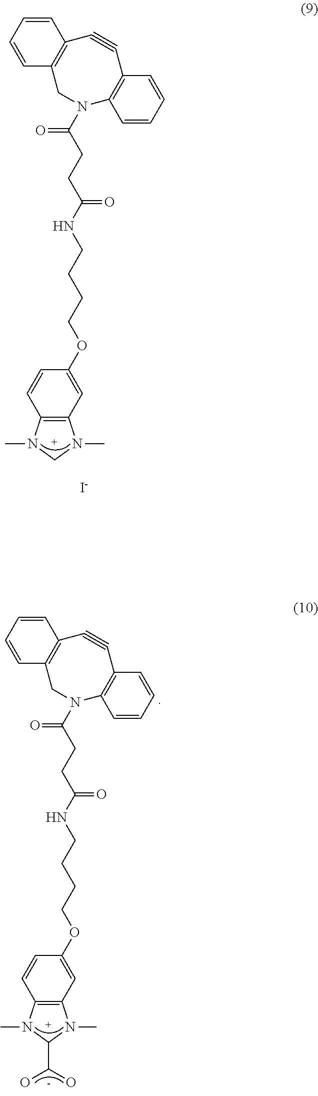

### Synthesis of Cyclooctyne-Functionalized NHC Compounds

As described in the Examples and elsewhere herein, the cyclooctyne-functionalized NHC compounds of structural formula I can be prepared via two different synthetic strategies to attach the strained cyclooctyne moieties to the NHC group: (1) via a Boc-protected precursor that forms a linker with an amide bond; or (2) via a Boc-protected precursor that forms linker with a carbamate. Once the Boc-protected precursor is synthesized, the synthesis of the cyclooctyne-functionalized NHC compounds can be carried out via a three-step procedure, which includes the steps of Boc deprotection, carbamate ester or amide coupling, and anion exchange. Iodide salt intermediates were prepared with anion exchange used to produce the bicarbonate salts which are known to be highly effective for placing NHC on gold surfaces under vacuum with modest heating (48, 62, 66-69).

The preparation of the Boc-protected precursor compounds (e.g., compounds 3 or 4) can be carried out according to the 3-step synthesis of Scheme 1. It will be noted that the “wing-tip” position groups, R, are selected by the choice of iodide reagent used in the third step reaction. As shown in the exemplary Scheme 1, precursor compounds with isopropyl and methyl “wing-tip” groups, compounds 3 and 4, respectively can be synthesized with essentially the same procedure.

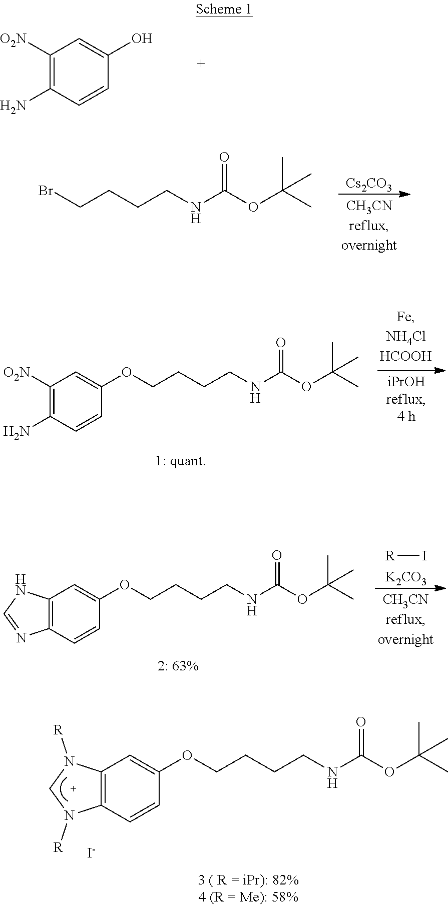

The Boc-protected precursor compound 3 of Scheme 1 with isopropyl “wing-tip” groups can then be used to prepare a BCN-functionalized NHC compound with a carbamate linker and isopropyl “wing-tip” groups (e.g., compounds 5, and 6) or a DBCO-functionalized NHC compound with an amide linker and isopropyl “wing-tip” groups (e.g., compounds 7 and 8) according to the 3-step synthesis of Scheme 2A. The commercially available reagents, BCN—NHS and DBCO—NHS used at step 2 determine the specific cyclooctyne group and type of linkage (carbamate or amide) to the NHC group of the precursor.

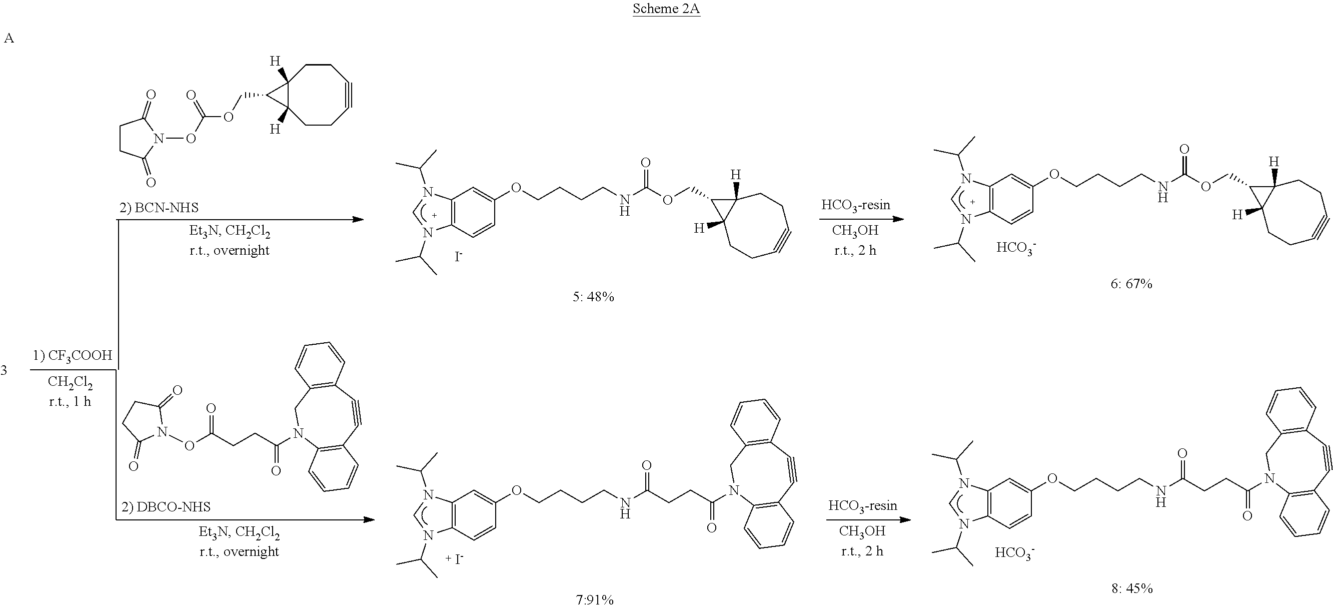

Alternatively, the Boc-protected precursor compound 4 of Scheme 1 with methyl “wing-tip” groups can be used to prepare DBCO-functionalized NHC compound with an amide linker and methyl “wing-tip” groups (e.g., compounds 9 or 10) according to the 3-step synthesis of Scheme 2B.

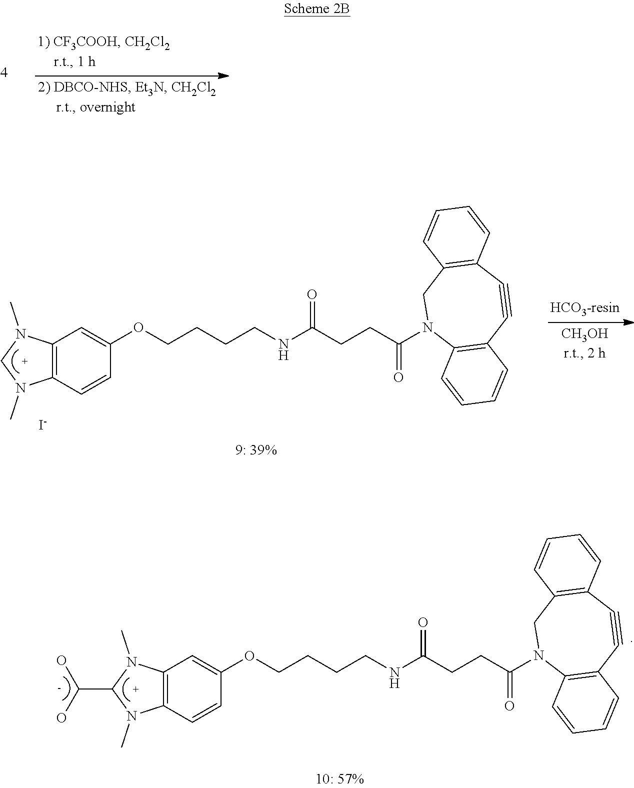 [SMILES: CN1CN(C)c2cc(OCCCCNC(=O)CCC(=O)N3Cc4ccccc4C#Cc4ccccc43)ccc21 ; [I-] ; CN1c2ccc(OCCCCNC(=O)CCC(=O)N3Cc4ccccc4C#Cc4ccccc43)cc2N(C)C1C(O)O]

The methods of synthesis described above can be used in preparing any cyclooctyne-functionalized N-heterocyclic carbene (NHC) compound of structural formula I of the present disclosure. In at least one embodiment, the method can be used to prepare a compound of structural formula I wherein, R¹ is selected from —CH₃, and —CH₂—(CH₃)₂; R² is a cyclooctyne group selected from IIa and IIb; L comprises a linker of structural formula L_{A} or L_{B}; R³ is selected from —H and —COO⁻; and X⁻ is an optional anion that is present when R³ is —H, including but not limited to of compound 5, 6, 7, 8, 9 or 10.

In at least one embodiment, the method can be used to produce the BCN-functionalized compounds 5 and 6, wherein the method comprises the steps of: (a) preparing a Boc-protected precursor compound 3 according to the synthesis steps of Scheme 1; and (b) reacting the precursor compound 3 of step (a) with BCN—NHS according to the synthesis steps of Scheme 2A to prepare a BCN-functionalized NHC iodide salt of compound 5; and optionally, exchanging the iodide of compound 5 with HCO₃⁻ by anion exchange to produce the bicarbonate salt of compound 6.

In at least one embodiment, the method can be used to produce the DBCO-functionalized compounds 7 and 8, wherein the method comprises the steps of: (a) preparing a Boc-protected precursor compound 3 according to the synthesis steps of Scheme 1; and (b) reacting the precursor compound 3 of step (a) with DBCO—NHS according to the synthesis steps of Scheme 2A to prepare a DBCO-functionalized NHC iodide salt of compound 7; and optionally, exchanging the iodide of compound 5 with HCO₃⁻ by anion exchange to produce the bicarbonate salt of compound 8.

In at least one embodiment, the method can be used to produce the DBCO-functionalized of compounds 9 and 10 with methyl “wing-tip” groups, wherein the method comprises the steps of: (a) preparing a Boc-protected precursor compound 4 according to the synthesis steps of Scheme 1; and (b) reacting the precursor compound 4 of step (a) with DBCO—NHS according to the synthesis steps of Scheme 2B to prepare a DBCO-functionalized NHC iodide salt of compound 9; and optionally, exchanging the iodide of compound 9 with HCO₃ by anion exchange to produce the carboxylate NHC of compound 10.

The present disclosure also contemplates a cyclooctyne-functionalized NHC that does not include an ether group in the linker the NHC moiety and the cyclooctyne group. Such a linker would likely be more resistant to chemical or electrical oxidation under certain use conditions. Accordingly, in at least one embodiment instead of utilizing the synthetic steps found in Schemes 1, 2A and 2B, a cyclooctyne-functionalized NHC that does not include an ether group in the linker between the can be prepared according to the synthetic steps of Scheme 4.

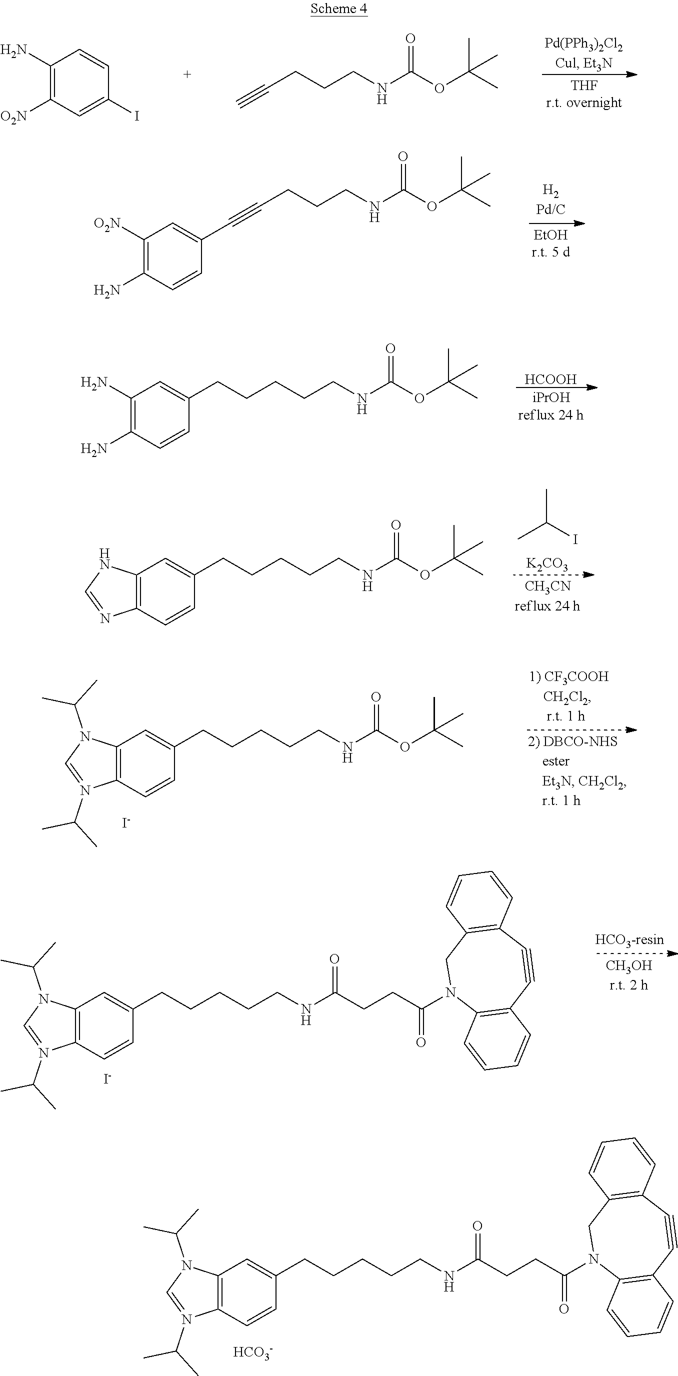

Although Scheme 4 depicts the use of a DBCO—NHS to form the cyclooctyne, it is contemplated that BCN—NHS (or other useful cyclooctyne-NHS groups) could be used in the synthesis method based on Scheme 4.

Additional details of the synthetic methods useful in preparing a cyclooctyne-functionalized N-heterocyclic carbene (NHC) compound of structural formula I of the present disclosure, including the exemplary compounds 5, 6, 7, 8, 9, and 10, are provided in the Examples.

### Preparation of Cyclooctyne-Functionalized NHC Metal-Adducts

As described elsewhere herein, the cyclooctyne-functionalized NHC compounds of structural formula I of the present disclosure are designed to form an adduct (e.g., a covalent s-bond) between a carbon of the NHC moiety and an atom of a metal surface, such as an Au atom on the surface of an electrode. Formation of the NHC-metal adduct can then be followed by a Cu-free SPAAC click reaction with an azide modified biomolecule (e.g., an aptamer). This general process is illustrated schematically in FIG. 4A which shows attaching an exemplary BCN-functionalized NHC design of compound 6, which is a bicarbonate salt, to a surface of an Au electrode. As described in the Examples, standard vacuum deposition techniques at an elevated temperature (e.g., 55 C) can be used to form a covalent s-bond between a gold atom of the surface and the carbon located on the NHC moiety between the two N atoms. As described elsewhere herein including the Examples, the resulting covalently surface-attached NHC can then be reacted under typical Cu-free SPAAC click reaction conditions with an azide-modified aptamer (or any other azide-modified biomolecule) to form the aptamer-modified NHC metal-adduct attached to the electrode surface.

Accordingly, in at least one embodiment, the present disclosure provides a cyclooctyne-functionalized NHC metal-adduct of structural formula III

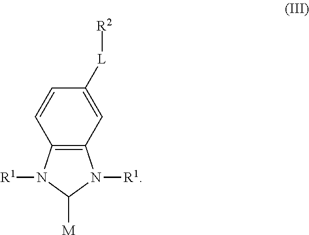

The cyclooctyne-functionalized NHC metal-adducts of structural formula III can have the same “wing-tip” groups at position R¹, cyclooctyne groups at position R², and any of the same linker L moieties as described herein for the cyclooctyne-functionalized NHC compound of structural formula I.

As noted elsewhere herein, it is generally preferred that the linkage attaching the N-heterocyclic carbene to the metal surface does not include a thiol group.

The metal atom, M, of the structural formula III can be selected from any metal that is able to form a strong covalent bond to the NHC moiety, including but not limited to Au, Ag, Pd, Pt, Zn, Cd, and Se. Generally, the metal atom, M, is part of a larger metal surface. For example, the NHC metal adduct composition can comprise a metal surface on an electrode.

As noted elsewhere herein, it is also contemplated that the atom M is part of metal surface on a nanoparticle.

The NHC metal adduct compositions of structural formula III include but are not limited to the compositions of structural formulae IIIa, IIIb, IIIc, IIId, IIIe, IIIf, IIIg, and IIIh:

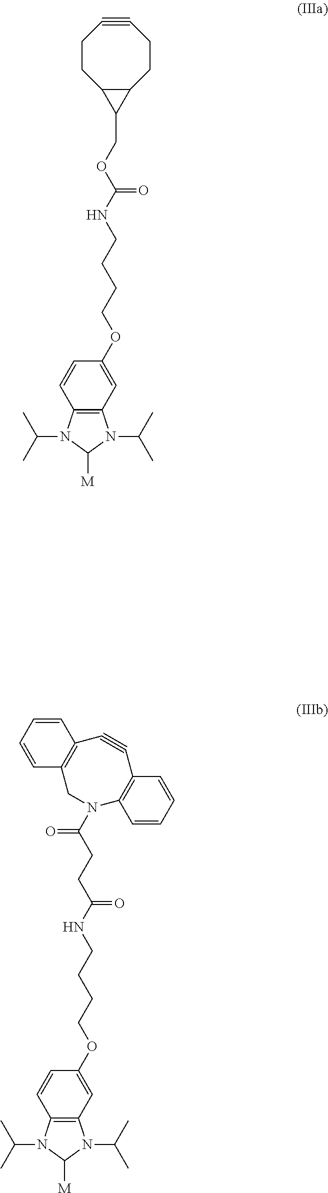

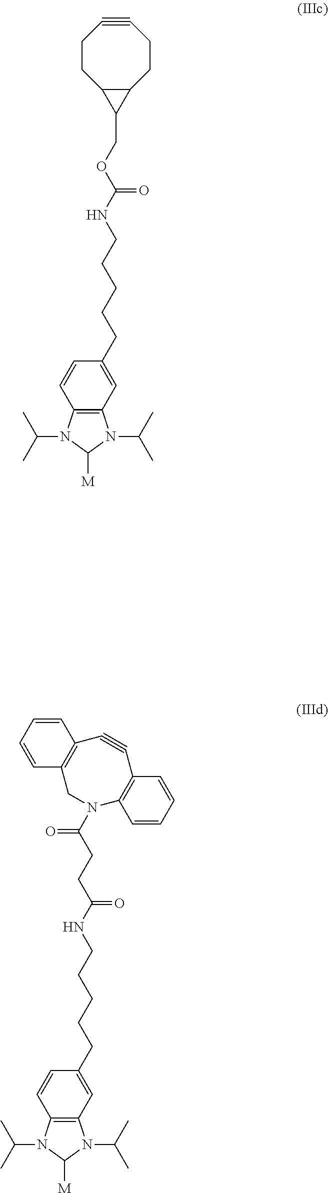

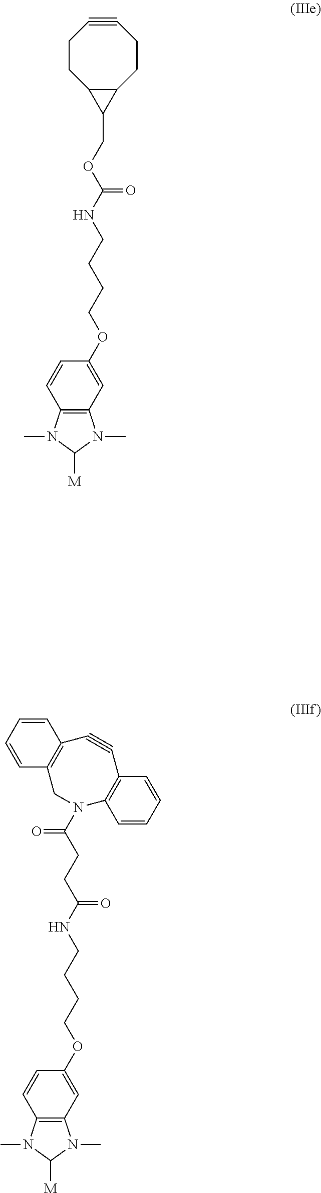

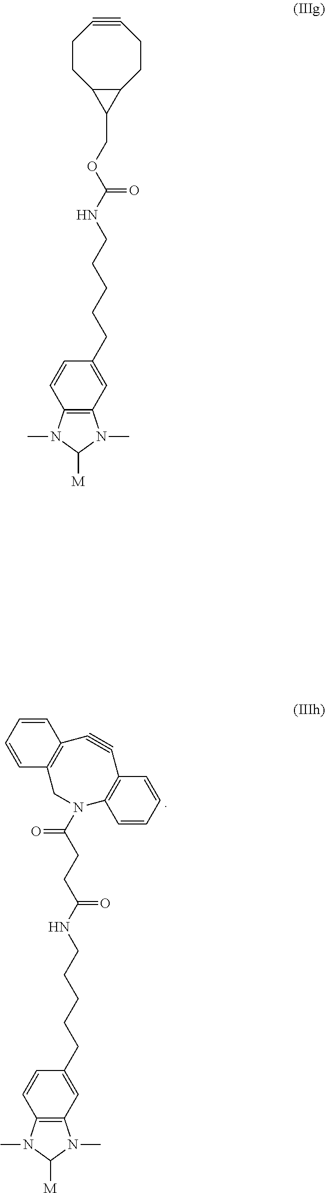

As described elsewhere herein, including the Examples, the NHC metal adduct compositions on a metal surface of the present disclosure are often used in the presence of a different “blocking” NHC metal adduct compound on the surface. Structurally, the blocking NHC compounds do not any cyclooctyne functional group modification of the aryl ring of the NHC moiety, but are otherwise structural analogs of the cyclooctyne-functionalized compounds of the present disclosure (e.g., NHC compounds of structural formula I). The blocking NHC compounds are easily mixed with the cyclooctyne-functionalized NHC compound and thus capable of forming a covalently attached monolayer on the same metal surface. The presence of the blocking NHC metal adduct compounds on the surface thereby provide spacing in the monolayer between the cyclooctyne-functionalized NHC metal adducts (e.g., structural formula III). This spacing can be tuned by adjusting the mole ratio of the blocking NHC to the cyclooctyne-functionalized NHC compounds in the mixture applied to the surface, and thereby adjusted to provide improved kinetics of the SPAAC reaction with the cyclooctyne groups, and also improved binding of target molecules to an aptamer or biomolecule that is attached to the NHC metal adduct, thereby resulting in improved biosensor performance. The use of and tuning of blocking compounds on surfaces of sensors such as E-ABs, is known in the art, and exemplary methods using blocking NHC compounds is described in the Examples of the present disclosure.

Accordingly, in at least one embodiment, the present disclosure contemplates that the metal surface of NHC metal adduct composition of structural formula III can further comprise a blocking N-heterocyclic carbene metal adduct of structural formula VI

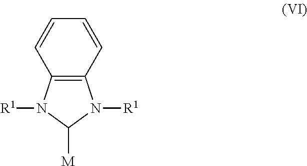

wherein, R¹ is selected from —H, —CH₃, —CH₂—(CH₃)₂, —(C₁-C₆) linear or branched alkyl; and M is an atom of the metal surface. In at least one embodiment, the blocking N-heterocyclic carbene metal adduct of structural formula VI is selected from compounds VIa and VIb

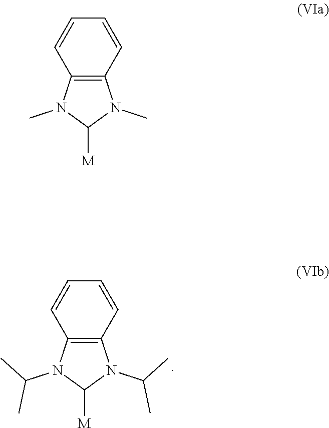

As described above and elsewhere herein, the spacing of the blocking NHC compound to the cyclooctyne-functionalized NHC on the metal surface can be adjusted to improve performance as a biosensor. As described in the Examples, this spacing can be adjusted by adjusting the structure of the blocking compound and/or by adjusting the mole ratio of the cyclooctyne-functionalized NHC to the blocking NHC compound in the mixture of the solution applied to the surface during the deposition process. Accordingly, in at least one embodiment of the NHC metal adduct composition, the mole ratio on the surface of the N-heterocyclic carbene metal adduct of structural formula III to the blocking N-heterocyclic carbene metal adduct of structural formula VI is at least about 1:100, at least about 1:250, at least about 1:500, at least about 1:1250, at least about 1:2500, or at least about 1:5000. In at least one embodiment, the mole ratio on the surface of the N-heterocyclic carbene metal adduct of structural formula III to the blocking N-heterocyclic carbene metal adduct of structural formula VI is between about 1:100 and 1:5000, between about 1:250 and 1:2500, or between about 1:500 and 1:1500.

As described elsewhere herein, one of the advantages of the cyclooctyne-functionalized NHC of structural formula I when they are attached to a surface and form a metal adduct composition of structural formula III, is that they can be easily modified via a SPAAC click reaction with an azide-modified biomolecule. See e.g., FIG. 4A and the Examples. Accordingly, the present disclosure also contemplates N-heterocyclic carbene (NHC) metal adduct composition of structural formula IV which is formed on a metal surface after undergoing the SPAAC reaction with azide-modified biomolecule.

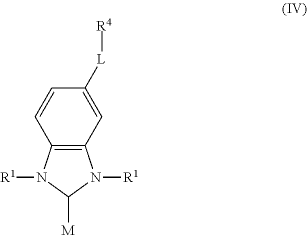

The cyclooctyne-functionalized NHC metal-adducts of structural formula IV can have the same “wing-tip” groups at position R¹, and any of the same linker L moieties as described herein for the cyclooctyne-functionalized NHC compound of structural formula I. Similarly, the metal atom, M, and the associated metal surfaces are as described herein for the cyclooctyne-functionalized NHC metal-adduct of structural formula III.

It should be noted, however, that the composition of structural formula IV does not include the R² group found in the structure of formulae I or III, but rather has a group R⁴ that represents the cyclooctyl-triazole adduct formed in the SPAAC reaction between the cyclooctyne and the azide modified biomolecule. Accordingly, the group R⁴ can be a moiety of structural formula Va, derived from the SPAAC reaction of the BCN cyclooctyne with an R⁵-azide compound, or a moiety of structural formula Vb, derived from reaction of the DBCO cyclooctyne with an R⁵-azide compound.

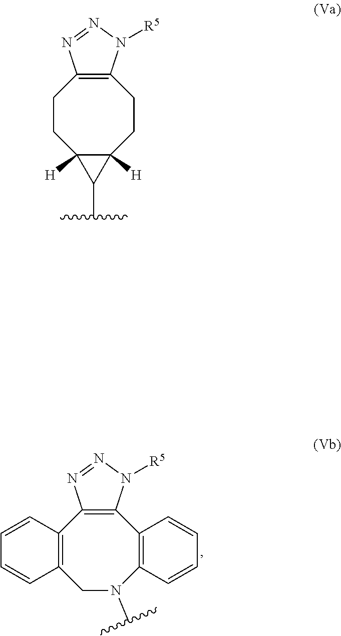

In at least one embodiment, the group R⁵ comprises a linker and a biomolecule. In at least one embodiment, the biomolecule is labeled with a moiety capable of generating a detectable signal, such as a redox-reporter moiety or a fluorescent moiety. In at least one embodiment, the group R⁵ comprises a redox-reporter modified aptamer. A wide range of redox-reporter molecules useful in modifying aptamers and other biomolecules and useful in the compositions and sensor applications of the present disclosure are known in the art and include e.g., methylene blue, thionine, anthraquinone, anthraquinone-C5, Nile blue, neutral red, gallocyanine, dabcyl, 2,6-dichlorophenal-indophenol, ROX, ferrocene, pentamethyl ferrocene, ferrocene-C5, viologen, and Atto MB2. See e.g., Kang et al., “Survey of Redox-Active Moieties for Application in Multiplexed Electrochemical Biosensors,” Anal Chem. 2016 Oct. 11; 88(21):10452-10458. doi: 10.1021/acs.analchem.6b02376;

In at least one embodiment of the composition comprising an NHC metal adduct of structural formula IV, the group R⁵ comprises an aptamer that is an oligonucleotide. The oligonucleotide can comprise naturally occurring bases and linkages (e.g., DNA, RNA), and/or any of the synthetic monomer units and/or linkages used as aptamers that are known in the art.

In at least one embodiment, the group R⁵ comprises a redox-reporter modified aptamer that is oligonucleotide attached to a linker through its 5′-end and attached to the redox-reporter through its 3′-end. In another embodiment, the group R⁵ comprises a redox-reporter modified aptamer that is oligonucleotide attached to a linker through its 3′-end and attached to the redox-reporter through its 5′-end. In at least one embodiment of the composition comprising an NHC metal adduct of structural formula IV, the redox-reporter comprises a moiety selected from methylene blue, thionine, anthraquinone, anthraquinone-C5, Nile blue, neutral red, gallocyanine, dabcyl, 2,6-dichlorophenal-indophenol, ROX, ferrocene, pentamethyl ferrocene, ferrocene-C5, viologen, and Atto MB2. In at least one embodiment of the composition comprising an NHC metal adduct of structural formula IV, M is an Au atom of a metal surface on an electrode, and R⁵ comprises a redox-reporter modified aptamer is an oligonucleotide modified at its 3′-end with methylene blue.

The NHC metal adduct compositions of structural formula IV include but are not limited to the compositions of structural formulae IVa, IVb, IVc, IVd, IVe, IVf, IVg, and IVh:

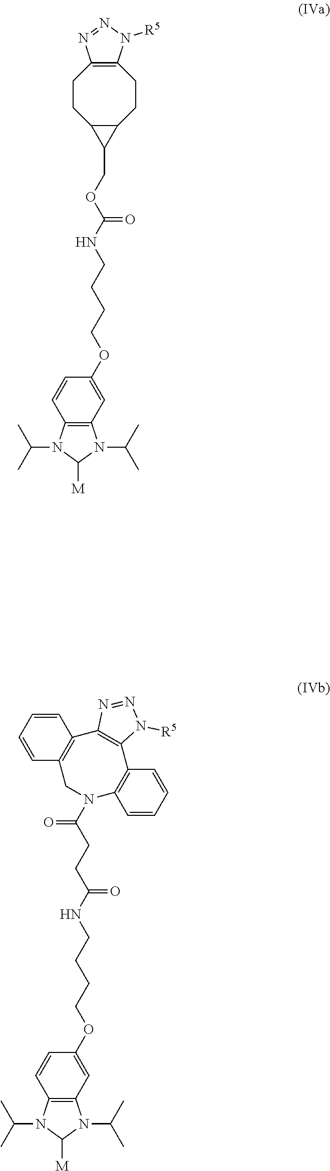

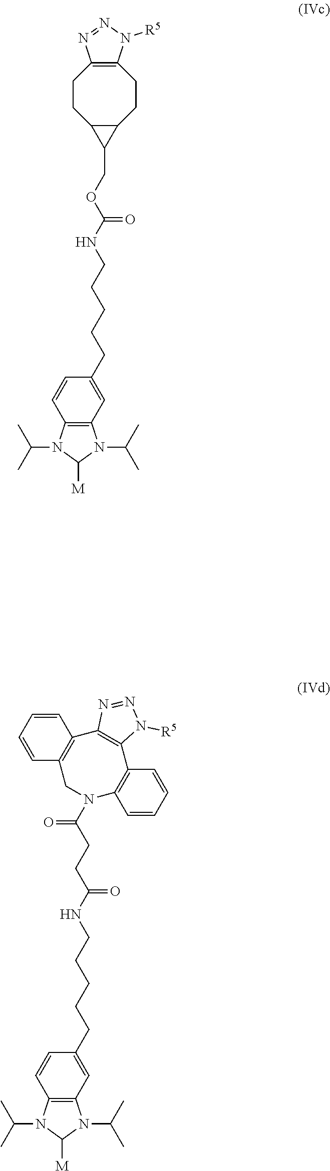

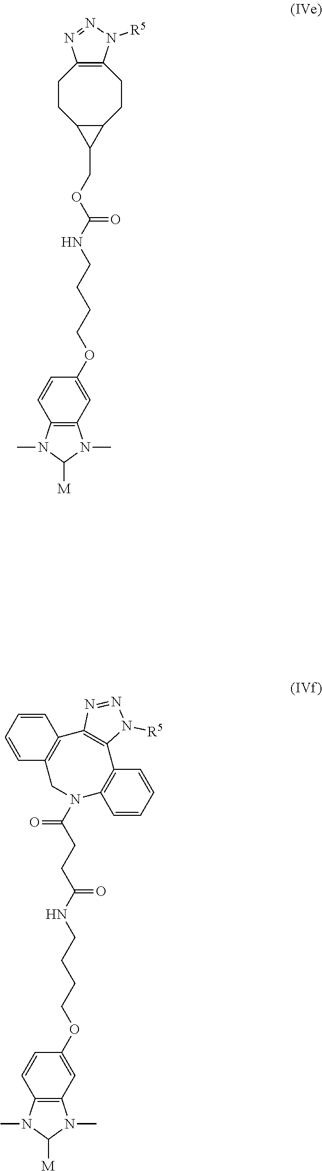

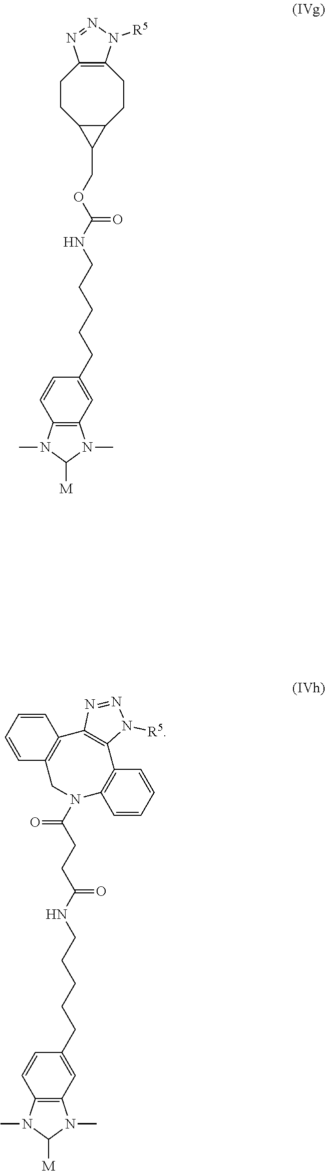

Although, the present disclosure has included many examples directed to the use of cyclooctyne-functionalized NHC compounds and compositions in the context E-AB type electrochemical biosensors, the present disclosure also contemplates their use in a wide variety of applications that utilize biomolecules specifically attached to metal surfaces other than electrodes, for example metal surfaces used in spectroscopic methods (e.g., SERS), or mass spectrometry (e.g., LDI-MS or MALDI-MS). As depicted in FIG. 4B, it is also contemplated that the cyclooctyne-functionalized NHCs of the present disclosure can be attached to the metal surfaces of nanoparticles and undergo SPAAC reaction to attach an aptamers. Accordingly, it is contemplated that the NHC compounds of the present disclosure can be used in applications that employ biomolecule-modified nanoparticles including but not limited to drug delivery applications.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Preparation of Exemplary Boc-Protected Dialkyl Benzimidazolium Precursor Compounds

This example illustrates the synthesis of exemplary Boc-protected dialkyl benzimidazolium compounds 3 and 4 are used as precursor compounds in the synthesis of N-heterocyclic carbene compounds of the present disclosure. Briefly, these precursor compounds 3 and 4 are prepared according to the synthesis scheme summarized in Scheme 1.

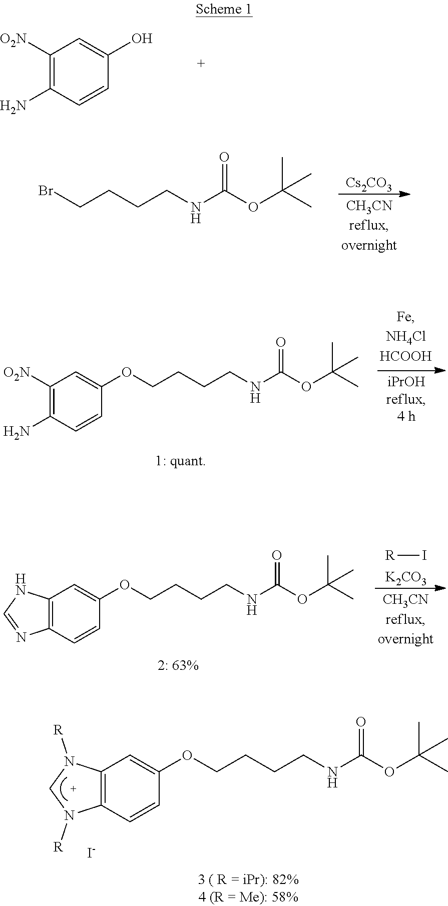

### A. Synthesis of Boc-protected 4-(4-aminobutoxy)-2-nitroaniline (compound 1)

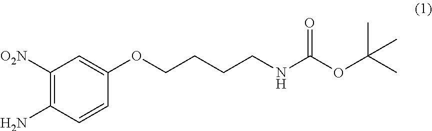 [SMILES: CC(C)(C)OC(=O)NCCCCOc1ccc(N)c([N+](=O)[O-])c1]

### Materials and Methods

The reaction is adapted from the previously reported procedure of ether synthesis. 4 4-amino-3-nitrophenol (1.68 g, 10.9 mmol, 1.0 eq.), tert-butyl (4-bromobutyl)carbamate (3.00 g, 11.9 mmol, 1.1 eq.), and cesium carbonate (20.0 g, 61.4 mmol, 5.5 eq.) were mixed in acetonitrile (50 mL). After the reaction was heated at reflux overnight under nitrogen atmosphere, the reaction was cooled down to room temperature. The reaction mixture was filtered through Celite pad and washed with acetonitrile (ca. 50 mL). The filtrate was condensed to obtain a crude product, which was further purified by flash column chromatography (50% v/v EtOAc in hexanes) to obtain an orange-red wax-like solid as a desired product.

### Results

Yield: 3.78 g, 11.7 mmol, quant.

¹H NMR (500 MHz, DMSO-d6): δ 7.35 (d, J=2.9 Hz, 1H, Ar H), 7.25 (br s, 2H, Ar NH2), 7.14 (dd, J=9.3, 2.9 Hz, 1H, Ar H), 6.99 (d, J=9.3 Hz, 1H, Ar H), 6.83 (t, J=5.7 Hz, 1H, —(C═O)NH—), 3.90 (t, J=6.4 Hz, 2H, —OCH2-), 2.96 (q, J=6.6 Hz, 2H, —(C═O)NHCH2-), 1.71-1.58 (m, 2H, —OCH2CH2-), 1.51 (q, J=7.5 Hz, 2H, —NHCH2CH2-), 1.37 (s, 9H, —C(CH3)3).

13C{1H}NMR (126 MHz, DMSO-d6): δ 155.6, 148.4, 141.9, 129.0, 127.5, 120.7, 105.8, 77.3, 67.8, 28.2, 28.2, 26.1, 26.0.

IR (ATR, neat): 3485, 3373, 3353, 2949, 2930, 2866, 1691, 1646, 1575, 1529, 1513, 1479, 1466, 1454, 1435, 1421, 1386, 1363, 1327, 1282, 1261, 1248, 1210, 1167, 1129, 1102, 1090, 1062, 1039, 996, 976, 945, 882, 855, 843, 799, 773, 761, 683, 611, 578.

HRMS (DART) m/z: calcd. 226.1187 for [M−Boc+2H]+=C10H16N3O3+, found 226.1156.

### B. Synthesis of Boc-protected 5-(4-aminobutoxy)-1H-benzo[d]imidazole (Compound 2)

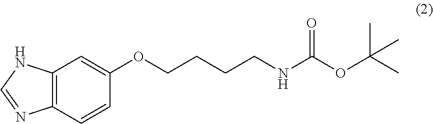 [SMILES: CC(C)(C)OC(=O)NCCCCOc1ccc2nc[nH]c2c1]

### Materials and Methods

The reaction was adapted from the previously reported procedure of benzimidazole synthesis. 4 Compound 1 (3.67 g, 11.3 mmol, 1.0 eq.), iron powder (9.00 g, 161 mmol, 14.3 eq.), and ammonium chloride (22.0 g, 411 mmol, 27.5 eq.) were suspended in 2-propanol (30 mL) and degassed with N2 for 10 minutes. Then, formic acid (15 mL) was added at room temperature and heated at reflux for 4 hours. The reaction was then cooled down to room temperature and filtered through a Celite pad on the glass frit to remove the undissolved residues. The filtrate from this step was concentrated using a rotary evaporator, and a saturated sodium bicarbonate solution (ca. 30 mL) was slowly added to remove remaining formic acid. This step was followed by extraction with EtOAc (20 mL×3 times) and the solution was dried over anhydrous sodium sulfate, filtered, and dry under vacuum to obtain the pale-yellow sticky solid as the desired product.

### Results

Yield: 2.16 g, 7.07 mmol, 63%

1H NMR (500 MHz, CDCl3): δ 8.01 (s, 1H, Ar H), 7.53 (d, J=8.8 Hz, 1H, Ar H), 7.04 (s, 1H, Ar H), 6.90 (dd, J=8.8, 2.3 Hz, 1H, Ar H), 4.71 (s, 1H, (C═O)NH), 3.97 (t, J=6.2 Hz, 2H, —OCH2-), 3.20 (q, J=6.7 Hz, 2H, —(C═O)NHCH2-), 1.83-1.79 (m, 2H, —OCH2CH2-), 1.72-1.64 (m, 2H, —NHCH2CH2-), 1.45 (s, 9H, —C(CH3)3).

13C{1H}NMR (126 MHz, CDCl3): δ 156.3, 156.1, 140.2, 137.4, 133.0, 116.8, 113.4, 98.5, 79.1, 68.3, 28.7, 28.6, 27.0, 26.7.

IR (ATR, neat): 3206, 2974, 2932, 2871, 2817, 1681, 1629, 1592, 1512, 1489, 1476, 1454, 1391, 1365, 1350, 1299, 1279, 1251, 1203, 1160, 1109, 1063, 1038, 1014, 989, 946, 810, 779, 761, 645, 622, 584, 571, 555.

HRMS (DART) m/z: calcd. 306.1813 for [M+H]+=C16H24N3O3+, found 306.1810.

### C. Synthesis of Boc-protected 5-(4-aminobutoxy)-1,3-diisopropyl-1H-benzo[d]imidazol-3-ium iodide (Compound 3)

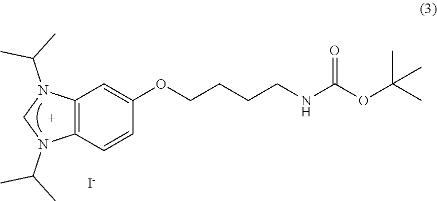 [SMILES: CC(C)N1CN(C(C)C)c2cc(OCCCCNC(=O)OC(C)(C)C)ccc21 ; [I-]]

### Materials and Methods

5-(4-(N-Boc-amino)butoxy)-1H-benzo[d]imidazole (2) (2.00 g, 6.56 mmol, 1.0 eq.), 2-iodopropane (6.00 g, 37.8 mmol, 5.8 eq.), and potassium carbonate (6.80 g, 49.2 mmol, 7.5 eq.) were mixed in acetonitrile (20 mL), then refluxed overnight. After the reaction was cooled down, water (ca. 50 mL) was added to the reaction mixture, followed by extraction with dichloromethane (50 mL×3 times). The organic layer was separated, dried over anhydrous magnesium sulfate, and concentrated using a rotovap. After the organic residue was concentrated, diethyl ether was added to crash out a white solid. The product was filtered, washed with diethyl ether, and dried under vacuum, which obtained a white solid powder as a desired product.

### Results

Yield: 2.79 g, 5.39 mmol, 82%

1H NMR (500 MHz, DMSO-d6): δ 9.63 (s, 1H, Ar H), 8.00 (d, J=9.2 Hz, 1H, Ar H), 7.60 (d, J=2.3 Hz, 1H, Ar H), 7.27 (dd, J=9.1, 2.3 Hz, 1H, Ar H), 6.85 (d, J=6.1 Hz, 1H, NH), 5.17-4.86 (m, 2H, —CH(CH3)2), 4.12 (t, J=6.5 Hz, 2H, —OCH2-), 2.99 (q, J=6.7 Hz, 2H, —NHCH2-), 1.75 (p, J=6.8 Hz, 2H, —OCH2CH2-), 1.66-1.59 (m, 12H, —CH(CH3)2), 1.55 (p, J=7.2 Hz, 2H, —NHCH2CH2-), 1.37 (s, 9H, —C(CH3)3).

13C{1H}NMR (126 MHz, DMSO-d6): δ 157.9, 155.7, 137.9, 131.9, 124.7, 116.7, 114.8, 97.0, 77.4, 68.5, 54.9, 50.8, 50.2, 28.3, 26.1, 25.9, 21.6.

IR (ATR, neat): 3302, 3117, 2975, 2933, 2874, 1691, 1623, 1552, 1500, 1464, 1433, 1391, 1364, 1314, 1237, 1204, 1166, 1139, 1109, 1079, 1067, 1018, 986, 925, 864, 817, 781, 744, 709, 696, 671, 640, 624, 614, 607, 601, 595, 584, 577, 564, 557.

HRMS (ESI) m/z: calcd. 390.2752 for [M−I+H]+=C22H36N3O3+, found 390.2748.

### D. Synthesis of Boc-protected 5-(4-aminobutoxy)-1,3-dimethyl-1H-benzo[d]imidazol-3-ium iodide (Compound 4)

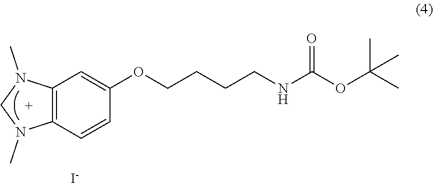 [SMILES: CN1CN(C)c2cc(OCCCCNC(=O)OC(C)(C)C)ccc21 ; [I-]]

### Materials and Methods

5-(4-(N-Boc-amino)butoxy)-1H-benzo[d]imidazole (2) (1.00 g, 3.28 mmol, 1.0 eq.), 2-iodomethane (2.78 g, 19.6 mmol, 6 eq.), and potassium carbonate (4.53 g, 32.8 mmol, 10.0 eq.) were mixed in acetonitrile (20 mL), and refluxed overnight. The synthetic procedure followed the synthesis of 3, obtaining off-white solid as a desired product.

### Results

Yield: 870 mg, 1.89 mmol, 58%

1H NMR (500 MHz, DMSO-d6): δ 9.50 (s, 1H, Ar H), 7.88 (d, J=9.1 Hz, 1H, Ar H), 7.58-7.49 (m, 1H, Ar H), 7.34-7.20 (m, 1H, Ar H), 6.84 (t, J=5.8 Hz, 1H, —(COO)NH—), 4.14-4.08 (m, 2H, —OCH2-), 4.04 (s, 3H, NCH3), 4.02 (s, 3H, NCH3), 2.99 (q, J=6.7 Hz, 2H, —NHCH2-), 1.76 (q, J=7.4 Hz, 2H, —OCH2CH2-), 1.61-1.47 (m, 2H, —NHCH2CH2-), 1.37 (s, 9H, —C(CH3)3).

13C{1H}NMR (126 MHz, DMSO-d6): δ 157.9, 155.6, 142.2, 132.8, 125.9, 116.6, 114.2, 96.5, 77.4, 68.4, 33.2, 33.1, 28.2, 28.2, 26.1, 25.9.

IR (ATR, neat): 3506, 3437, 3308, 3153, 3003, 2976, 2872, 1705, 1631, 1578, 1520, 1507, 1464, 1435, 1407, 1388, 1363, 1344, 1319, 1277, 1238, 1221, 1171, 1155, 1131, 1103, 1041, 1014, 1000, 987, 927, 873, 852, 809, 783, 746, 721, 695, 633, 590, 578, 572, 564, 556.

HRMS (DART) m/z: calcd. 320.1975 for [M−1-CH3+H]+=C17H26N3O3+, found 320.1969.

### Example 2: Preparation of Exemplary Cyclooctyne-Functionalized N-Heterocyclic Carbene (NHC) Isopropyl “Wingtip” Compounds 5, 6, 7, and 8

This example illustrates the synthesis of exemplary cyclooctyne-functionalized N-heterocyclic carbene (NHC) of the present disclosure. Briefly, precursor compound 3, prepared as described in Example 1, is used to prepare the BCN cyclooctyne-functionalized N-heterocyclic carbene (NHC) iodide compounds 5 and 6, and then the DBCO cyclooctyne-functionalized N-heterocyclic carbene (NHC) bicarbonate compounds 7 and 8, as summarized in Scheme 2A (see above).

### A. Synthesis of BCN Diisopropyl Benzimidazolium Iodide Compound 5

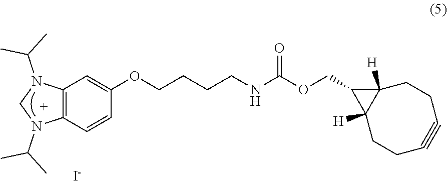 [SMILES: CC(C)N1CN(C(C)C)c2cc(OCCCCNC(=O)OC[C@@H]3[C@@H]4CCC#CCC[C@@H]43)ccc21 ; [I-]]

### Materials and Methods

In a 20-mL vial, compound 3 (340 mg, 0.657 mmol, 1.0 eq.) was dissolved in a mixture of trifluoroacetic acid (6 mL) and dichloromethane (6 mL). After the reaction stirred at room temperature for 1 h, the solvent and acid in the crude reaction were removed under vacuum to obtain a deprotected amino-tailed NHC as a sticky dark yellow solid (intermediate S1, see below).

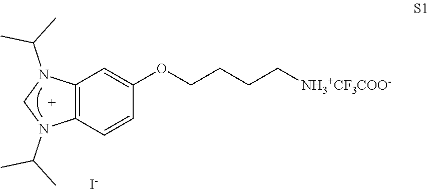

S1 was analyzed from the crude solid and confirmed to be the expected intermediate compound based on ¹H and ¹³C NMR. The S1 intermediate solid was directly used and redissolved in a mixture of Et₃N (4 mL) and dichloromethane (10 mL), followed by addition of BCN—NHS (200 mg, 0.687 mmol, 1.05 eq.). After the reaction stirred at room temperature overnight, the solvent in the reaction mixture was removed under vacuum, followed by adding deionized water (ca. 10 mL), and extracting with dichloromethane (ca. 10 mL×3 times). The organic layer was separated, dried over anhydrous magnesium sulfate, concentrated, and triturate in diethyl ether (ca. 10 mL×2 times). The sticky yellow liquid found on this step was dried under the vacuum line for 2 hours to obtain compound 5 as a highly hygroscopic yellow powder as a desired product.

### Results

Yield: 185 mg, 0.312 mmol, 48%

1H NMR (500 MHz, DMSO-d6): δ 9.65 (s, 1H, Ar H), 8.01 (d, J=9.2 Hz, 1H, Ar H), 7.61 (d, J=2.3 Hz, 1H, Ar H), 7.27 (dd, J=9.2, 2.3 Hz, 1H, Ar H), 7.16 (t, J=5.8 Hz, 1H, —O(C═O)NH—), 5.09-4.97 (mult, —CH(CH3)2), 4.12 (t, J=6.5 Hz, 2H, —OCH2CH2-), 4.03 (d, J=8.0 Hz, 2H, —OCH2CH—), 3.05 (q, J=6.6 Hz, 2H, —CH2CH2NH—), 2.22 (dd, J=15.7, 11.7 Hz, 2H, C≡C— CH2BCNV), 2.14 (td, J=10.9, 6.0 Hz, 4H, CH2BCN), 1.81-1.72 (m, 2H, CH2BCN), 1.62 (d, J=6.7 Hz, —CH(CH3)2), 1.59-1.54 (m, 2H, CH2BCN), 1.54-1.44 (m, 2H, CH2BCN), 1.27 (p, J=8.4 Hz, 1H, —CH2CH(CH-)2BCN), 0.90-0.78 (m, 2H, —CH(CH-)2BCN).

13C{1H}NMR (126 MHz, DMSO-d6): δ 157.8, 156.4, 137.9, 131.9, 124.7, 116.7, 114.8, 99.0, 97.0, 68.4, 61.2, 50.7, 50.2, 28.6, 26.1, 25.8, 21.5, 20.8, 19.5, 17.6.

IR (neat): 3286, 3116, 2978, 2937, 2851, 1684, 1624, 1554, 1501, 1468, 1437, 1404, 1377, 1241, 1197, 1165, 1114, 1066, 1025, 988, 937, 911, 894, 820, 799, 771, 733, 717, 644, 621, 599, 584, 566.

HRMS (LDI) m/z: calcd. 466.3065 for [M−I]+=C28H40N3O3+, found 466.3062.

### B. Synthesis of BCN Diisopropyl Benzimidazolium Bicarbonate Compound 6

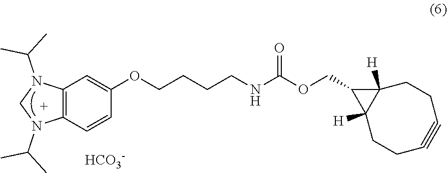

### Materials and Methods

Bicarbonate-exchange resin (HCO₃-resin) was prepared by the protocol that previously reported Amberlyst™ A-26(OH) resin (10 g) was suspended and stirred in ultra-high purity water (20 mL) while bubbling with CO₂ gas for 1 hour at room temperature. Then, 5 mL of resin was filtered and washed with methanol (10 mL×3 times). Benzimidazolium iodide 5 (185 mg, 0.312 mmol) was dissolved in methanol (5 mL), then added with freshly prepared HCO₃-resin. After the reaction was stirred at room temperature for 1 hour, the solution was filtered out from HCO₃-resin and washed with methanol (5 mL×3 times), and dried under vacuum, to obtain yellow powder solid as a desired product.

### Results

Yield: 110 mg, 0.208 mmol, 67%

1H NMR (500 MHz, DMSO-d6): δ 9.93 (s, 1H, Ar H), 8.01 (d, J=9.2 Hz, 1H, Ar H), 7.61 (d, J=2.3 Hz, 1H, Ar H), 7.26 (dd, J=9.1, 2.2 Hz, 1H, Ar H), 7.20 (t, J=5.7 Hz, 1H, NH), 5.09-4.97 (m, 2H, —CH(CH3)2), 4.12 (t, J=6.5 Hz, 2H, —OCH2CH2-), 4.03 (d, J=8.0 Hz, 2H, —(C═O)OCH2CH—), 3.05 (q, J=6.6 Hz, 2H, —CH2CH2NH—), 2.22 (t, J=13.9 Hz, 2H, —C≡C—CH2BCN), 2.14 (tt, J=11.9, 6.1 Hz, 4H, CH2BCN), 1.80-1.73 (m, 2H, CH2BCV), 1.65-1.54 (m, 14H, —CH(CH3)2 and CH2BCN), 1.54-1.48 (m, 2H, CH2BCN), 1.26 (p, J=8.2 Hz, 1H, —CH2CH(CH-)2BCN), 0.85 (dd, J=13.2, 6.4 Hz, 2H, —CH(CH-)2BCN).

13C{1H}NMR (126 MHz, DMSO-d6): δ 157.8, 157.6, 156.5, 138.6, 131.9, 124.7, 116.6, 114.8, 99.0, 97.0, 68.4, 61.2, 50.8, 50.7, 50.2, 28.6, 26.1, 25.9, 21.5, 20.8, 19.5, 17.7.

IR (ATR, neat): 3270, 3117, 2976, 2934, 2659, 1696, 1620, 1553, 1501, 1467, 1436, 1376, 1240, 1212, 1171, 1138, 1112, 1067, 1024, 987, 910, 894, 835, 766, 733, 687, 644, 593, 580, 563, 554,

HRMS (ESI) m/z: calcd. 466.3065 for [M−I]+=C28H40N3O3+, found 466.3086.

### C. Synthesis of DBCO Diisopropyl Benzimidazolium Iodide Compound 7

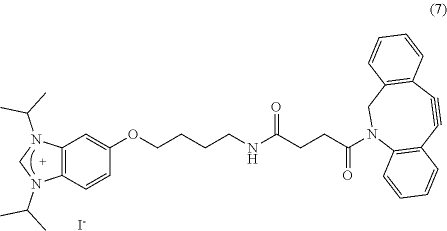 [SMILES: CC(C)N1CN(C(C)C)c2cc(OCCCCNC(=O)CCC(=O)N3Cc4ccccc4C#Cc4ccccc43)ccc21 ; [I-]]

### Materials and Methods

In a 20-mL vial, compound 3 (306 mg, 0.592 mmol, 1.0 eq.) was dissolved in a mixture of CF₃COOH (3 mL) and dichloromethane (5 mL) and followed the same synthetic method of compound 5, excepted changing BCN—NHS to DBCO—NHS (250 mg, 0.621 mmol, 1.05 eq.) with using 4 mL of triethylamine and 8 mL dichloromethane. An off-white solid was obtained after dried under the vacuum line as a desired product.

### Results

Yield: 380 mg, 91%

1H NMR (500 MHz, DMSO-d6): δ 9.63 (s, 1H, Ar HBzIm), 8.00 (d, J=9.2 Hz, 1H, Ar HBzIm), 7.72 (t, J=5.7 Hz, 1H, (C═O)NH), 7.69-7.65 (m, 1H, Ar HDBCO), 7.61 (dd, J=7.2, 5.5 Hz, 1H, Ar HDBCO), 7.59 (d, J=2.3 Hz, 1H, Ar HBzIm), 7.53-7.42 (m, 4H, Ar HDBCO), 7.35 (dtd, J=15.4, 7.4, 1.7 Hz, 3H, Ar HDBCO), 7.29 (dd, J=7.1, 1.8 Hz, 1H, Ar HDBCO), 7.25 (dd, J=9.1, 2.3 Hz, 1H, Ar HBzIm), 5.05-4.96 (m, 3H, —CH(CH3)2 and CHHDBCO), 4.08 (t, J=6.5 Hz, 2H, —OCH2-), 3.61 (d, J=14.0 Hz, 1H, CHHDBCO), 3.01 (q, J=6.6 Hz, 2H, —NHCH2-), 2.62-2.54 (m, 1H, —(C═O)CHH—), 2.23 (dt, J=15.3, 7.7 Hz, 1H, —(C═O)CHH—), 2.00 (ddd, J=14.7, 8.0, 5.8 Hz, 1H, —(C═O)CHH—), 1.73-1.65 (m, 2H, —(C═O)CHH—), 1.64-1.57 (m, 12H, —CH(CH3)2), 1.48 (p, J=7.2 Hz, 2H, —NHCH2CH2-).

13C{1H}NMR (126 MHz, DMSO-d6): δ 171.0, 170.9, 157.8, 151.6, 148.4, 137.9, 132.4, 131.9, 129.6, 128.9, 128.1, 127.9, 127.6, 126.8, 125.1, 124.7, 122.5, 121.4, 116.7, 114.8, 114.2, 108.1, 96.9, 68.3, 54.9, 50.7, 50.2, 38.0, 30.4, 29.7, 25.9, 25.6, 21.5.

IR (ATR, neat): 3284, 2982, 2936, 2161, 1688, 1652, 1554, 1501, 1480, 1467, 1432, 1396, 1324, 1284, 1239, 1198, 1168, 1121, 1035, 1008, 823, 799, 770, 754, 717, 643, 570, 561.

HRMS (ESI) m/z: calcd. 577.3174 for [M−I]+=C36H41N4O3+, found 577.3185.

### D. Synthesis of DBCO Diisopropyl Benzimidazolium Bicarbonate Compound 8

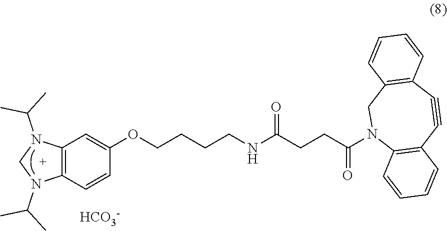

### Materials and Methods

Benzimidazolium iodide 7 (340 mg, 0.483 mmol) was dissolved in methanol (7 mL), added with freshly prepared HCO3-resin (7 mL) and followed the same synthetic method of compound 6. After the reaction was stirred at room temperature for 2 hours, the solution was filtered out from HCO3-resin and washed with methanol (5 mL×3 times), and dried under vacuum, to obtain off-white solid powder as a desired product.

### Results

Yield: 140 mg, 45%

1H NMR (500 MHz, DMSO-d6): δ 9.81 (s, 1H, Ar HBzIm), 8.00 (d, J=9.2 Hz, 1H, Ar HBzIm), 7.80 (t, J=5.7 Hz, 1H, (C═O)NH), 7.68 (dd, J=7.1, 1.8 Hz, 1H, Ar HDBCO), 7.63-7.57 (m, 2H, Ar HDBCO and Ar HBzIm), 7.52-7.43 (m, 3H, Ar HDBCO), 7.40-7.32 (m, 2H, Ar HDBCO), 7.29 (dd, J=7.3, 1.9 Hz, 1H, Ar HDBCO), 7.25 (dd, J=9.2, 2.3 Hz, 1H, Ar HBzIm), 5.06-4.97 (m, 3H, —CH(CH3)2 and CHHDBCO), 4.08 (t, J=6.5 Hz, 2H, —OCH2-), 3.61 (d, J=14.0 Hz, 2H, CHHDBCO), 3.01 (q, J=6.7 Hz, 2H, —NHCH2-), 2.58 (dt, J=15.9, 7.7 Hz, 1H, —N(C═O)CHH—), 2.23 (dt, J=15.3, 7.6 Hz, 1H, —NH(C═O)CHH—), 2.01 (ddd, J=14.7, 8.1, 5.8 Hz, 1H, —NH(C═O)CHH—), 1.82-1.75 (m, 1H, —N(C═O)CHH—), 1.70 (p, J=6.6 Hz, 2H, —OCH2CH2-), 1.61 (t, J=6.4 Hz, 12H, —CH(CH3)2), 1.48 (p, J=7.4 Hz, 2H, —NHCH2CH2-).

13C{1H}NMR (126 MHz, DMSO-d6): δ 171.1, 170.9, 157.8, 155.6, 151.6, 148.4, 138.3, 132.4, 131.9, 129.6, 128.9, 128.1, 127.9, 127.6, 126.8, 125.1, 124.7, 122.5, 121.4, 116.6, 114.8, 114.2, 108.1, 97.0, 68.3, 54.9, 50.7, 50.2, 38.0, 30.4, 29.7, 25.9, 25.6, 21.55, 21.53.

IR (ATR, neat): 3271, 3060, 2975, 2935, 2874, 2161, 1641, 1554, 1501, 1480, 1466, 1433, 1377, 1349, 1288, 1240, 1209, 1169, 1138, 1111, 1074, 1035, 1007, 988, 954, 882, 860, 824, 769, 754, 729, 717, 688, 644, 612, 579, 570, 563, 554.

HRMS (ESI) m/z: calcd. 577.3174 for [M−HCO3-]+=C36H41N4O3+, found 577.3189.

### Example 3: Preparation of Exemplary Cyclooctyne-Functionalized N-Heterocyclic Carbene (NHC) Methyl “Wingtip” Compounds 9 and 10

This example illustrates the synthesis of exemplary cyclooctyne-functionalized N-heterocyclic carbene (NHC) of the present disclosure. Briefly, the precursor compound 4 is used to prepare the DBCO cyclooctyne-functionalized N-heterocyclic carbene (NHC) compounds 9 or 10 according to the synthesis scheme summarized in Scheme 2B (see above).

### A. Synthesis of DBCO Dimethyl Benzimidazolium Iodide Compound 9

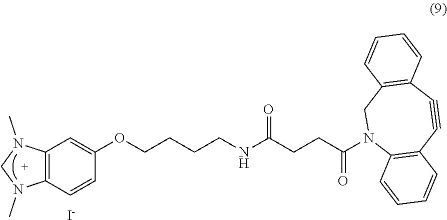 [SMILES: CN1CN(C)c2cc(OCCCCNC(=O)CCC(=O)N3Cc4ccccc4C#Cc4ccccc43)ccc21 ; [I-]]

### Materials and Methods

In a 20-mL vial, compound 4 (200 mg, 0.434 mmol, 1.0 eq.) was dissolved in a mixture of CF₃COOH (2 mL) and dichloromethane (4 mL) and followed the same synthetic method of compound 7 using DBCO—NHS (183 mg, 0.456 mmol, 1.05 eq.) with triethylamine (4 mL) and dichloromethane (4 mL). An off-white solid was obtained after dried under the vacuum line as a desired product.

### Results

Yield: 110 mg, 39%

1H NMR (500 MHz, DMSO-d6): δ 9.66 (s, 1H, Ar HBzIm), 7.84 (d, J=9.1 Hz, 1H, Ar HBzIm), 7.72 (t, J=5.7 Hz, 1H, (C═O)NH), 7.67 (dd, J=7.2, 1.8 Hz, 1H, Ar HDBCO), 7.61 (dd, J=7.3, 1.7 Hz, 1H, Ar HDBCO), 7.51-7.42 (m, 4H, Ar HDBCO and Ar HBzIm), 7.38-7.30 (m, 2H, Ar HDBCO), 7.28 (dd, J=7.2, 1.8 Hz, 1H, Ar HDBCO), 7.23 (dd, J=9.1, 2.2 Hz, 1H, Ar HBzIm), 5.02 (d, J=14.1 Hz, 1H, CHHDBCO), 4.12-3.98 (m, 8H, —CH3 and —OCH2-), 3.61 (d, J=14.0 Hz, 1H, CHHDBCO), 3.01 (q, J=6.6 Hz, 2H, —NHCH2-), 2.58 (dt, J=15.9, 7.7 Hz, 1H, —N(C═O)CHH—), 2.23 (dt, J=15.3, 7.6 Hz, 1H, —NH(C═O)CHH—), 2.00 (ddd, J=15.3, 8.0, 5.8 Hz, 1H, —NH(C═O)CHH—), 1.78 (ddd, J=16.4, 7.9, 5.8 Hz, 1H, —N(C═O)CHH—), 1.69 (p, J=6.8 Hz, 2H, —OCH2CH2-), 1.47 (p, J=7.3 Hz, 2H, —NHCH2CH2-).

13C{1H}(126 MHz, DMSO-d6): δ 171.0, 170.9, 157.8, 151.6, 148.4, 143.0, 132.8, 132.4, 129.6, 128.9, 128.1, 127.9, 127.6, 126.7, 125.9, 125.1, 122.5, 121.4, 116.3, 114.2, 114.1, 108.1, 96.4, 68.3, 54.9, 38.0, 33.2, 33.0, 30.4, 29.7, 25.9, 25.6.

IR (ATR, neat): 3276, 3066, 2934, 2163, 1648, 1574, 1508, 1481, 1467, 1448, 1398, 1350, 1320, 1273, 1246, 1200, 1103, 1009, 821, 754, 718, 608, 559.

### B. Synthesis of DBCO Dimethyl Benzimidazolium Carboxylate Compound 10

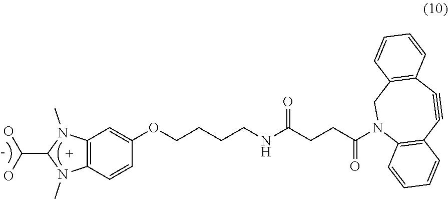 [SMILES: CN1c2ccc(OCCCCNC(=O)CCC(=O)N3Cc4ccccc4C#Cc4ccccc43)cc2N(C)C1C(O)O]

### Materials and Methods

DBCO dimethyl benzimidazolium iodide salt 9 (90 mg, 0.139 mmol) was dissolved in methanol (5 mL), added with freshly prepared HCO₃-resin (5 mL) and followed the same synthetic method of compound 6. After the reaction was stirred at room temperature for 2 hours, the solution was filtered out from HCO₃-resin and washed with methanol (5 mL×3 times), and dried under vacuum, to obtain pale yellow solid powder as a desired product.

### Results

Yield: 46 mg, 57%

1H NMR (500 MHz, DMSO-d6) δ 7.81 (d, J=9.0 Hz, 1H, Ar HBzIm), 7.75 (t, J=5.6 Hz, 1H, (C═O)NH), 7.67 (dd, J=7.2, 1.7 Hz, 1H, Ar HDBCO), 7.61 (dd, J=7.2, 1.7 Hz, 1H, Ar HDBCO), 7.52-7.42 (m, 4H, Ar HDBCO and Ar HBzIm), 7.39-7.30 (m, 2H, Ar HDBCO), 7.28 (dd, J=7.3, 1.8 Hz, 1H, Ar HDBCO), 7.20 (dd, J=9.0, 2.3 Hz, 1H, Ar HBzIm), 5.02 (d, J=14.0 Hz, 1H, CHHDBCO), 4.16-3.84 (m, 8H, —CH3 and —OCH2-), 3.60 (d, J=14.0 Hz, 1H, CHHDBCO), 3.05-2.97 (m, 2H, —NHCH2-), 2.58 (dt, J=15.9, 7.7 Hz, 1H, —N(C═O)CHH—), 2.23 (dt, J=15.3, 7.6 Hz, 1H, —NH(C═O)CHH—), 2.01 (ddd, J=14.8, 8.0, 5.8 Hz, 1H, —NH(C═O)CHH—), 1.78 (ddd, J=16.3, 8.0, 5.8 Hz, 1H, —N(C═O)CHH—), 1.69 (p, J=6.7 Hz, 2H, —OCH2CH2-), 1.53-1.42 (m, 2H, —NHCH2CH2-).

13C{1H}NMR (126 MHz, DMSO-d6) δ 171.1, 170.9, 157.8, 153.9, 151.6, 148.4, 147.0, 132.4, 132.0, 129.6, 128.9, 128.1, 127.9, 127.6, 126.8, 125.1, 125.0, 122.5, 121.4, 116.4, 114.2, 114.1, 108.1, 96.4, 68.2, 54.9, 38.0, 32.6, 32.6, 30.4, 29.7, 26.0, 25.6.

IR (ATR, neat): 3281, 3060, 2930, 2161, 1631, 1573, 1508, 1480, 1466, 1433, 1385, 1348, 1273, 1244, 1154, 1101, 1007, 836, 770, 753, 729, 690, 631, 583, 557.

HRMS (DART) m/z: calcd. 521.2548 for [M−CO2]+=C32H33N4O3+, found 521.2565.

### Example 4: SPAAC Click Reactions with BCN Cyclooctyne-Functionalized N-Heterocyclic Carbene (NHC) Compound 6

This example illustrates the effectiveness of the BCN cyclooctyne-functionalized NHC compound 6 with azide compounds to form click products. Compound 6 was reacted with 1-hexylazide (C₆H₁₃N₃) and methylene blue azide (MB-N₃) to form the exemplary click product compounds 11 and 12. Briefly, compound 6 is reacted with 1-hexylazide (C₆H₁₃N3) or methylene blue azide (MB-N₃) according to the scheme summarized in Scheme 3A The reactions were performed in solution at room temperature and monitored by ¹H NMR. Additionally, ¹³C NMR and 2D NMR techniques were employed to assign resonances for compounds 11 and 12.

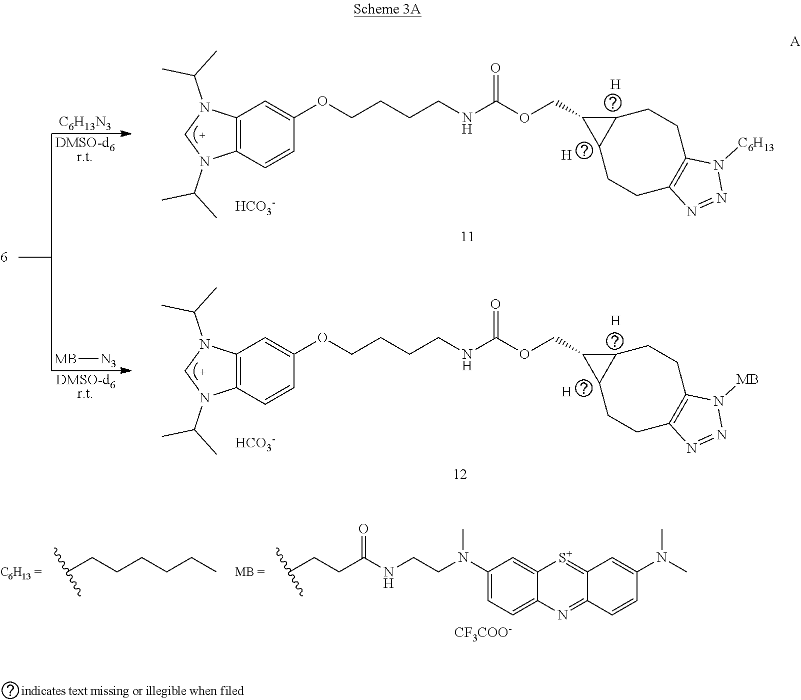

### A. Click Reaction of Compound 6 with Hexylazide to Form Compound 11

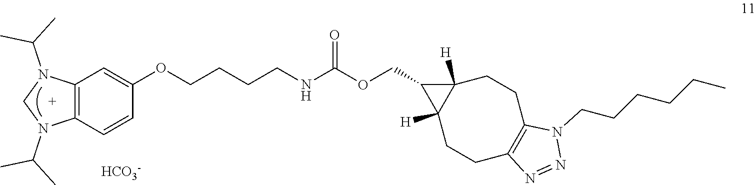

### Materials and Methods

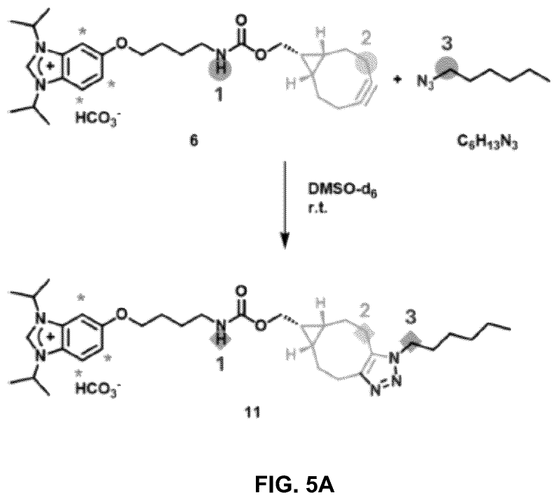

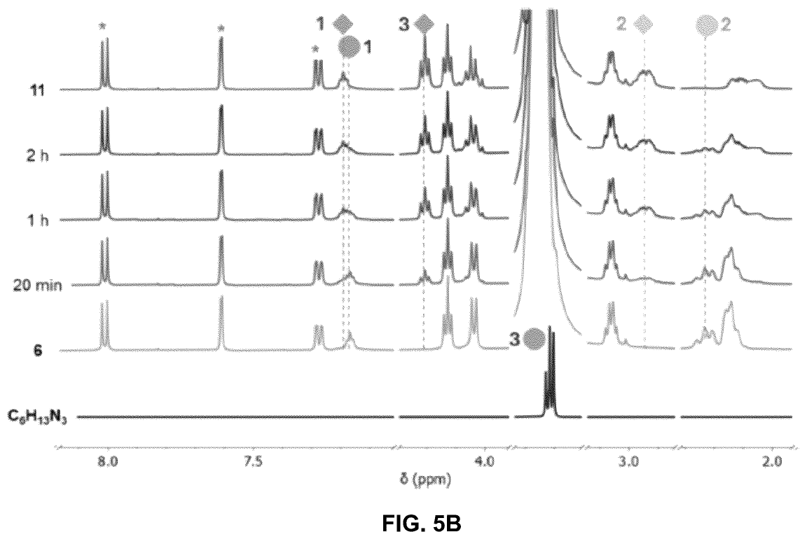

Compound 6 (10 mg, 19 μmol, 1 eq.) was dissolved in 500 μL DMSO-d6 in the NMR tube, followed by adding 1-hexylazide (9 mg, 76 μmol, 4 eq.). The reaction mixture was allowed to react at room temperature and monitored by 1H NMR until all initial signals of compound 6 were converted to compound 11, which was completed in 1 hour. The compound 6 proton signals between 2.0 and 2.3 ppm were assigned to the 6-position protons of CH on the BCN moiety disappeared with formation of compound 11 (see e.g., position labeled “2” in structure of FIG. 5A and corresponding NMR peaks labeled “2” in FIG. 5B). In addition, in the ¹³C NMR spectrum the disappearance of the compound 6 alkyne signal at 99.0 ppm and the emergence of the compound 11 alkene signals at 132.7 and 143.4 ppm confirming formation of the triazole ring.

Kinetics of the click reaction also were monitored via ¹H NMR using 3.7 mM of compound 6 in DMSO-d6 with 1 equiv of C₆H₁₃N₃ at room temperature. Compound 6 (1.0 mg, 1.9 μmol) was dissolved in DMSO-d6 (500 μL) in the NMR tube, then 1-hexylazide (0.24 mg, 1.9 μmol) in 20 μL DMSO-d6 was added. The reaction was reacted at room temperature and monitored by 1H NMR spectroscopy The plot between 1/[compound 6](M⁻¹) versus time t (s) (not shown) was fitted with a linear equation and calculated the slope (m) for the k value (M-1·s-1) using the integral value of 1.0 mg of compound 6 in 520 μL DMSO-d6 as an external standard at t=0. The reaction was mostly completed after 9 h of the reaction time and fully converted after 18 h, and demonstrated the expected second order kinetics with a rate constant, k of 0.072 M⁻¹·s⁻¹.

### Results

1H NMR (500 MHz, DMSO-d6): δ 9.70 (s, 1H, Ar H), 8.00 (d, J=9.2 Hz, 1H, Ar H), 7.60 (d, J=2.3 Hz, 1H, Ar H), 7.27 (dd, J=9.1, 2.3 Hz, 1H, Ar H), 7.18 (t, J=6.0 Hz, 1H, —NH(COO)—), 5.01 (dp, J=13.3, 6.7 Hz, 2H, —CH(CH3)2), 4.20 (t, J=7.2 Hz, 2H, —NCH2-), 4.12 (t, J=6.5 Hz, 2H, —OCH2-), 4.09-3.98 (m, 2H, —(C═O)OCH2-), 3.05 (q, J=6.8 Hz, 2H, —NHCH2-), 2.93 (ddd, J=15.9, 7.3, 3.6 Hz, 2H, CH2BCN), 2.70 (dddd, J=26.7, 15.3, 10.8, 5.0 Hz, 2H, CH2BCN), 2.16-1.98 (m, 2H, CH2BCN), 1.77 (p, J=6.8 Hz, 2H, —CH2-), 1.68 (q, J=6.9 Hz, 2H, —CH2-), 1.65-1.59 (m, 12H, —CH(CH3)2), 1.00-0.89 (m, 2H, —CH2-).

13C{1H}NMR (126 MHz, DMSO-d6): δ 157.9, 156.8, 156.5, 143.4, 138.1, 132.7, 131.9, 124.7, 116.7, 114.8, 97.0, 68.4, 61.3, 54.9, 50.7, 50.2, 48.6, 47.1, 40.4, 30.6, 29.5, 26.1, 25.9, 25.6, 25.3, 22.1, 22.0, 21.9, 21.6, 21.4, 19.2, 18.7, 17.4.

HRMS (ESI) m/z: calcd. 593.4174 for [M−HCO3]+=C34H53N6O3+, found 593.4166.

### B. Click Reaction of Compound 6 with Methylene Blue Azide to Form Compound 12

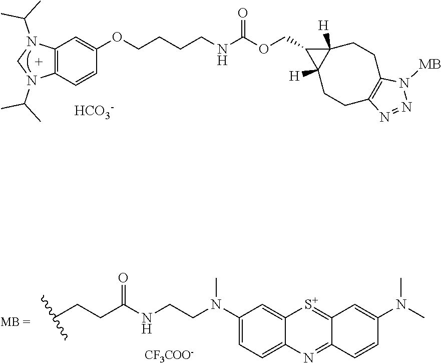

### Materials and Methods

Compound 6 was reacted with MB-N₃ to yield target compound 12 under the same conditions as for the reaction of compound 6 with hexylazide (above). Kinetics of the reaction of compound 6 with MB-N₃ were measured by dissolving MB-N₃ (1.0 mg, 1.9 μmol) in DMSO-d6 (500 μL) in an NMR tube, and adding compound 6 (1.0 mg, 1.9 μmol) in 20 μL DMSO-d6 into the tube. The reaction proceeded at room temperature and was monitored by 1H NMR spectroscopy. The decrease in concentration of compound 6 was calculated from the triplet peak at 2.2 ppm and fitted as a linear equation, where 1.0 mg of compound 6 in 520 μL of DMSO-d6 was used as an external standard at t=0. The kinetics for the formation of compound 12 showed the expected second-order reaction with a rate constant of 0.047 M⁻¹s⁻¹.

### Results

HRMS (ESI) m/z: calcd. 989.4678 for [M−HCO₃]+=C₅₀H₆₄F₃N₁₀O₆S⁺, found 989.4661.

### Example 5: SPAAC Click Reactions with DBCO Cyclooctyne-Functionalized N-Heterocyclic Carbene (NHC) Compound 8

This example illustrates the effectiveness of the DBCO cyclooctyne-functionalized NHC compound 8 with azide compounds to form click products. Compound 8 was reacted with 1-hexylazide (C₆H₁₃N₃) and methylene blue azide (MB-N₃) to form the exemplary click product compounds 13 and 14. Briefly, compound 8 is reacted with 1-hexylazide (C₆H₁₃N₃) or methylene blue azide (MB-N₃) according to the scheme summarized in Scheme 3AB The reactions were performed in solution at room temperature and monitored by ¹H NMR. Additionally, ¹³C NMR and 2D NMR techniques were employed to assign resonances for compounds 13 and 14.

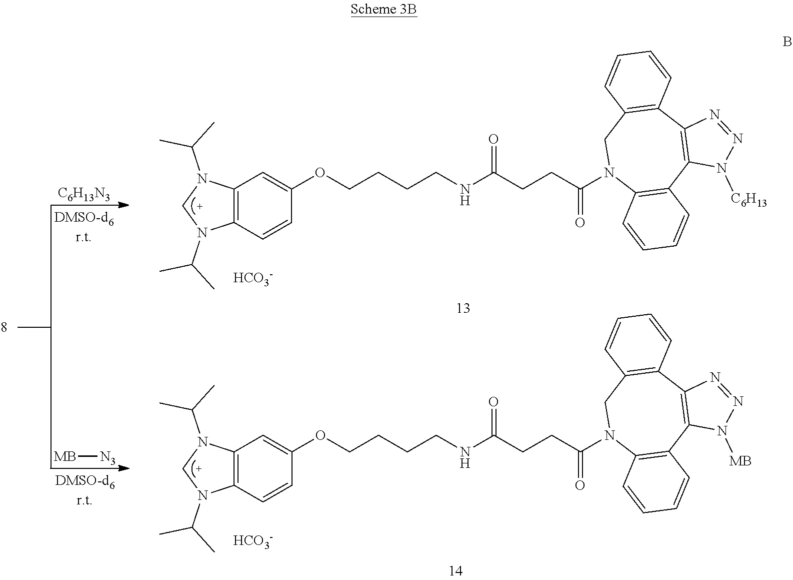

### A. Click Reaction of Compound 8 with Hexylazide to Form Compound 13

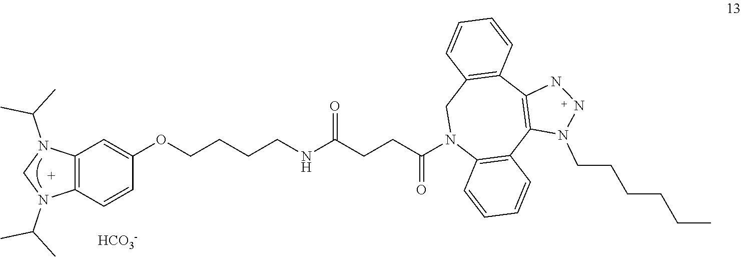

### Materials and Methods

Compound 8 (10 mg, 16 μmol, 1 eq.) was dissolved in 500 μL DMSO-d6 in the NMR tube, followed by adding 1-hexylazide (8 mg, 64 μmol, 4 eq.). The reaction mixture was allowed to react at room temperature and monitored by ¹H NMR until all initial signals of compound 8 were converted to compound 13 in 15 minutes. Two isomers of compound 13 were identified in the ¹H NMR spectrum with ca. mole ratio of 1:1.

The 1H and 13C NMR results of 13 confirmed the formation of clicked product. An appearance of two doublet 1H signal at 5.93 and 5.83 ppm represented a methylene proton of DBCO of 13, while the other proton was found in the region of 4.45-4.41 ppm by 1H-1H COSY. The amide proton of 13 was also clearly identified with two triplet signals of amide at 7.86 and 7.79 ppm that were split due to asymmetry of two isomers. Furthermore, 13C NMR spectrum comparison between 8 and 13 showed three important findings which included: (1) the disappearance of alkyne (108.1 and 114.2 ppm) and quaternary carbon signals (114.8, 116.7, 148.4, and 151.6 ppm) of 8's DBCO due to the click reaction, (2) increasing the amount of 13C signals in an aromatic region (120-145 ppm) due to the two isomers formation, and (3) amide peak splitting at 170 ppm, confirmed the formation of 1,2,3-triazole moiety. Meanwhile, the 13C signals of benzimidazole unit (96.9, 114.8, 116.6, 124.7, 131.9, 138.3, and 157.8 ppm) were located on the same chemical shift compared to 8 as it was separate from DBCO unit beyond 12 bonds.

Kinetics of the reaction were measured by dissolving compound 8 (1.2 mg, 1.9 μmol) in DMSO-d6 (500 μL) in an NMR tube, followed by addition of 1-hexylazide (0.24 mg, 1.9 μmol) in 20 μL DMSO-d6. The reaction proceeded at room temperature and was monitored by 1H NMR spectroscopy. The decrease in the concentration of compound 8 was calculated from the doublet peak at 5.1 ppm and fitted as a linear equation, where 1.2 mg of compound 8 in 520 μL of DMSO-d6 was used as an external standard at t=0. The expected second order reaction kinetics were observed with a rate constant, k=0.63 M⁻¹s⁻¹.

### Results

1H NMR (500 MHz, DMSO-d6): δ 9.83 (s, 1H, Ar HBzIm), 8.00 (dd, J=9.2, 1.8 Hz, 1H, Ar HBzIm), 7.86 (t, J=5.7 Hz, % H, NH), 7.79 (t, J=5.6 Hz, % H, NH), 7.70-7.63 (m, 1H, Ar HDBCO), 7.63-7.54 (m, 3H, Ar HDBCO and Ar HBzIm), 7.54-7.45 (m, 1H, Ar HDBCO), 7.45-7.40 (m, 1H, Ar HDBCO), 7.40-7.37 (m, 1H, Ar HDBCO), 7.37-7.30 (m, % H, Ar HDBCO), 7.30-7.22 (m, 3H, Ar HDBCO and Ar HBzIm), 5.93 (d, J=16.9 Hz, % H, CHHDBCO), 5.83 (d, J=17.1 Hz, % H, CHHDBCO), 5.10-4.95 (m, 2H, —CH(CH3)2), 4.55-4.41 (m, 2H, (1H) CHHDBCO), 4.41-4.32 (m, 1H), 4.27 (t, J=7.6 Hz, 1H), 4.16-4.02 (m, 2H, —OCH2-), 3.38 (q, J=7.0 Hz, 1H), 3.31 (t, J=6.9 Hz, 2H), 3.03 (p, J=6.3 Hz, 2H), 2.24-1.87 (m, 5H), 1.72 (p, J=7.1 Hz, 2H), 1.66-1.57 (m, 13H, —CH(CH3)2), 1.56-1.46 (m, 5H), 1.44-1.22 (m, 10H), 1.22-1.13 (m, 2H), 1.13-1.06 (m, 3H), 1.04 (d, J=6.3 Hz, 1H), 0.90-0.81 (m, 5H), 0.78 (t, J=7.1 Hz, 2H).

13C{1H}NMR (126 MHz, DMSO-d6): δ 170.7, 170.5, 170.2, 157.8, 143.8, 142.3, 141.1, 140.2, 138.3, 135.5, 133.9, 133.8, 132.1, 131.9, 131.8, 131.7, 131.2, 130.8, 130.6, 129.8, 129.7, 129.4, 129.2, 129.1, 128.7, 128.5, 128.4, 128.2, 127.4, 127.0, 126.8, 124.7, 124.4, 116.6, 114.8, 97.0, 96.9, 68.4, 64.9, 51.9, 50.8, 50.7, 50.5, 50.2, 48.3, 48.0, 40.4, 38.1, 38.0, 30.6, 30.4, 30.1, 30.0, 29.1, 29.0, 29.0, 26.0, 25.9, 25.7, 25.7, 25.1, 21.9, 21.7, 21.5, 15.1.

HRMS (ESI) m/z: calcd. 704.4283 for [M−HCO3]+=C42H54N7O3+, found 704.4272.

### B. Click Reaction of Compound 8 with Hexylazide to Form Compound 14

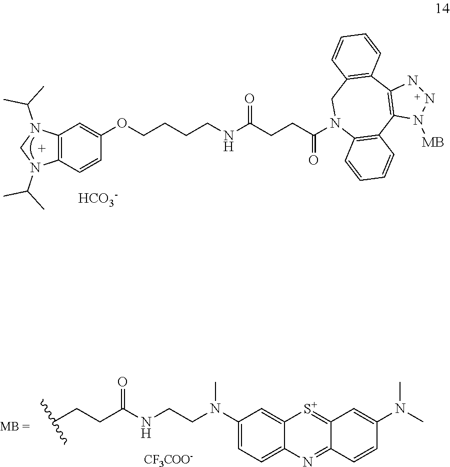

### Materials and Methods

Compound 8 was reacted with MB-N₃ to yield target compound 14 under the same conditions as for the reaction of compound 8 with hexylazide (above). Kinetics of the reaction of compound 8 with MB-N₃ were measured by dissolving MB-N₃ (1.0 mg, 1.9 μmol) was dissolved in DMSO-d6 (500 μL) in the NMR tube, and adding compound 8 (1.2 mg, 1.9 μmol) in 20 μL DMSO-d6. The reaction proceeded at room temperature and was monitored by 1H NMR spectroscopy. The decrease in concentration of compound 8 was calculated from the doublet peak at 5.1 ppm and fitted as a linear equation, where 1.2 mg of compound 8 in 520 μL of DMSO-d6 was used as an external standard at t=0. The kinetics for the formation of compound 14 showed the expected second-order reaction with a rate constant of 0.55 M⁻¹s⁻¹.

### Results

HRMS (ESI) m/z: calcd. 493.7466 for [M−HCO3-CF3COO]2+=C56H65N11O4S2+, found 493.7458.

### Example 6: Preparation of Exemplary Cyclooctyne-Functionalized N-Heterocyclic Carbene (NHC) Adduct on a Gold Electrode Surface and its Use as a Sensor

This example demonstrates the preparation of an exemplary N-heterocyclic carbene metal adduct of the present disclosure, the ability to attach an aptamer to this metal adduct in a SPAAC click reaction, and the use of the aptamer attached to the metal adduct as a sensor. Briefly, the DBCO dimethyl benzimidazolium carboxylate NHC of compound 10 was co-deposited on a gold surface along with a methyl-winged benzimidazolium NHC compound 15 using standard vacuum deposition techniques. Compound 15, which is not a cyclooctyne-functionalized NHC and accordingly is not modified with a target aptamer, acts as a blocking compound that provides sufficient spacing on the gold surface between the deposited cyclooctene-functionalized NHC of compound 10, thereby provided sufficient spacing for effective aptamer-target interaction and biosensor signal transduction.

### A. Synthesis of NHC Blocking Compound: 1,3-dimethyl-1H-benzimidazol-3-ium-2-carboxylate (Compound 15)

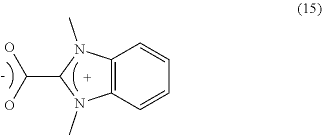

### Materials and Methods

1,3-dimethyl-1H-benzimidazolium iodide (500 mg, 1.82 mmol), prepared by the previously reported procedure. Compound 6 was dissolved in methanol (6 mL), followed by adding 8 mL of HCO₃-resin to the solution. After the reaction was stirred at room temperature for 2 hours, the resin was removed by filtration. The solution was dried under vacuum to obtain an off-white solid powder as a desired product compound 15. A bicarbonate form of compound 15, 1,3-dimethyl-1H-benzimidazol-3-ium bicarbonate (compound 15-HCO₃), was also formed as a minor product.

### Results

Yield: 145 mg, 0.76 mmol, 42% (0.77:0.23 of 15:15-HCO3 by mole, calculated by NMR integration)

1H NMR (500 MHz, DMSO-d6): δ 7.94 (dd, J=6.2, 3.1 Hz, 2H, Ar H), 7.64 (dd, J=6.2, 3.1 Hz, 2H, Ar H), 4.14 (s, 6H, —CH3).

13C{1H}NMR (126 MHz, DMSO-d6): δ 153.8 (—COO—), 147.7, 130.9, 126.3, 113.3, 32.6.

HRMS (DART) m/z: calcd. 175.0866 for [M−O+H]+=C10H11N2O+, found 175.0854.

### B. Co-Deposition of Compounds 10 and 15 on a Gold Surface

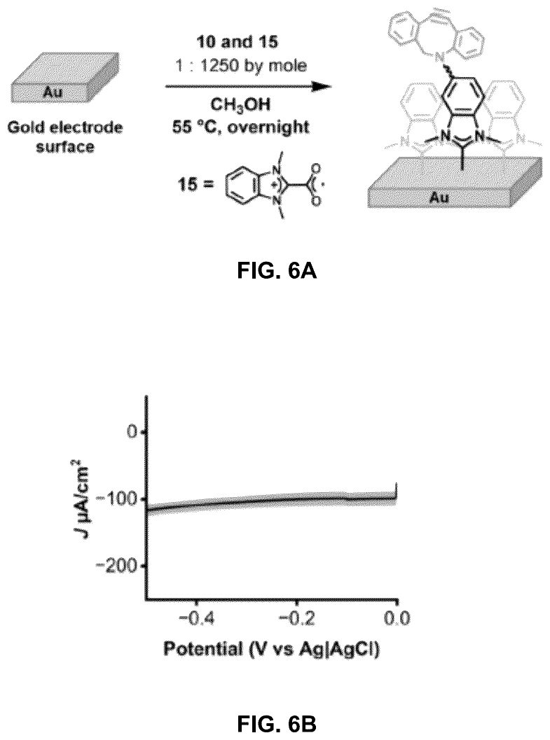

The cyclooctyne functionalized NHC compound 10 was co-deposited on the surface of a gold electrode along with the blocking NHC compound 15 using standard vacuum deposition techniques. A mole ratio of 1:1250 compound 10 to blocking compound 15 was identified empirically as resulting in voltammograms with the best balance between the lowest oxygen reduction currents and the largest Faradaic currents from the methylene blue. Accordingly, as depicted in the schematic of FIG. 6A, a methanolic solution mixture of compounds 10 and 15 at a 1:1250 mole ratio was deposited on the gold electrode at 55° C. overnight to form a layer of NHCs on gold electrode surfaces. The prepared electrode was interrogated via square wave voltammetry and a drop in the differential current density down to −100 μA/cm² relative to bare gold electrodes was observed (see FIG. 6B) confirming the deposition of the NHC compounds on the gold surface. Additionally, the shape of the voltammogram (as shown in FIG. 6B) reflects a flat capacitive current at a potential range where electrochemical reduction of dissolved molecular oxygen is known to occur (<−0.4 V vs Ag/AgCl), which is also a strong indication of successful electrode passivation.

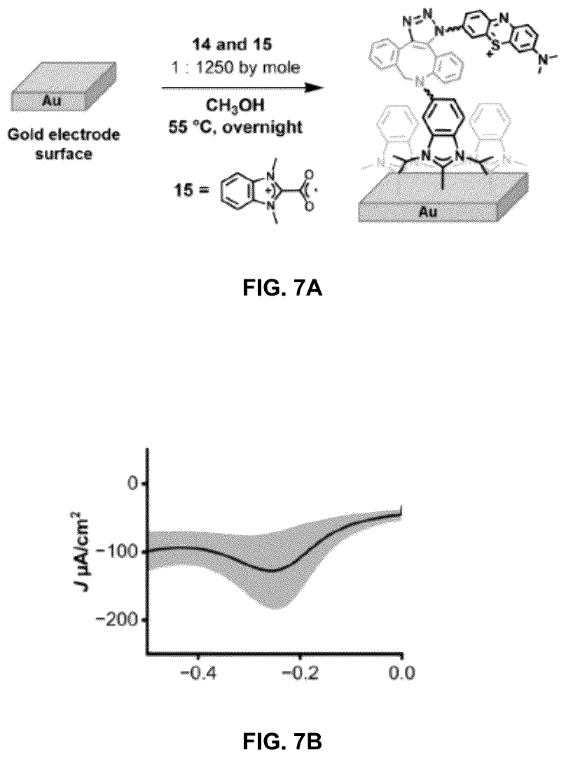

A positive control study as illustrated schematically in FIG. 7A was carried out. A methanolic solution mixture of the “pre-clicked” NHC of compound 14 and the blocking NHC of compound 15 at a 1:1250 mole ratio was deposited on the gold electrode at 55° C. overnight. The prepared positive control electrode was interrogated via square wave voltammetry and a redox wave was observed at a formal redox potential of ˜−0.24 V as shown in FIG. 7B. This redox potential is as expected for a methylene blue moiety attached to the pre-clicked NHC of compound 14.

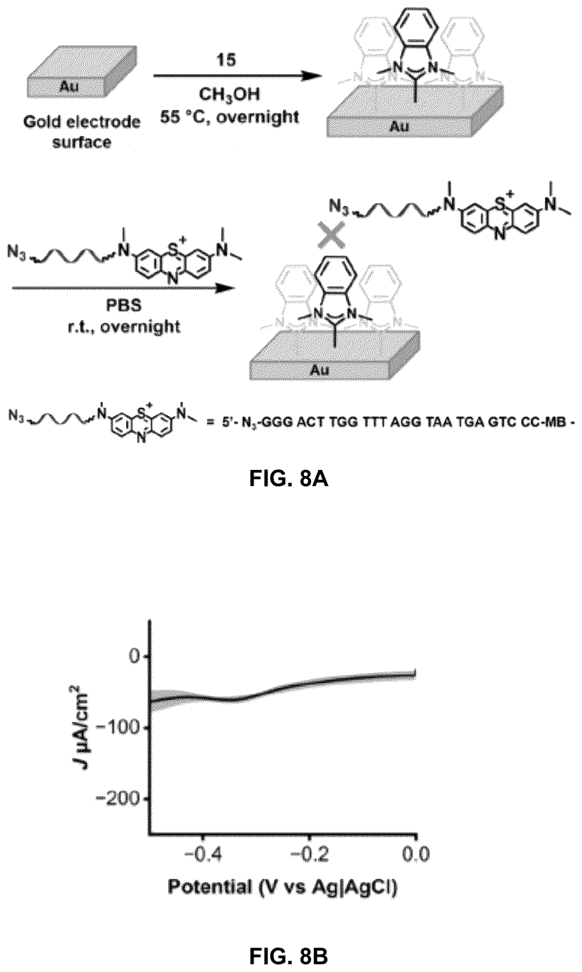

A negative control study as illustrated schematically in FIG. 8A was also carried out. In this study, only the blocking NHC of compound 15 was deposited on a gold electrode. A 500 nM solution of a modified DNA aptamer in phosphate-buffered saline was then deposited on the electrode surface overnight at room temperature. As depicted in FIG. 8A, the modified DNA aptamer is an oligonucleotide sequence 5′-GGGACTTCCTTTAGGTAATGAGTCCC-3′ modified with a 5′-azide (“N3”) group and a 3′-methylene blue (“MB”) group. As shown in FIG. 8B, square wave voltammograms of this negative control electrode produced no significant signal compared to the positive control, indicating that there was limited nonspecific adsorption of the aptamer.

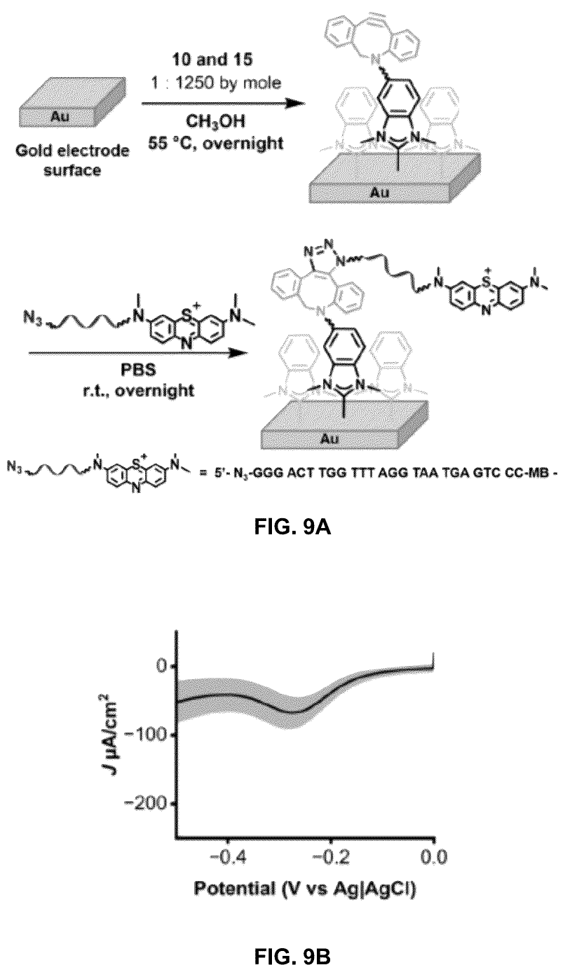

Finally, a study was carried out as depicted in FIG. 9A. A gold electrode surface prepared with a co-deposited mixed monolayer with cyclooctyne functionalized NHC compound 10 and blocking compound 15 in 1:1250 mole ratio. A 500 nM solution of the modified DNA aptamer in phosphate-buffered saline was then deposited on this prepared electrode surface overnight at room temperature. Following the overnight treatment with the modified DNA aptamer, square wave voltammetry of the electrode surface revealed an electrochemical process at ˜−0.25 V (see FIG. 9B) indicative of a successful SPAAC click reaction to compound 10 resulting in a methylene blue moiety attached. In addition, the redox currents appeared at potentials less negative than those observed for the no-DBCO control experiment and were 3-fold higher in magnitude, confirming the specificity of the SPAAC click reaction.

### REFERENCES

(1) Saha, K.; Agasti, S. S.; Kim, C.; Li, X.; Rotello, V. M. Gold Nanoparticles in Chemical and Biological Sensing. Chem. Rev. 2012, 112 (5), 2739-2779.
(2) Yi, J.; Xianyu, Y. Gold Nanomaterials-Implemented Wearable Sensors for Healthcare Applications. Adv. Funct. Mater. 2022, 32 (19), 2113012.
(3) Zamani, M.; Klapperich, C. M.; Furst, A. L. Recent advances in gold electrode fabrication for low-resource setting biosensing. Lab Chip 2023, 23 (5), 1410-1419.
(4) Wu, Y.; Arroyo-Curras, N. Advances in nucleic acid architectures for electrochemical sensing. Curr. Opin. Electrochem. 2021, 27, 100695.
(5) Wu, Y.; Ranallo, S.; Del Grosso, E.; Chamoro-Garcia, A.; Ennis, H. L.; Milosavic, N.; Yang, K.; Kippin, T.; Ricci, F.; Stojanovic, M.; Plaxco, K. W. Using Spectroscopy to Guide the Adaptation of Aptamers into Electrochemical Aptamer-Based Sensors. Bioconjugate Chem. 2023, 34 (1), 124-132.
(6) Parolo, C.; Greenwood, A. S.; Ogden, N. E.; Kang, D.; Hawes, C.; Ortega, G.; Arroyo-Curras, N.; Plaxco, K. W. E-DNA scaffold sensors and the reagentless, single-step, measurement of HIVdiagnostic antibodies in human serum. Microsyst. Nanoeng. 2020, 6(1), 13.
(7) Shaver, A.; Mahlum, J. D.; Scida, K.; Johnston, M. L.; Aller-Pellitero, M.; Wu, Y.; Carr, G. V.; Arroyo-Curras, N. Optimization of Vancomycin Aptamer Sequence Length Increases the Sensitivity of Electrochemical, Aptamer-Based Sensors In Vivo. ACS Sens. 2022, 7(12), 3895-3905.
(8) Tsai, Y.-C.; Weng, W.-Y.; Yeh, Y.-T.; Chien, J.-C. Dual-Aptamer Drift Canceling Techniques to Improve Long-Term Stability of Real-Time Structure-Switching Aptasensors. ACS Sens. 2023, 8 (9), 3380-3388.
(9) Li, H.; Dauphin-Ducharme, P.; Arroyo-Curras, N.; Tran, C. H.; Vieira, P. A.; Li, S.; Shin, C.; Somerson, J.; Kippin, T. E.; Plaxco, K. W. A Biomimetic Phosphatidylcholine-Terminated Monolayer Greatly Improves the In Vivo Performance of Electrochemical Aptamer-Based Sensors. Angew. Chem., Int. Ed. 2017, 56 (26), 7492-7495.
(10) Li, S.; Dai, J.; Zhu, M.; Arroyo-Curras, N.; Li, H.; Wang, Y.; Wang, Q.; Lou, X.; Kippin, T. E.; Wang, S.; Plaxco, K. W.; Li, H.; Xia, F. Implantable Hydrogel-Protective DNA Aptamer-Based Sensor Supports Accurate, Continuous Electrochemical Analysis of Drugs at Multiple Sites in Living Rats. ACS Nano 2023, 17 (18), 18525-18538.
(11) Downs, A. M.; Gerson, J.; Hossain, M. N.; Ploense, K.; Pham, M.; Kraatz, H.-B.; Kippin, T.; Plaxco, K. W. Nanoporous Gold for the Miniaturization of In Vivo Electrochemical Aptamer-Based Sensors. ACS Sens. 2021, 6 (6), 2299-2306.
(12) Reynoso, M.; Chang, A.-Y.; Wu, Y.; Murray, R.; Suresh, S.; Dugas, Y.; Wang, J.; Arroyo-Curras, N. 3D-printed, aptamer-based microneedle sensor arrays using magnetic placement on live rats for pharmacokinetic measurements in interstitial fluid. Biosens. Bioelectron. 2024, 244, 115802.
(13) Scida, K.; Ornelas-Gatdula, E.; De Pasquale, M.; Carr, G. V.; Arroyo-Curras, N. Therapeutic Drug Distribution across the Mouse Brain Is Heterogeneous as Revealed by In Vivo, Spatially Resolved Aptamer-Based Sensing. ACS Pharmacol. Transl. Sci. 2025, 8 (2), 435-445.
(14) McConnell, E. M.; Nguyen, J.; Li, Y. Aptamer-Based Biosensors for Environmental Monitoring. Front. Chem. 2020, 8, 434.
(15) Yu, H.; Alkhamis, O.; Canoura, J.; Liu, Y.; Xiao, Y. Advances and Challenges in Small-Molecule DNA Aptamer Isolation, Characterization, and Sensor Development. Angew. Chem., Int. Ed. 2021, 60 (31), 16800-16823.
(16) Li, J.; Clark, V.; Yu, C.-H.; Scida, K.; Aller Pellitero, M.; Rivera, R. L. A.; Zhong, W.; Demek, E.; Fountain, J.; Mahlum, J. D.; et al. Monitoring HIV Antiretroviral Therapy via Aptamer-Based Measurements in Preclinical Animal Models, in Human Plasma. Adv. Sens. Res. 2025, 4, 2400191.
(17) Lee, H.; Xie, T.; Kang, B.; Yu, X.; Schaffter, S. W.; Schulman, R. Plug-and-play protein biosensors using aptamer-regulated in vitro transcription. Nat. Commun. 2024, 15 (1), 7973.
(18) Clark, V.; Waters, K.; Orsburn, B.; Bumpus, N. N.; Kundu, N.; Sczepanski, J. T.; Ray, P.; Arroyo-Curras, N. Human Cyclophilin B Nuclease Activity Revealed via Nucleic Acid-Based Electrochemical Sensors. Angew. Chem., Int. Ed. 2022, 61 (45), No. e202211292.
(19) Amini, R.; Zhang, Z.; Li, J.; Gu, J.; Brennan, J. D.; Li, Y. Aptamers for SARS-CoV-2: Isolation, Characterization, and Diagnostic and Therapeutic Developments. Anal. Sens. 2022, 2 (5), No. e202200012.
(20) Fontaine, N.; Dauphin-Ducharme, P. Confounding effects on the response of electrochemical aptamer-based biosensors. Curr. Opin. Electrochem. 2023, 41, 101361.
(21) Arroyo-Curras, N.; Dauphin-Ducharme, P.; Ortega, G.; Ploense, K. L.; Kippin, T. E.; Plaxco, K. W. Subsecond-Resolved Molecular Measurements in the Living Body Using Chronoamperometrically Interrogated Aptamer-Based Sensors. ACS Sens. 2018, 3 (2), 360-366.
(22) Santos-Cancel, M.; Lazenby, R. A.; White, R. J. Rapid Two-Millisecond Interrogation of Electrochemical, Aptamer-Based Sensor Response Using Intermittent Pulse Amperometry. ACS Sens. 2018, 3(6), 1203-1209.
(23) Shaver, A.; Arroyo-Curras, N. Expanding the monolayer scope for nucleic acid-based electrochemical sensors beyond thiols on gold: Alkylphosphonic acids on ITO. ECS Sens. Plus. 2023, 2 (1), 010601.
(24) Shaver, A.; Curtis, S. D.; Arroyo-Curras, N. Alkanethiol Monolayer End Groups Affect the Long-Term Operational Stability and Signaling of Electrochemical, Aptamer-Based Sensors in Biological Fluids. ACS Appl. Mater. Interfaces 2020, 12 (9), 11214-11223.
(25) Bakestani, R. M.; Wu, Y.; Glahn-Martinez, B.; Kippin, T. E.; Plaxco, K. W.; Kolkman, R. W. Carboxylate-Terminated Electrode Surfaces Improve the Performance of Electrochemical Aptamer-Based Sensors. ACS Appl. Mater. Interfaces 2025, 17 (5), 8706-8714.
(26) Roehrich, B.; Leung, K. K.; Gerson, J.; Kippin, T. E.; Plaxco, K. W.; Sepunaru, L. Calibration-Free, Seconds-Resolved In Vivo Molecular Measurements using Fourier-Transform Impedance Spectroscopy Interrogation of Electrochemical Aptamer Sensors. ACS Sens. 2023, 8 (8), 3051-3059.
(27) Schoenfisch, M. H.; Pemberton, J. E. Air Stability of Alkanethiol Self-Assembled Monolayers on Silver and Gold Surfaces. J. Am. Chem. Soc. 1998, 120 (18), 4502-4513.
(28) Willey, T. M.; Vance, A. L.; van Buuren, T.; Bostedt, C.; Terminello, L. J.; Fadley, C. S. Rapid degradation of alkanethiol-based self-assembled monolayers on gold in ambient laboratory conditions. Surf. Sci. 2005, 576 (1), 188-196.
(29) Mani, G.; Johnson, D. M.; Marton, D.; Dougherty, V. L.; Feldman, M. D.; Patel, D.; Ayon, A. A.; Agrawal, C. M. Stability of Self-Assembled Monolayers on Titanium and Gold. Langmuir 2008, 24 (13), 6774-6784.
(30) Garg, N.; Carrasquillo-Molina, E.; Lee, T. R. Self-Assembled Monolayers Composed of Aromatic Thiols on Gold: Structural Characterization and Thermal Stability in Solution. Langmuir 2002, 18 (7), 2717-2726.
(31) Li, F.; Zhang, H.; Dever, B.; Li, X.-F.; Le, X. C. Thermal Stability of DNA Functionalized Gold Nanoparticles. Bioconjugate Chem. 2013, 24 (11), 1790-1797.
(32) Delamarche, E.; Michel, B.; Kang, H.; Gerber, C. Thermal Stability of Self-Assembled Monolayers. Langmuir 1994, 10 (11), 4103-4108.
(33) Flynn, N. T.; Tran, T. N. T.; Cima, M. J.; Langer, R. Long-Term Stability of Self-Assembled Monolayers in Biological Media. Langmuir 2003, 19 (26), 10909-10915.
(34) Maciel, J.; Martins, M. C. L.; Barbosa, M. A. The stability of self-assembled monolayers with time and under biological conditions. J. Biomed. Mater. Res., Part A 2010, 94A (3), 833-843.
(35) Leung, K. K.; Downs, A. M.; Ortega, G.; Kurnik, M.; Plaxco, K. W. Elucidating the Mechanisms Underlying the Signal Drift of Electrochemical Aptamer-Based Sensors in Whole Blood. ACS Sens. 2021, 6 (9), 3340-3347.
(36) Shaver, A.; Kundu, N.; Young, B. E.; Vieira, P. A.; Sczepanski, J. T.; Arroyo-Curras, N. Nuclease Hydrolysis Does Not Drive the Rapid Signaling Decay of DNA Aptamer-Based Electrochemical Sensors in Biological Fluids. Langmuir 2021, 37 (17), 5213-5221.
(37) Clark, V.; Pellitero, M. A.; Arroyo-Curras, N. Explaining the Decay of Nucleic Acid-Based Sensors under Continuous Voltammetric Interrogation. Anal. Chem. 2023, 95 (11), 4974-4983.
(38) Williams, K. P.; Liu, X.-H.; Schumacher, T. N. M.; Lin, H. Y.; Ausiello, D. A.; Kim, P. S.; Bartel, D. P. Bioactive and nuclease resistant L-DNA ligand of vasopressin. Proc. Natl. Acad. Sci. U.S.A 1997, 94 (21), 11285-11290.
(39) Young, B. E.; Kundu, N.; Sczepanski, J. T. Mirror-Image Oligonucleotides: History and Emerging Applications. Eur. J. Chem. 2019, 25 (34), 7981-7990.
(40) Chung, C.; Lee, M. Exchange of self-assembled thiol monolayers on gold: Characterization by FT-IR external reflection spectroscopy. J. Electroanal. Chem. 1999, 468 (1), 91-97.
(41) Schlenoff, J. B.; Li, M.; Ly, H. Stability and self-exchange in alkanethiol monolayers. J. Am. Chem. Soc. 1995, 117 (50), 12528-12536.
(42) Zhukhovitskiy, A. V.; MacLeod, M. J.; Johnson, J. A. Carbene Ligands in Surface Chemistry: From Stabilization of Discrete Elemental Allotropes to Modification of Nanoscale and Bulk Substrates. Chem. Rev. 2015, 115 (20), 11503-11532.
(43) Engel, S.; Fritz, E.-C.; Ravoo, B. J. New trends in the functionalization of metallic gold: From organosulfur ligands to Nheterocyclic carbenes. Chem. Soc. Rev. 2017, 46 (8), 2057-2075.
(44) Smith, C. A.; Narouz, M. R.; Lummis, P. A.; Singh, I.; Nazemi, A.; Li, C.-H.; Crudden, C. M. N-Heterocyclic Carbenes in Materials Chemistry. Chem. Rev. 2019, 119 (8), 4986-5056.
(45) Koy, M.; Bellotti, P.; Das, M.; Glorius, F. N-Heterocyclic carbenes as tunable ligands for catalytic metal surfaces. Nat. Catal. 2021, 4 (5), 352-363.
(46) Bellotti, P.; Koy, M.; Hopkinson, M. N.; Glorius, F. Recent advances in the chemistry and applications of N-heterocyclic carbenes. Nat. Rev. Chem. 2021, 5 (10), 711-725.
(47) Shen, H.; Tian, G.; Xu, Z.; Wang, L.; Wu, Q.; Zhang, Y.; Teo, B. K.; Zheng, N. N-heterocyclic carbene coordinated metal nanoparticles and nanoclusters. Coord. Chem. Rev. 2022, 458, 214425.
(48) Kaur, G.; Thimes, R. L.; Camden, J. P.; Jenkins, D. M. Fundamentals and applications of N-heterocyclic carbene functionalized gold surfaces and nanoparticles. Chem. Commun. 2022, 58 (95), 13188-13197.
(49) Mora, M.; Gimeno, M. C.; Visbal, R. Recent advances in gold-NHC complexes with biological properties. Chem. Soc. Rev. 2019, 48(2), 447-462.
(50) Lin, J. C. Y.; Huang, R. T. W.; Lee, C. S.; Bhattacharyya, A.; Hwang, W. S.; Lin, I. J. B. Coinage Metal-N-Heterocyclic Carbene Complexes. Chem. Rev. 2009, 109 (8), 3561-3598.
(51) Zhukhovitskiy, A. V.; Mavros, M. G.; Van Voorhis, T.; Johnson, J. A. Addressable Carbene Anchors for Gold Surfaces. J. Am. Chem. Soc. 2013, 135 (20), 7418-7421.
(52) Crudden, C. M.; Horton, J. H.; Ebralidze, I. I.; Zenkina, O. V.; McLean, A. B.; Drevniok, B.; She, Z.; Kraatz, H.-B.; Mosey, N. J.; Seki, T.; Keske, E. C.; Leake, J. D.; Rousina-Webb, A.; Wu, G. Ultra-stable self-assembled monolayers of N-heterocyclic carbenes on gold. Nat. Chem. 2014, 6 (5), 409-414.
(53) De Jesus, J. F.; Trujillo, M. J.; Camden, J. P.; Jenkins, D. M. N-Heterocyclic Carbenes as a Robust Platform for Surface-Enhanced Raman Spectroscopy. J. Am. Chem. Soc. 2018, 140 (4), 1247-1250.
(54) MacLeod, M. J.; Johnson, J. A. PEGylated N-Heterocyclic Carbene Anchors Designed To Stabilize Gold Nanoparticles in Biologically Relevant Media. J. Am. Chem. Soc. 2015, 137 (25), 7974-7977.
(55) Sherman, L. M.; Finley, M. D.; Borsari, R. K.; Schuster-Little, N.; Strausser, S. L.; Whelan, R. J.; Jenkins, D. M.; Camden, J. P. N-Heterocyclic Carbene Ligand Stability on Gold Nanoparticles in Biological Media. ACS Omega 2022, 7 (1), 1444-1451.
(56) Pellitero, M. A.; Jensen, I. M.; Dominique, N. L.; Ekowo, L. C.; Camden, J. P.; Jenkins, D. M.; Arroyo-Curras, N. Stability of N-Heterocyclic Carbene Monolayers under Continuous Voltammetric Interrogation. ACS Appl. Mater. Interfaces 2023, 15 (29), 35701-35709.
(57) Dominique, N. L.; Chandran, A.; Jensen, I. M.; Jenkins, D. M.; Camden, J. P. Unmasking the Electrochemical Stability of N-Heterocyclic Carbene Monolayers on Gold. Eur. J. Chem. 2024, 30(15), No. e202303681.
(58) Lee, D. S.; Zarabadi, M. P.; Bhattacharjee, H.; Qi, L.; McLeod, J. F.; Saeedfar, K.; Singh, I.; Woods, A.; Messina, A.; Birss, V. I.; Crudden, C. M.; She, Z. Toll like receptor-based electrochemical sensors via N-heterocyclic carbene-modified surfaces: Towards improved sensing of DNA molecules. Mater. Adv. 2024, 5 (15), 6063-6069.
(59) Kim, E.; Koo, H. Biomedical applications of copper-free click chemistry: In vitro, in vivo, and ex vivo. Chem. Sci. 2019, 10 (34), 7835-7851.
(60) Luu, T.; Gristwood, K.; Knight, J. C.; Jorg, M. Click Chemistry: Reaction Rates and Their Suitability for Biomedical Applications. Bioconjugate Chem. 2024, 35 (6), 715-731.
(61) Sletten, E. M.; Bertozzi, C. R. Bioorthogonal Chemistry: Fishing for Selectivity in a Sea of Functionality. Angew Chem., Int. Ed. 2009, 48 (38), 6974-6998.
(62) Nguyen, D. T.; Freitag, M.; Korsgen, M.; Lamping, S.; Ruhling, A.; Schafer, A. H.; Siekman, M. H.; Arlinghaus, H. F.; van der Wiel, W. G.; Glorius, F.; Ravoo, B. J. Versatile Micropatterns of N-Heterocyclic Carbenes on Gold Surfaces: Increased Thermal and Pattern Stability with Enhanced Conductivity. Angew. Chem., Int. Ed. 2018, 57 (35), 11465-11469.
(63) Eisen, C.; Keppler, B. K.; Chin, J. M.; Su, X.; Reithofer, M. R. Fabrication of azido-PEG-NHC stabilized gold nanoparticles as a functionalizable platform. Chem. Sci. 2024, 15 (44), 18524-18533.
(64) Tsvetkov, P.; Coy, S.; Petrova, B.; Dreishpoon, M.; Verma, A.; Abdusamad, M.; Rossen, J.; Joesch-Cohen, L.; Humeidi, R.; Spangler, R. D.; Eaton, J. K.; Frenkel, E.; Kocak, M.; Corsello, S. M.; Lutsenko, S.; Kanarek, N.; Santagata, S.; Golub, T. R. Copper induces cell death by targeting lipoylated TCA cycle proteins. Science 2022, 375 (6586), 1254-1261.
(65) Dommerholt, J.; van Rooijen, O.; Borrmann, A.; Guerra, C. F.; Bickelhaupt, F. M.; van Delft, F. L. Highly accelerated inverse electron-demand cycloaddition of electron-deficient azides with aliphatic cyclooctynes. Nat. Commun. 2014, 5 (1), 5378.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. An N-heterocyclic carbene compound of structural formula I 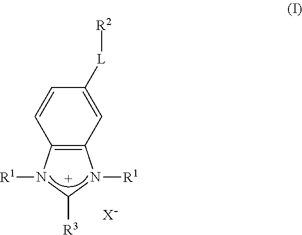
wherein,
R¹ is selected from —H, —CH₃, —CH₂—(CH₃)₂, —(C₁-C₆) linear or branched alkyl;
R² is a cyclooctyne group;
L is a linker comprising a covalently bonded chain of 2 to 100 atoms;
R³ is selected from —H and —COO⁻; and
X⁻ is an optional anion present when R³ is —H.

2. The compound of claim 1, wherein R¹ is —CH₃ or —CH₂—(CH₃)₂.

3. The compound of claim 1, wherein the linker L comprises one or more of the following chemical groups: linear (C₁-C₅) alkyl, linear (C₁-C₅) alkenyl, linear (C₁-C₅) alkynyl, ester, ether, amine, amide, imide, phosphodiester, and/or polyethylene glycol (PEG).

4. The compound of claim 1, L comprises a linker of structural formula L_{A}, L_{B}, or L_{C}

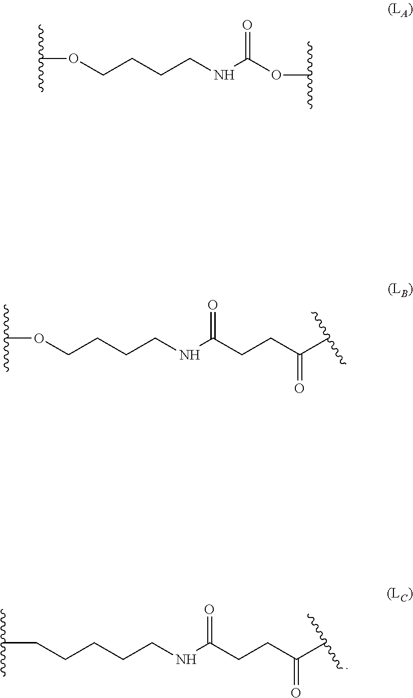 [SMILES: *OCCCCNC(=O)O* ; *OCCCCNC(=O)CCC(*)=O ; *CCCCCNC(=O)CCC(*)=O]

5. The compound of claim 1, wherein R² is a cyclooctyne group selected from IIa and IIb:

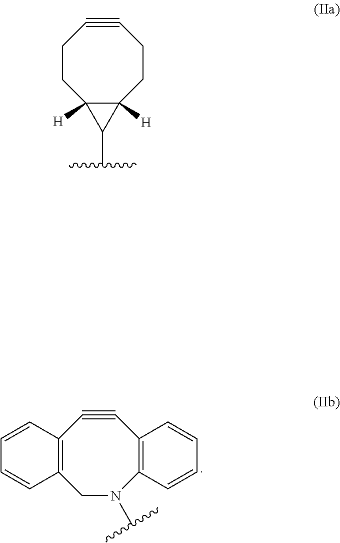 [SMILES: *C1[C@H]2CCC#CCC[C@@H]12 ; *N1Cc2ccccc2C#Cc2ccccc21]

6. The compound of claim 1, wherein R³ is —H and X⁻ is selected from HCO₃⁻ and a halide.

7. The compound of claim 1, wherein R³ is —COO⁻.

8. The compound of claim 1, wherein the compound of structural formula I is selected from compounds Ia, Ib, Ic, Id, Ie, If, Ig, and Ih:

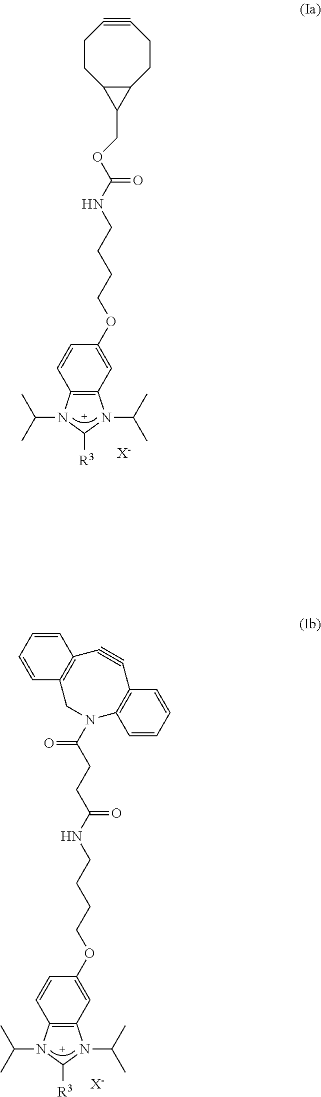

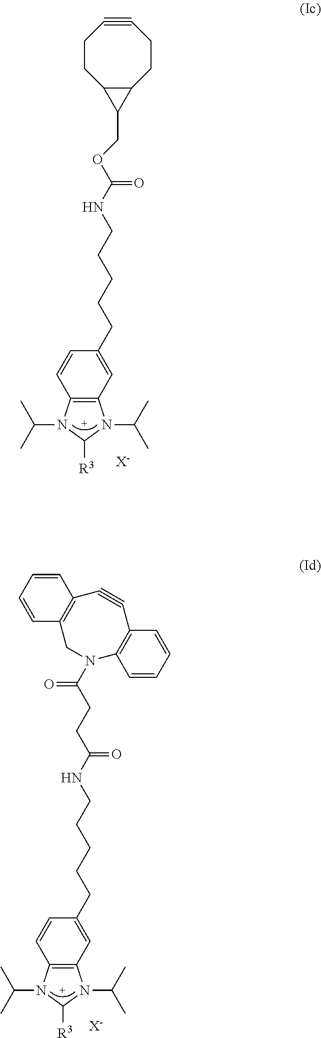

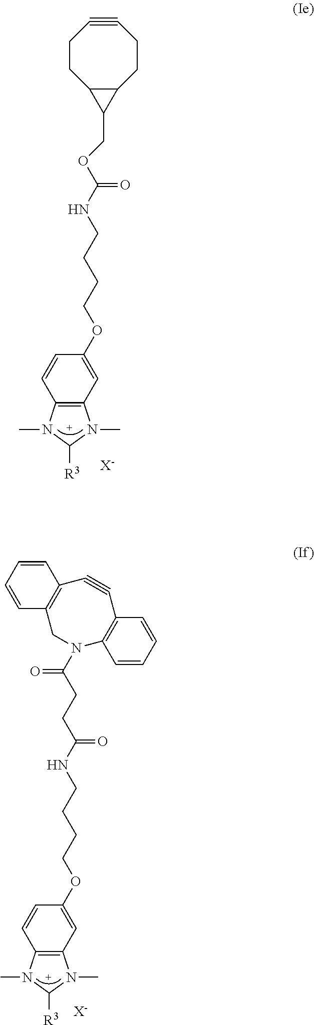

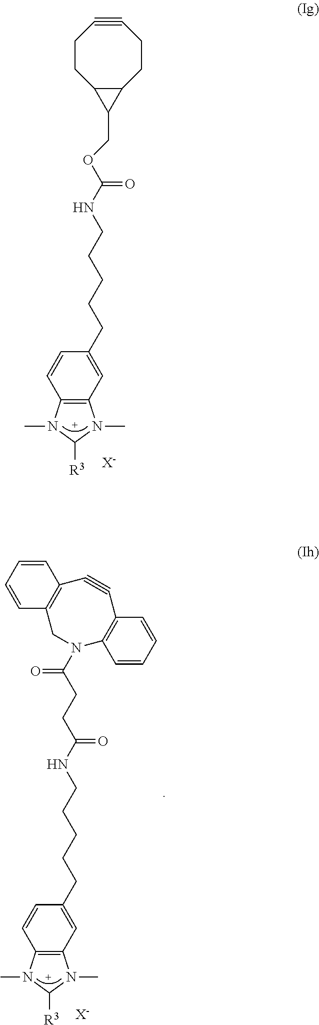

9. The compound of claim 1, wherein the compound is selected from compounds 5, 6, 7, 8, 9, and 10:

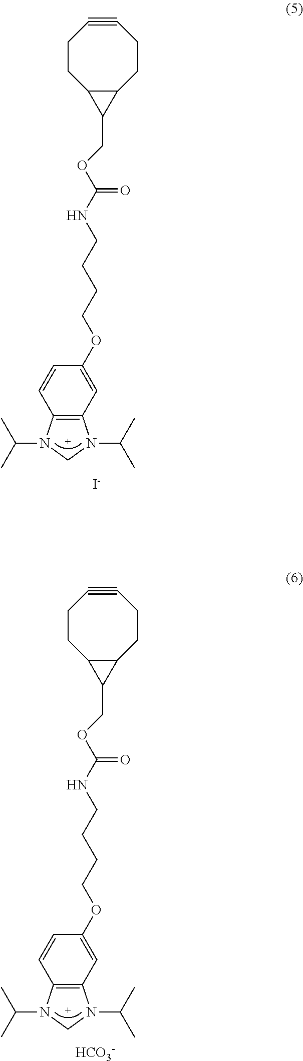

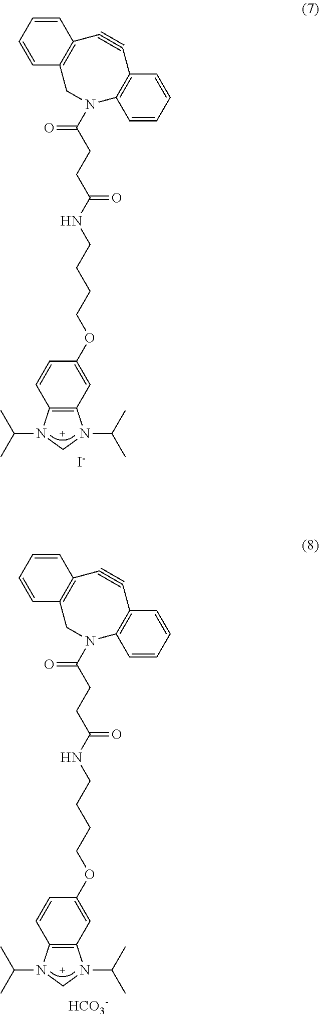

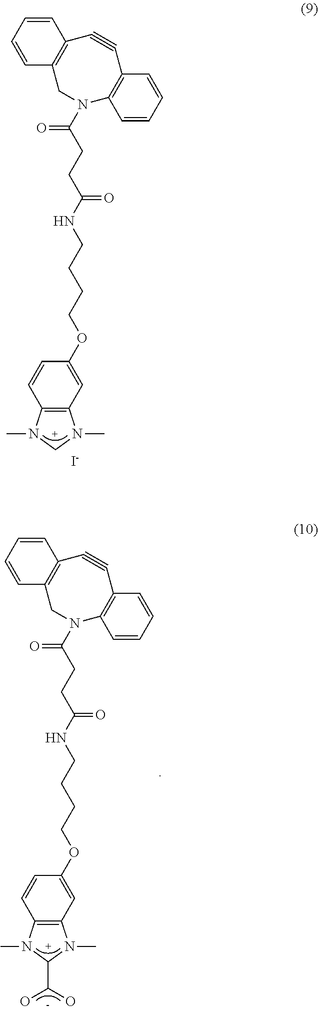

10. A method of preparing an N-heterocyclic carbene compound of structural formula I 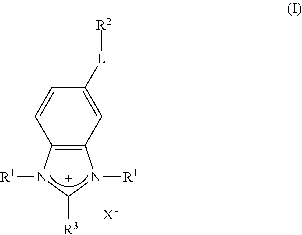
wherein,
R¹ is selected from —CH₃, and —CH₂—(CH₃)₂;
R² is a cyclooctyne group selected from IIa and IIb: 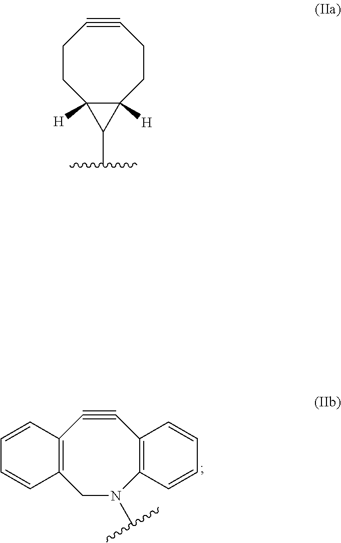 [SMILES: *C1[C@H]2CCC#CCC[C@@H]12 ; *N1Cc2ccccc2C#Cc2ccccc21]
L comprises a linker of structural formula L_{A} or L_{B} 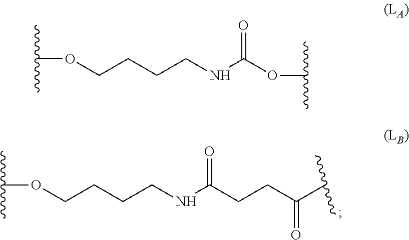 [SMILES: *OCCCCNC(=O)O* ; *OCCCCNC(=O)CCC(*)=O]
R³ is selected from —H and —COO⁻; and
X⁻ is an optional anion present when R³ is —H wherein,
the method comprising:
(a) preparing a Boc-protected precursor compound 3 or 4 according to the synthesis steps of Scheme 1: 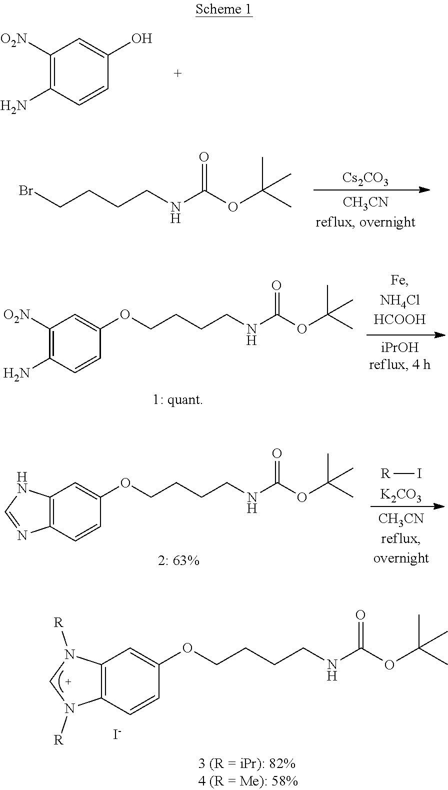
(b) reacting the precursor compound 3 of step (a) with BCN—NHS to prepare a BCN-functionalized NHC iodide salt of compound 5 or bicarbonate salt of compound 6, or with DBCO—NHS to prepare a DBCO-functionalized NHC iodide salt of compound 7 or bicarbonate salt of compound 8 according to the synthesis steps of Scheme 2A: 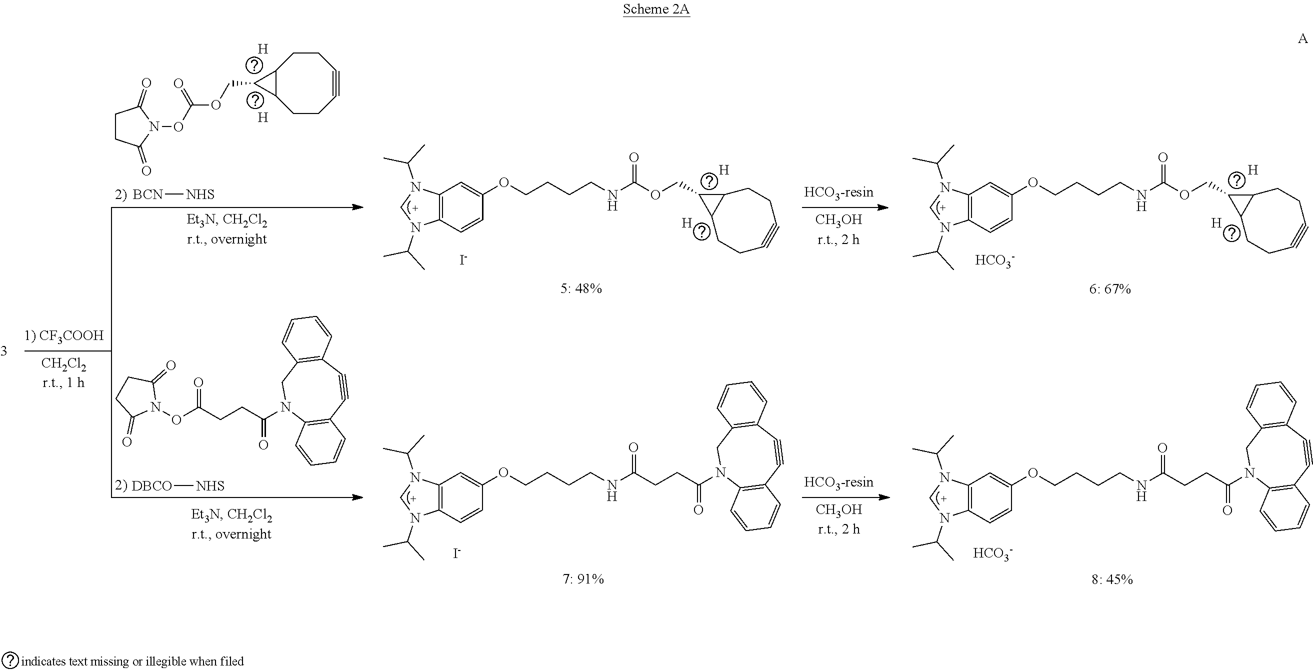
or
reacting the precursor compound 4 of step (a) with DBCO—NHS to prepare a DBCO-functionalized NHC iodide salt of compound 9 or a carboxylate adduct of compound 10 according to the synthesis steps of Scheme 2B:

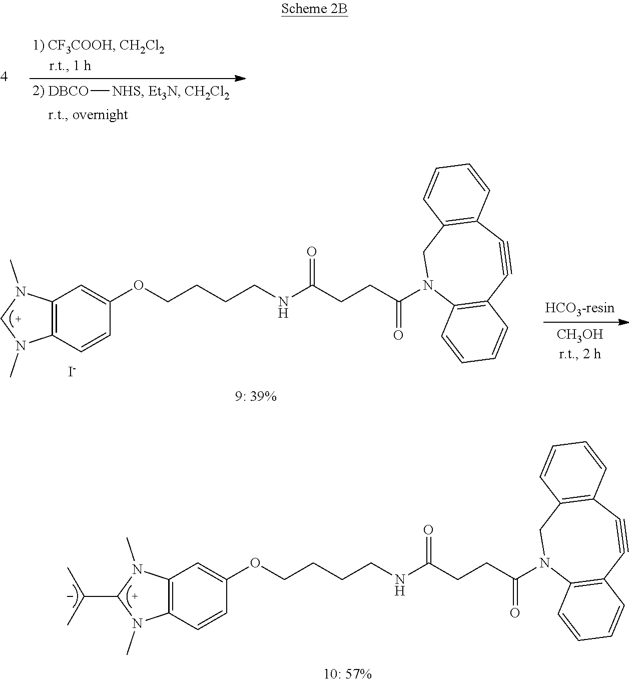

11. A composition comprising an N-heterocyclic carbene metal adduct of structural formula III 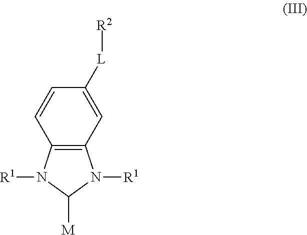
wherein,
R¹ is selected from —H, —CH₃, —CH₂—(CH₃)₂, —(C₁-C₆) linear or branched alkyl;
R² is a cyclooctyne group;
L is a linker comprising a covalently bonded chain of 2 to 100 atoms; and
M is an atom of a metal surface.

12. The composition of claim 12, wherein R¹ is selected from —CH₃, and —CH₂—(CH₃)₂.

13. The composition of claim 12, wherein the linker L comprises one or more of the following chemical groups: linear (C₁-C₅) alkyl, linear (C₁-C₅) alkenyl, linear (C₁-C₅) alkynyl, ester, ether, amine, amide, imide, phosphodiester, and/or polyethylene glycol (PEG).

14. The composition of claim 12, wherein L comprises a linker of structural formula L_{A}, L_{B}, or L_{C}

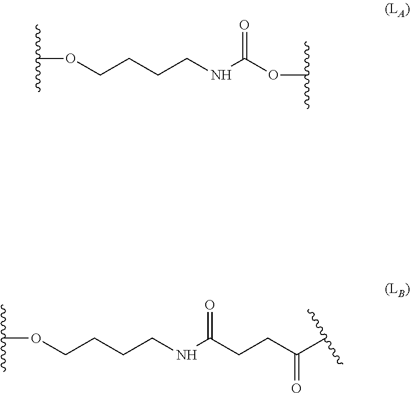 [SMILES: *OCCCCNC(=O)O* ; *OCCCCNC(=O)CCC(*)=O]

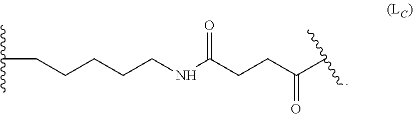 [SMILES: *CCCCCNC(=O)CCC(*)=O]

15. The composition of claim 12, wherein R² is a cyclooctyne group selected from IIa and IIb:

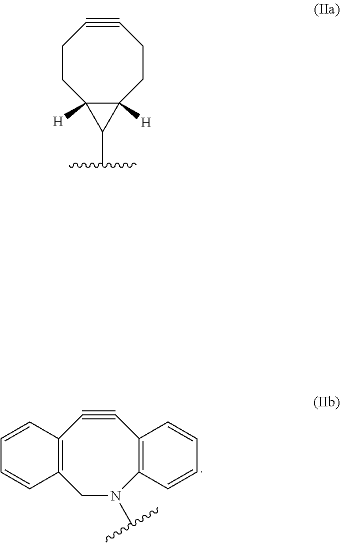 [SMILES: *C1[C@H]2CCC#CCC[C@@H]12 ; *N1Cc2ccccc2C#Cc2ccccc21]

16. The composition of claim 12, wherein the compound of structural formula III is selected from structural formulae IIIa, IIIb, IIIc, IIId, IIIe, IIIf, IIIg, and IIIh:

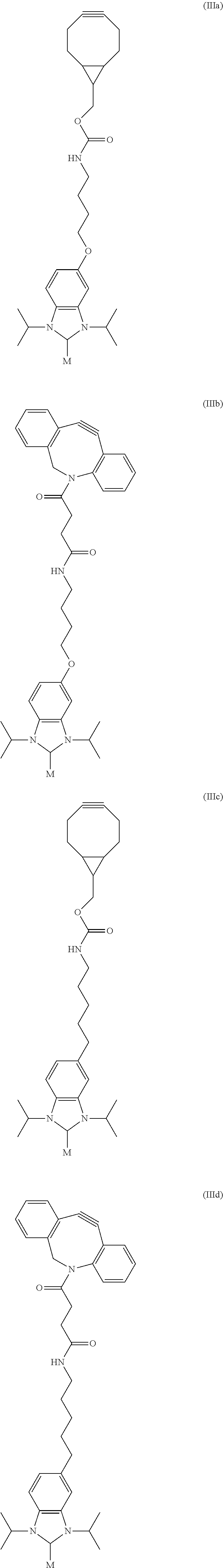

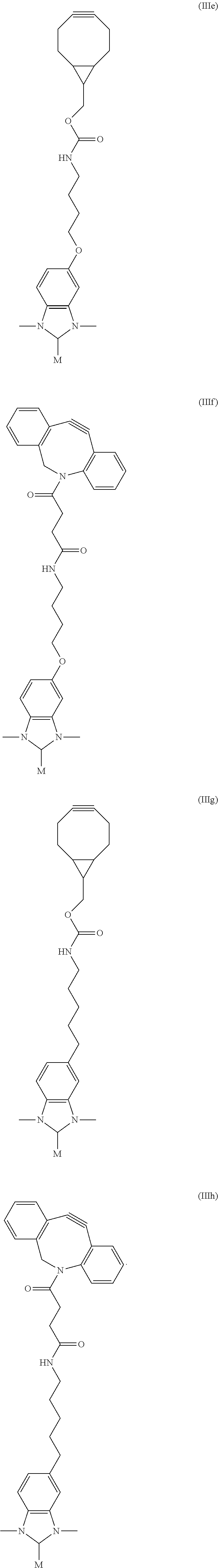

17. The composition of claim 12, wherein the atom M of the metal surface is selected from Au, Ag, Pd, Pt, Zn, Cd, and Se; optionally, wherein the metal surface is on a nanoparticle or on an electrode.

18. The composition of claim 12, wherein the metal surface further comprises a blocking N-heterocyclic carbene metal adduct of structural formula VI 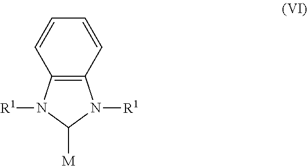
wherein,
R¹ is selected from —H, —CH₃, —CH₂—(CH₃)₂, —(C₁-C₆) linear or branched alkyl; and
M is an atom of the metal surface.

19. The composition of claim 22, wherein the blocking N-heterocyclic carbene metal adduct of structural formula VI is selected from compounds VIa and VIb

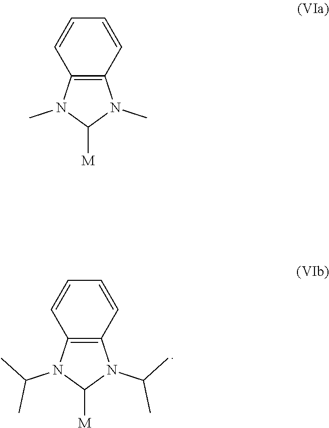

20. The composition of claim 22, wherein the mole ratio on the surface of the N-heterocyclic carbene metal adduct of structural formula III to the blocking N-heterocyclic carbene metal adduct of structural formula VI is at least about 1:100, at least about 1:250, at least about 1:500, at least about 1:1250, at least about 1:2500, or at least about 1:5000; optionally, wherein the mole ratio on the surface of the N-heterocyclic carbene metal adduct of structural formula III to the blocking N-heterocyclic carbene metal adduct of structural formula VI is between about 1:100 and 1:5000, between about 1:250 and 1:2500, or between about 1:500 and 1:1500.

21. A method for preparing composition comprising an N-heterocyclic carbene metal adduct of structural formula III 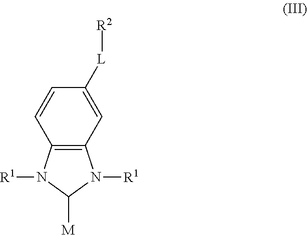
wherein,
R¹ is selected from —H, —CH₃, —CH₂—(CH₃)₂, —(C₁-C₆) linear or branched alkyl;
R² is a cyclooctyne group;
L is a linker comprising a covalently bonded chain of 2 to 100 atoms;
M is an atom of a metal surface;
the method comprising
(a) preparing a N-heterocyclic carbene compound of structural formula I according to method of claim 11; and
(b) depositing the compound on the metal surface under vacuum deposition conditions.

22. The method of claim 21, wherein the atom M of the metal surface is selected from Au, Ag, Pd, Pt, Zn, Cd, and Se; optionally, wherein the metal surface is on a nanoparticle or on an electrode.

23. A composition comprising an N-heterocyclic carbene metal adduct of structural formula IV 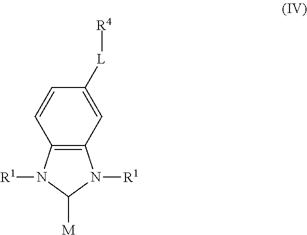
wherein,
R¹ is selected from —H, —CH₃, —CH₂—(CH₃)₂, —(C₁-C₆) linear or branched alkyl;
L is a linker comprising a covalently bonded chain of 2 to 100 atoms;
M is an atom of a metal surface; and
R⁴ is a cyclooctyl-triazole adduct comprising structural formula Va or Vb 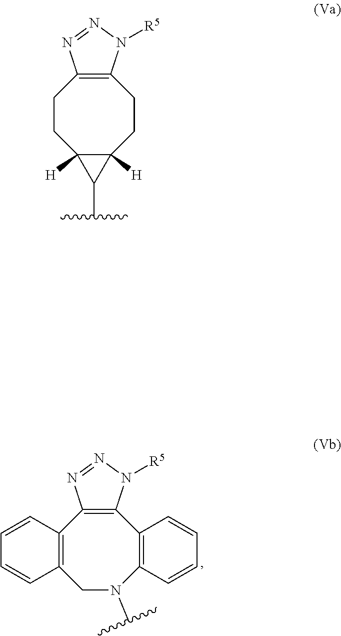
wherein, R⁵ comprises a linker moiety covalently attached to a redox-reporter modified aptamer.

24. The composition of claim 23, wherein the aptamer comprises an oligonucleotide;
optionally, wherein the oligonucleotide is: (i) attached to the linker through its 5′-end and attached to the redox-reporter through its 3′-end; or (ii) is attached to the linker through its 3′-end and attached to the redox-reporter through its 5′-end.

25. The composition of claim 23, wherein the redox-reporter comprises a compound selected from methylene blue, methylene blue, thionine, anthraquinone, anthraquinone-C5, Nile blue, neutral red, gallocyanine, dabcyl, 2,6-dichlorophenal-indophenol, ROX, ferrocene, pentamethyl ferrocene, ferrocene-C5, viologen, and Atto MB2.

26. The composition of claim 30, wherein the compound of structural formula IV is selected from IVa, IVb, IVc, IVd, IVe, IVf, IVg, and IVh:

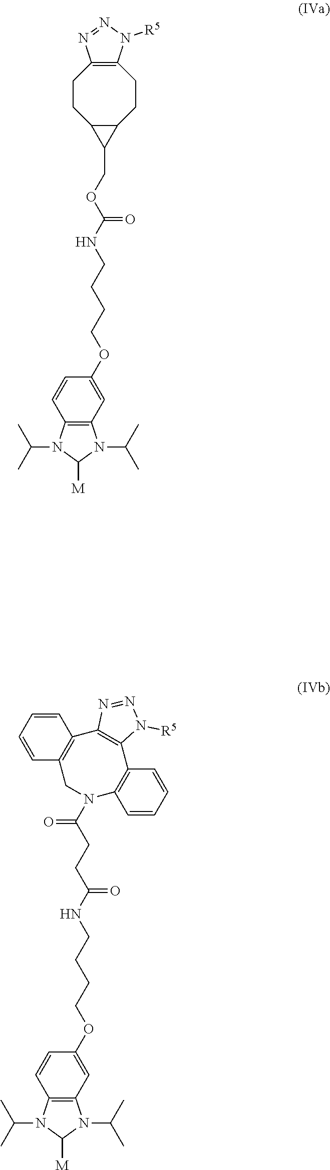

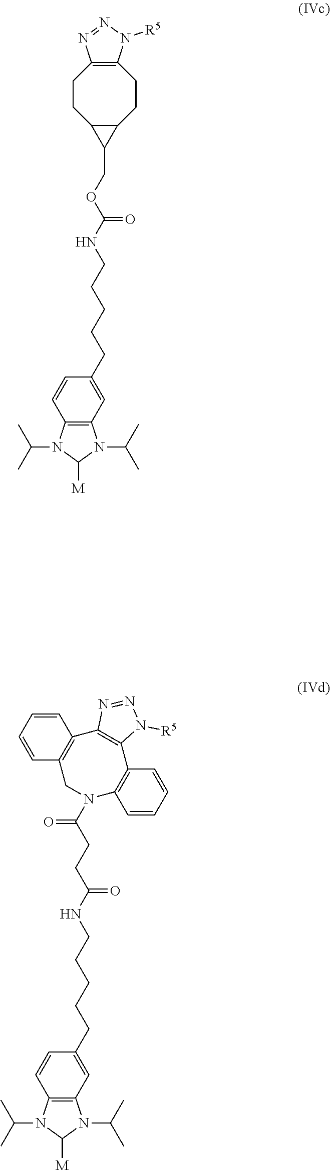

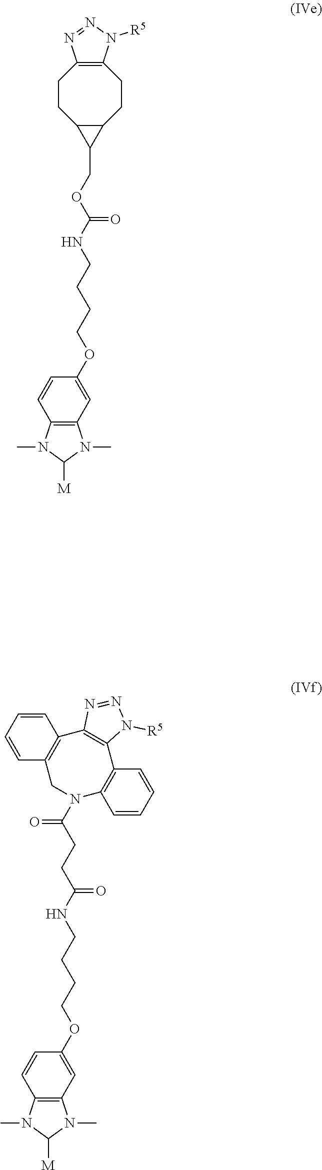

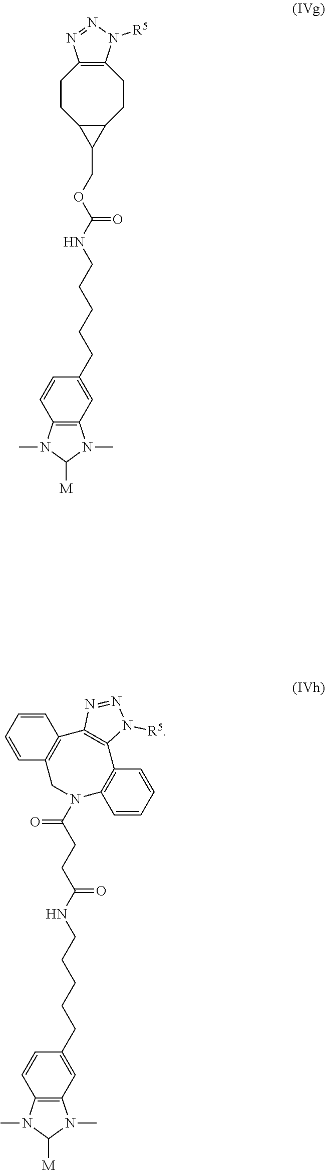

27. The composition of claim 23, wherein M is Au, the metal surface is on an electrode, and the redox-reporter modified aptamer is an oligonucleotide modified at its 3′-end with methylene blue.
